(19) United States
(12) Patent Application Publication
(10) Pub. No.: US 2023/0212275 A1
(43) Pub. Date: Jul. 6, 2023

(54) NUCLEIC ACID ENCODING AN ANTI-VEGF ENTITY AND A NEGATIVE COMPLEMENT REGULATOR AND USES THEREOF FOR THE TREATMENT OF AGE-RELATED MACULAR DEGENERATION

(71) Applicant: Gyroscope Therapeutics Limited, London (GB)

(72) Inventors: Josephine Joel, Watton at Stone (GB); Julian Esteve-Rudd, London (GB); Lawrence Tam, Richmond (GB); Scott Ellis, Reading (GB)

(73) Assignee: Gyroscope Therapeutics Limited, London (GB)

(21) Appl. No.: 17/928,803

(22) PCT Filed: Oct. 18, 2021
(86) PCT No.: PCT/GB2021/052689

§ 371 Date: Nov. 30, 2022

(30) Foreign Application Priority Data
Oct. 16, 2020 (GB) 2016463.8
Mar. 24, 2021 (GB) 2104148.8

(51) Int. Cl. C07K 16/22; C07K 14/71; C07K 14/47; C12N 15/113; A61P 27/02; C12N 15/86

(52) U.S. Cl. CPC C07K 16/22; C07K 14/71; C07K 14/4703; C12N 15/1136; A61P 27/02; C12N 15/86; C07K 2319/00; A61K 38/00

(57) ABSTRACT

The present invention relates to a product comprising (i) an anti-VEGF entity; and (ii) a negative complement regulator, or nucleotide sequences encoding therefor, as a combined preparation for simultaneous, separate or sequential use in therapy. In particular, the anti-VEGF entity is an anti-VEGF antibody, preferably aflibercept and the negative complement regulator is Complement Factor I (CFI) or Complement Factor H Like Protein 1 (FHL1). The main uses are for the treatment of eye diseases, in particular age-related macular degeneration (AMD).

### FIELD OF THE INVENTION

The present invention relates to agents for use in gene therapy. In particular, the invention relates to combinations of an anti-VEGF entity and a negative complement regulator, or nucleotide sequences encoding therefor, and their uses in the treatment or prevention of complement-mediated and complement-associated disorders, including complement-mediated eye diseases, such as age-related macular degeneration (AMD).

### BACKGROUND TO THE INVENTION

The macula is a small area in the retina of the eye, approximately 3 to 5 millimetres in size, adjacent to the optic nerve. It is the most sensitive area of the retina and contains the fovea, a depressed region that allows for high visual acuity and contains a dense concentration of cones, the photoreceptors that are responsible for colour vision.

Age-related macular degeneration (AMD) is the most common cause of functional blindness in developed countries for persons over 50 years of age (Seddon, J. M., Epidemiology of age-related macular degeneration. In: Ogden, T. E., et al., eds. Ryan S. J., ed-in-chief. Retina Vol II. 3rd ed. St. Louis, Mo.: Mosby; 2001: 1039-1050). The wet form of AMD is associated with neovascularisation originating from the choroidal vasculature and extending into the subretinal space. In addition, AMD is characterised by progressive degeneration of the retina, retinal pigment epithelium (RPE), and underlying choroid (the highly vascular tissue that lies beneath the RPE, between the retina and the sclera).

A variety of factors including oxidative stress, inflammation with a possible autoimmune component, genetic background (e.g. mutations), and environmental or behavioural factors such as smoking and diet may contribute to the pathogenesis of AMD.

The clinical progression of AMD is characterised in stages according to changes in the macula. The hallmark of early AMD is the appearance of drusen, which are accumulations of extracellular debris underneath the retina and appear as yellow spots in the retina during clinical examination and on fundus photographs. Drusen are categorised by size as small (<63 μm), medium (63-124 μm) and large (>124 μm). They are also considered as hard or soft depending on the appearance of their margins on ophthalmological examination. While hard drusen have clearly defined margins, soft drusen have less defined, fluid margins. The Age-related Eye Disease Study (AREDS) fundus photographic severity scale is one of the main classification systems used for this condition.

AMD has been classified into “dry” and “wet” (exudative or neovascular) forms. Dry AMD is typically characterised by progressive apoptosis of cells in the RPE layer, overlying photoreceptor cells, and frequently also the underlying cells in the choroidal capillary layer. Confluent areas of RPE cell death accompanied by overlying photoreceptor atrophy are referred to as geographic atrophy, which represent the late advanced stages of dry AMD. Patients with this form of AMD experience a slow and progressive deterioration in central vision.

Wet AMD is characterised by bleeding and/or leakage of fluid from abnormal vessels that have grown from the choroidal vessels (choriocapillaris) beneath the RPE and into the subretinal space in the macula, which can be responsible for sudden and disabling loss of vision. It has been estimated that much of the vision loss that patients experience is due to such choroidal neovascularisation (CNV) and its secondary complications. A subtype of neovascular AMD is termed retinal angiomatous proliferation (RAP). Here, angiomatous proliferation originates from the retina and extends posteriorly into the subretinal space, eventually communicating in some cases with choroidal new vessels. Polypoidal choroidal vasculopathy (PCV) is also known to occur.

Current treatment options for wet AMD are principally a number of therapies which target the Vascular Endothelial Growth Factor (VEGF) pathway. Examples of such VEGF-targeted therapies include the aptamer pegaptanib (N Engl J Med (2004) 351: 2805-2816) and antibodies such as ranibizumab (N Engl J Med (2006) 355: 1432-1444)), aflibercept (BMJ (2014) 98: i17-i21) and bevacizumab (BMJ (2010) 340: c2459). However, not all patients respond to treatment with an anti-VEGF antibody and either do not recover vision or progress to registered blindness. An incremental decline of visual acuity (VA) gains achieved with monthly treatment. has also been reported (Singer et al.; Ophthalmology; 2012; 119(6); 1175-83).

Further, there is evidence to support an association between anti-VEGF therapy and progression of geographic atrophy (Gemenetza et al.; Eye; 2017; 31; 1-9, Enslow et al.; Ophthalmology and Eye Diseases; 2016; 8; 21-32 & Sadda et al.; Ophthalmology; 2020; 127(5); 648-659).

There is also a need for a therapeutic strategy to reduce drug burden to patients and address problems with adherence and/or underdosing.

Accordingly, there is a significant need in the art for new approaches to treat eye diseases, such as AMD.

### SUMMARY OF THE INVENTION

Without wishing to be bound by theory, the present invention is based—at least in part—on the insight that it may be advantageous to target underlying mechanisms typically associated with “dry” AMD at the same time as targeting mechanisms typically associated with “wet” AMD. In addition, the invention is based in part on consideration of how the therapeutic targeting of one form of AMD may impact the other.

In a first aspect, the present invention provides a product comprising (i) an anti-VEGF entity; and (ii) a negative complement regulator, or nucleotide sequences encoding therefor, as a combined preparation for simultaneous, separate or sequential use in therapy.

In one embodiment, the product may be one or more polynucleotides which between them encode (i) an anti-VEGF entity; and (ii) a negative complement regulator.

Thus in a preferred embodiment, there is provided a polynucleotide that encodes an anti-VEGF entity and a negative complement regulator.

Preferably, the polynucleotide is a bicistronic polynucleotide encoding an anti-VEGF entity and a negative complement regulator.

In particular, the applicant has provided vectors that can be used for the delivery of both an anti-VEGF entity and a negative complement regulator to a patient. In particular, the applicant has successfully designed functional AAV vectors that can be produced at good titres and comprise nucleotide sequences encoding both an anti-VEGF entity and a negative complement regulator.

The anti-VEGF entity may be selected from an Ig fusion protein, an antibody, an aptamer, a polypeptide, a peptide, a polynucleotide (e.g. an antisense oligonucleotide, siRNA, shRNA, CRISPRi guidestrand of a sequence that targets the VEGF gene sequence or that of its mRNA) and a non-antibody scaffold. Suitably, the anti-VEGF entity is selected from aflibercept, ranibizumab, bevacizumab, brolucizumab and pegaptanib or a fragment or variant thereof.

The negative complement regulator may be selected from Complement Factor I (CFI), Complement Factor H Like Protein 1 (FHL1), Complement Factor H (CFH), Complement receptor type 1 (CR1), Membrane Cofactor Protein (MCP), Complement decay-accelerating factor (DAF) and MAC-inhibitory protein (MAC-IP), C1-inhibitor, anaphylatoxins inhibitor, C4b binding protein (C4BP), clusterin, vitronectin or a fragment or variant thereof.

In some embodiments, the polynucleotide further comprises a nucleotide sequence encoding a CMV promoter or a chicken p-actin promoter. Preferably, the CMV or CAG promoter is upstream of the nucleotide sequences encoding the anti-VEGF entity and the negative complement regulator. The chicken p-actin promoter may be provided in combination with a CMV early enhancer (i.e. as a CAG promoter).

In some embodiments, the polynucleotide further comprises a nucleotide sequence encoding a WPRE regulatory element. Preferably, the WPRE regulatory element is downstream of the nucleotide sequences encoding the anti-VEGF entity and the negative complement regulator.

In some embodiments, the polynucleotide further comprises a nucleotide sequence encoding a poly-A signal. Preferably, wherein the poly-A signal is downstream of the nucleotide sequences encoding the anti-VEGF entity and the negative complement regulator.

In some embodiments, the polynucleotide further comprises a nucleotide sequence encoding a Bovine Growth Hormone poly-A signal. Preferably, wherein the Bovine Growth Hormone poly-A signal is downstream of the nucleotide sequences encoding the anti-VEGF entity and the negative complement regulator.

In some embodiments, the polynucleotide further comprises nucleotide sequences encoding:
(a) a CMV promoter or CAG promoter, wherein the CMV promoter or CAG promoter is upstream of the nucleotide sequences encoding the anti-VEGF entity and the negative complement regulator; and
(b) a Bovine Growth Hormone poly-A signal, wherein the Bovine Growth Hormone poly-A signal is downstream of the nucleotide sequences encoding the anti-VEGF entity and the negative complement regulator.

In other embodiments, the polynucleotide further comprises nucleotide sequences encoding:
(a) a CMV promoter or CAG promoter, wherein the CMV promoter or CAG promoter is upstream of the nucleotide sequences encoding the anti-VEGF entity and the negative complement regulator;
(b) a WPRE regulatory element, wherein the WPRE regulatory element is downstream of the nucleotide sequences encoding the anti-VEGF entity and the negative complement regulator; and
(c) a Bovine Growth Hormone poly-A signal, wherein the Bovine Growth Hormone poly-A signal is downstream of the nucleotide sequences encoding the anti-VEGF entity and the negative complement regulator.

The WPRE regulatory element may be a WPRE3 regulatory element.

The nucleotide sequence encoding the anti-VEGF entity may be upstream of the nucleotide sequence encoding the negative complement regulator.

In some embodiments, the nucleotide sequence encoding the anti-VEGF entity is upstream of the nucleotide sequence encoding the negative complement regulator, wherein the anti-VEGF entity is selected from aflibercept, ranibizumab, bevacizumab, brolucizumab and pegaptanib or a fragment or variant thereof. In some embodiments, the nucleotide sequence encoding the anti-VEGF entity is upstream of the nucleotide sequence encoding the negative complement regulator, wherein the anti-VEGF entity is aflibercept or a fragment or variant thereof. In some embodiments, the nucleotide sequence encoding the anti-VEGF entity is upstream of the nucleotide sequence encoding the negative complement regulator, wherein the negative complement regulator is CFI or a variant or fragment thereof. In some embodiments, the nucleotide sequence encoding the anti-VEGF entity is upstream of the nucleotide sequence encoding the negative complement regulator, wherein the negative complement regulator is FHL1 or a variant or fragment thereof.

In some embodiments, the nucleotide sequence encoding the anti-VEGF entity is upstream of the nucleotide sequence encoding the negative complement regulator, wherein the anti-VEGF entity is aflibercept or a fragment or variant thereof and the negative complement regulator is CFI or a variant or fragment thereof.

In some embodiments, the nucleotide sequence encoding the anti-VEGF entity is upstream of the nucleotide sequence encoding the negative complement regulator, wherein the anti-VEGF entity is aflibercept or a fragment or variant thereof and the negative complement regulator is FHL1 or a variant or fragment thereof.

The nucleotide sequence encoding the negative complement regulator may be upstream of the nucleotide sequence encoding the anti-VEGF entity.

In some embodiments, the nucleotide sequence encoding the negative complement regulator is upstream of the nucleotide sequence encoding the anti-VEGF entity, wherein the anti-VEGF entity is selected from aflibercept, ranibizumab, bevacizumab, brolucizumab and pegaptanib or a fragment or variant thereof. In some embodiments, the nucleotide sequence encoding the negative complement regulator is upstream of the nucleotide sequence encoding the anti-VEGF entity, wherein the anti-VEGF entity is aflibercept or a fragment or variant thereof. In some embodiments, the nucleotide sequence encoding the negative complement regulator is upstream of the nucleotide sequence encoding the anti-VEGF entity, wherein the negative complement regulator is CFI or a variant or fragment thereof. In some embodiments, the nucleotide sequence encoding the negative complement regulator is upstream of the nucleotide sequence encoding the anti-VEGF entity, wherein the negative complement regulator is FHL1 or a variant or fragment thereof.

In some embodiments, the nucleotide sequence encoding the negative complement regulator is upstream of the nucleotide sequence encoding the anti-VEGF entity, wherein the anti-VEGF entity is aflibercept or a fragment or variant thereof and the negative complement regulator is CFI or a variant or fragment thereof.

In some embodiments, the nucleotide sequence encoding the negative complement regulator is upstream of the nucleotide sequence encoding the anti-VEGF entity, wherein the anti-VEGF entity is aflibercept or a fragment or variant thereof and the negative complement regulator is FHL1 or a variant or fragment thereof.

In some embodiments, the nucleotide sequences encoding the anti-VEGF entity and the negative complement regulator are operably linked by a linker. In some embodiments, the linker is a Furin, GSG, 11aa1 D and F2A linker. In preferred embodiments, the linker contains a self-cleaving 2A peptide sequence, for example P2A or a sequence which comprises or is defined by a Furin cleavage site, GSG, 11a1D and an F2A sequence.

In some embodiments, the polynucleotide further comprises one or more adeno-associated virus (AAV) inverted terminal repeats (ITRs). In preferred embodiments, the polynucleotide further comprises two AAV ITRs.

In some embodiments, the polynucleotide comprises an AAV ITR at its 5′ end and an AAV ITR at its 3′ end.

In some embodiments, the polynucleotide comprises:
(a) a 5′ AAV ITR;
(b) a CMV promoter or a CAG promoter;
(c) a nucleotide sequence encoding an anti-VEGF entity;
(d) a linker, optionally wherein the linker comprises a Furin cleavage site, GSG, 11a1D and an F2A sequence;
(e) a nucleotide sequence encoding a negative complement regulator;
(f) a poly-A signal, preferably a Bovine Growth Hormone poly-A signal; and
(g) a 3′ AAV ITR.

In some embodiments, the polynucleotide comprises:
(a) a 5′ AAV ITR;
(b) a CMV promoter or a CAG promoter;
(c) a nucleotide sequence encoding an anti-VEGF entity;
(d) a linker, optionally wherein the linker comprises a Furin cleavage site, GSG, 11a1D and an F2A sequence;
(e) a nucleotide sequence encoding a negative complement regulator;
(f) a WPRE regulatory element, optionally wherein the WPRE regulatory element is a WPRE3 regulatory element;
(g) a poly-A signal, preferably a Bovine Growth Hormone poly-A signal; and
(h) a 3′ AAV ITR.

Suitably the polynucleotides defined by (c) and (e) may be in the reciprocal position in the polynucleotide.

In particular, in some embodiments, the polynucleotide comprises:
(a) a 5′ AAV ITR;
(b) a CMV promoter or a CAG promoter;
(c) a nucleotide sequence encoding a negative complement regulator;
(d) a linker, optionally wherein the linker comprises a Furin cleavage site, GSG, 11a1D and an F2A sequence;
(e) a nucleotide sequence encoding an anti-VEGF entity;
(f) a poly-A signal, preferably a Bovine Growth Hormone poly-A signal; and
(g) a 3′ AAV ITR.

In some embodiments, the polynucleotide comprises:
(a) a 5′ AAV ITR;
(b) a CMV promoter or a CAG promoter;
(c) a nucleotide sequence encoding a negative complement regulator;
(d) a linker, optionally wherein the linker comprises a Furin cleavage site, GSG, 11a1D and an F2A sequence;
(e) a nucleotide sequence encoding an anti-VEGF entity;
(f) a WPRE regulatory element, optionally wherein the WPRE regulatory element is a WPRE3 regulatory element;
(g) a poly-A signal, preferably a Bovine Growth Hormone poly-A signal; and
(h) a 3′ AAV ITR.

Suitably, in embodiments where (b) comprises a CAG promoter; (f) may be a WPRE3 regulatory element.

The polynucleotide may comprise (a)-(g) or (a)-(h) in order, from 5′ to 3′.

In some embodiments, the AAV ITRs are AAV2 or AAV8 ITRs. In preferred embodiments, the AAV ITRs are AAV2 ITRs.

In some embodiments, the nucleotide sequences encoding the anti-VEGF entity and/or negative complement regulator are codon optimised. In some embodiments, the nucleotide sequence encoding the anti-VEGF entity is codon optimised. In some embodiments, the nucleotide sequence encoding the negative complement regulator is codon optimised. In preferred embodiments, the nucleotide sequences encoding aflibercept and CFI, FHL1 or CFH are codon optimised.

In some embodiments, the nucleotide sequence encoding aflibercept has at least 75%, 80%, 85%, 86%, 87%, 88%, 89%, 90%, 91%, 92%, 93%, 94%, 95%, 96%, 97%, 98% or 99% identity to SEQ ID NO:11.

In preferred embodiments, the nucleotide sequence encoding aflibercept is SEQ ID NO: 11.

In some embodiments, the nucleotide sequence encoding CFI has at least 75%, 80%, 85%, 86%, 87%, 88%, 89%, 90%, 91%, 92%, 93%, 94%, 95%, 96%, 97%, 98% or 99% identity to SEQ ID NO: 35 or 36.

In preferred embodiments, the nucleotide sequence encoding CFI is SEQ ID NO: 35 or 36.

In some embodiments, the nucleotide sequence encoding aflibercept is SEQ ID NO: 11 and the nucleotide sequence encoding CFI is SEQ ID NO: 35 or 36.

In some embodiments, the nucleotide sequence encoding FHL1 has at least 75%, 80%, 85%, 86%, 87%, 88%, 89%, 90%, 91%, 92%, 93%, 94%, 95%, 96%, 97%, 98% or 99% identity to SEQ ID NO: 41.

In preferred embodiments, the nucleotide sequence encoding FHL1 is SEQ ID NO: 41.

In some embodiments, the nucleotide sequence encoding aflibercept is SEQ ID NO: 11 and the nucleotide sequence encoding FHL1 is SEQ ID NO: 41.

In some embodiments, the polynucleotide comprises the nucleotide sequence of SEQ ID NO: 45, or a nucleotide sequence that has at least 75%, 80%, 85%, 86%, 87%, 88%, 89%, 90%, 91%, 92%, 93%, 94%, 95%, 96%, 97%, 98% or 99% sequence identity thereto.

In some embodiments, the polynucleotide comprises the nucleotide sequence of SEQ ID NO: 46, or a nucleotide sequence that has at least 75%, 80%, 85%, 86%, 87%, 88%, 89%, 90%, 91%, 92%, 93%, 94%, 95%, 96%, 97%, 98% or 99% sequence identity thereto.

In some embodiments, the polynucleotide comprises the nucleotide sequence of SEQ ID NO: 47, or a nucleotide sequence that has at least 75%, 80%, 85%, 86%, 87%, 88%, 89%, 90%, 91%, 92%, 93%, 94%, 95%, 96%, 97%, 98% or 99% sequence identity thereto.

In some embodiments, the polynucleotide comprises the nucleotide sequence of SEQ ID NO: 48, or a nucleotide sequence that has at least 75%, 80%, 85%, 86%, 87%, 88%, 89%, 90%, 91%, 92%, 93%, 94%, 95%, 96%, 97%, 98% or 99% sequence identity thereto.

In some embodiments, the polynucleotide comprises the nucleotide sequence of SEQ ID NO: 49, or a nucleotide sequence that has at least 75%, 80%, 85%, 86%, 87%, 88%, 89%, 90%, 91%, 92%, 93%, 94%, 95%, 96%, 97%, 98% or 99% sequence identity thereto.

In some embodiments, the polynucleotide comprises the nucleotide sequence of SEQ ID NO: 50, or a nucleotide sequence that has at least 75%, 80%, 85%, 86%, 87%, 88%, 89%, 90%, 91%, 92%, 93%, 94%, 95%, 96%, 97%, 98% or 99% sequence identity thereto.

In some embodiments, the polynucleotide comprises the nucleotide sequence of SEQ ID NO: 51, or a nucleotide sequence that has at least 75%, 80%, 85%, 86%, 87%, 88%, 89%, 90%, 91%, 92%, 93%, 94%, 95%, 96%, 97%, 98% or 99% sequence identity thereto.

In some embodiments, the polynucleotide comprises the nucleotide sequence of SEQ ID NO: 52, or a nucleotide sequence that has at least 75%, 80%, 85%, 86%, 87%, 88%, 89%, 90%, 91%, 92%, 93%, 94%, 95%, 96%, 97%, 98% or 99% sequence identity thereto.

In some embodiments, the polynucleotide comprises the nucleotide sequence of SEQ ID NO: 53, or a nucleotide sequence that has at least 75%, 80%, 85%, 86%, 87%, 88%, 89%, 90%, 91%, 92%, 93%, 94%, 95%, 96%, 97%, 98% or 99% sequence identity thereto.

In some embodiments, the polynucleotide comprises the nucleotide sequence of SEQ ID NO: 54, or a nucleotide sequence that has at least 75%, 80%, 85%, 86%, 87%, 88%, 89%, 90%, 91%, 92%, 93%, 94%, 95%, 96%, 97%, 98% or 99% sequence identity thereto.

In some embodiments, the polynucleotide is less than or equal to 5.2, 5.1, 5.0, 4.9, 4.8 or 4.7 kb. In preferred embodiments, the polynucleotide is less than or equal to 4.7 kb.

In another aspect, the invention provides a vector comprising the polynucleotide of the invention. Thus according to one aspect the invention provides a vector comprising (i) polynucleotide encoding an anti-VEGF entity and (ii) a polynucleotide encoding a negative complement regulator. Preferably the anti-VEGF entity and the negative complement regulator are provided on the same polynucleotide, which is preferably a bicistronic polynucleotide.

Suitably, a bicistronic polynucleotide as used herein may refer to a polynucleotide comprising at least two protein coding regions each operably linked to the same promoter region. Suitably, the bicistronic polypeptide may comprise two protein coding regions each operably linked to the same promoter region.

Suitably, the present invention also provides more than one (e.g. two) vector which between them comprise (i) polynucleotide encoding an anti-VEGF entity and (ii) a polynucleotide encoding a negative complement regulator.

In some embodiments, the vector is an adeno-associated viral (AAV), retroviral, lentiviral or adenoviral vector.

In preferred embodiments, the vector is an AAV vector.

In some embodiments, the vector is in the form of a viral vector particle.

In some embodiments, the AAV vector particle comprises an AAV2 or AAV8 genome, preferably an AAV2 genome.

In some embodiments, the AAV vector particle comprises AAV2 or AAV8 capsid proteins.

In some embodiments, the AAV vector particle comprises an AAV2 genome and AAV2 capsid proteins (AAV2/2). In preferred embodiments, the AAV vector particle comprises an AAV2 genome and AAV8 capsid proteins (AAV2/8).

In another aspect, the invention provides a cell comprising the polynucleotide of the invention.

In another aspect, the invention provides a cell transduced with the vector of the invention.

In another aspect, the invention provides a pharmaceutical composition comprising the polynucleotide, vector or cell of the invention in combination with a pharmaceutically acceptable carrier, diluent or excipient.

In preferred embodiments, the pharmaceutical composition is for intraocular administration.

In another aspect, the invention provides the product, polynucleotide, vector, cell or pharmaceutical composition of the invention for use in therapy.

In another aspect, the invention provides the product, polynucleotide, vector, cell or pharmaceutical composition of the invention for use in treating or preventing an ocular disorder.

In another aspect, the invention provides the product, polynucleotide, vector, cell or pharmaceutical composition of the invention for use in treating or preventing a complement-mediated disorder of the eye.

In another aspect, the invention provides a method of treating or preventing a complement-mediated disorder of the eye comprising administering the product, polynucleotide, vector, cell or pharmaceutical composition of the invention to a subject in need thereof.

In another aspect, the invention provides a method of providing an anti-VEGF entity and a negative complement regulator to a subject, comprising delivering the product, polynucleotide, vector or cell of the invention to the eye of the subject.

In particular embodiments, the product, polynucleotide, vector, cell or pharmaceutical composition of the invention is used in the treatment of complement-mediated disorders, particularly chronic inflammatory conditions and even more particularly, those which are associated with overactivity of the complement C3b feedback cycle.

In some embodiments, the disorder is associated with over-activity of the complement C3b feedback cycle and/or under-activity of the C3b breakdown cycle (see FIG. 5).

In some embodiments, the disorder is age-related macular degeneration (AMD) or diabetic retinopathy. In other embodiments, the disorder is glaucoma, Stargardt's disease, central serous chorioretinopathy, retinitis pigmentosa, polypoidal choroidal vasculopathy, diabetic macular edema, branch retinal vein occlusion or uveitis.

In preferred embodiments, the disorder is AMD.

In some embodiments, the disorder is wet AMD and/or dry AMD. In some embodiments, the product, polynucleotide, vector, cell or pharmaceutical composition is used to treat and/or prevent wet AMD and dry AMD simultaneously.

In some embodiments, the AMD is wet AMD and the use according to the invention further prevents and/or treats onset of dry AMD in said subject. In some embodiments, the AMD is dry AMD and the use according to the invention further prevents and/or treats onset of wet AMD in said subject.

In some embodiments, the subject has been diagnosed with AMD or is at risk of acquiring AMD.

In some embodiments, the use is for treating or preventing a disorder in a subject:
(a) having lower than normal Complement Factor I activity or concentration in the eye and/or serum, preferably having a concentration of, or activity equivalent to, 0-30, 0-20 or 0-10 μg/mL in serum; and/or
(b) being heterozygous or homozygous for an age-related macular degeneration (AMD)-associated SNP, preferably a rare Complement Factor I and/or Complement Factor H variant.

In some embodiments, the use is for treating or preventing a disorder in a subject:
(a) having a normal level of Complement Factor I activity or concentration in the eye and/or serum, preferably at least 30 μg/mL, such as 30-40 μg/mL in serum; and/or
(b) not carrying a rare Complement Factor I variant allele.

In another aspect, the invention provides the product, polynucleotide, vector, cell or pharmaceutical composition of the invention for use in treating or preventing diabetic retinopathy.

In some embodiments, the formation of geographic atrophy is prevented or reduced, and/or the amount of geographic atrophy is reduced.

In some embodiments, the progression of geographic atrophy is slowed.

In some embodiments, there is at least a 10% reduction in the increase in geographic atrophy area over the 12 months following administration to a treated eye of a subject, relative to an untreated eye over the same period. In other embodiments, there is at least 20%, 30%, 40%, 50%, 60%, 70%, 80% or 90% reduction in the increase in geographic atrophy area over the 12 months following administration to a treated eye of a subject, relative to an untreated eye over the same period

In some embodiments, administration of the product, polynucleotide, vector, cell or pharmaceutical composition increases the level of C3b-inactivating and iC3b-degradation activity in a subject, or in an eye, such as in the retinal pigment epithelium (RPE) aqueous humor and/or vitreous humor, of a subject, optionally to a level that exceeds a normal level in a subject, or eye or RPE thereof.

In another aspect, the invention provides the product, polynucleotide, vector, cell or pharmaceutical composition of the invention for use in improving or restoring vision or visual acuity, for example in a subject suffering from an eye disorder, such as an eye disorder disclosed herein. In another aspect, the invention provides the product, polynucleotide, vector or cell of the invention for use in mitigating loss of vision or visual acuity, for example a loss of vision or visual acuity associated with an eye disorder, such as an eye disorder disclosed herein.

In another aspect, the invention provides the product, polynucleotide, vector, cell or pharmaceutical composition of the invention for use in improving or restoring reading speed in a subject, for example in a subject suffering from an eye disorder, such as an eye disorder disclosed herein. In another aspect, the invention provides the product, polynucleotide, vector or cell of the invention for use in mitigating reduction in reading speed in a subject, for example a reduction in reading speed associated with an eye disorder, such as an eye disorder disclosed herein.

In another aspect, the invention provides the product, polynucleotide, vector, cell or pharmaceutical composition of the invention for use in reducing or preventing loss of photoreceptors and/or the retinal pigment epithelium (RPE), for example a loss of photoreceptors and/or the RPE associated with an eye disorder, such as an eye disorder disclosed herein.

In a further aspect, the invention provides the product, polynucleotide, vector, cell or pharmaceutical composition of the invention for use in reducing or preventing neovascularization, vascular leakage or retina edema with an eye disorder, such as an eye disorder disclosed herein.

In some embodiments, the product, polynucleotide, vector, cell or pharmaceutical composition is administered intraocularly.

In some embodiments, the product, polynucleotide, vector, cell or pharmaceutical composition is administered to the eye of a subject by subretinal, direct retinal, suprachoroidal or intravitreal injection.

In some embodiments, the product, polynucleotide, vector, cell or pharmaceutical composition is administered to the eye of a subject by subretinal injection.

In some embodiments, the product, polynucleotide, vector, cell or pharmaceutical composition of the invention is not administered systemically. In other embodiments, the product, polynucleotide, vector, cell or pharmaceutical composition of the invention is not administered intravenously.

### DESCRIPTION OF THE DRAWINGS

FIG. 1—Schematics depicting the design of illustrative monocistronic aflibercept vectors and AAV genome size (ITR-ITR)

FIG. 2—Comparison of aflibercept expression between different codon optimised AAV2-vectors. Data show two independent experiments

FIG. 3—Representative images of Western blots performed under (A) non-reducing and (B) reducing conditions illustrating aflibercept protein expression in cultured supernatant 72 hours post-transduction. (C) Densitometric analysis of aflibercept expression under non-reducing and reducing conditions from two independent experiments

FIG. 4—Schematic diagrams illustrating the design of illustrative bicistronic vectors expressing negative complement regulator and aflibercept

FIG. 5—C3b feedback (amplification) and breakdown (down-regulation) cycles of the alternative pathway of vertebrate complement (“I”=Complement Factor I; “H”=Complement Factor H; “B”=Complement Factor B; and “D”=Complement Factor D).

FIG. 6—Administration of monocistronic vectors expressing CFI (GT005) or FHL1 (RC001) in a mouse model of CNV

FIG. 7—Comparison of bicistronic vector expression in HEK293 cells. Representative expression levels of (A) AAV8 bicistronic expression of Aflibercept and CFI; (B) AAV2 bicistronic expression of Aflibercept (left hand bar) and CFI (right hand bar) at an MOI of 5 e3; (C) AAV8 bicistronic expression of Aflibercept and FHL-1 and (D) AAV2 bicistronic expression of Aflibercept (left hand bar) and FHL-1 (right hand bar) at an MOI of 5 e3; (E) AAV8 bicistronic expression of Aflibercept and CFI; (F) AAV8 bicistronic expression of Aflibercept and FHL-1.

FIG. 8—Western blot analysis showing protein expression from bicistronic vectors under non-reducing conditions.

FIG. 9—Binding affinity of Aflibercept. Affinities of Aflibercept expressed by monocistronic and bicistronic vectors for VEGFA, as determined by using a binding assay that measures unbound VEGFA after incubation of a fixed concentration of human VEGFA with varying concentrations of vector derived Aflibercept. IC50 value of Eylea® derived from Kd curve=2.3 nM

FIG. 10—Inhibitory properties of Aflibercept. Inhibition of VEGFA-induced proliferation of HUVEC cells using varying concentration of vector derived Aflibercept in the presence of a fixed concentration of VEGFA.

FIG. 11—C3b cleavage assays for CFI and FHL1 demonstrated that CFI and FHL1 expressed from bicistronic vectors is functional and can facilitate breakdown of C3b to iC3b.

FIG. 12—Aflibercept mRNA expression—monocistronic aflibercept (mouse). AAV8 (monocistronic) vectors expressing anti-VEGF (Aflibercept) were administered subretinally into the right eye of nine-week-old male mice. Transgene-derived mRNA was quantified by reverse transcription and qRT-PCR.

FIG. 13—Aflibercept protein expression—monocistronic aflibercept (mouse). AAV8 (monocistronic) vectors expressing anti-VEGF (Aflibercept) were administered subretinally into the right eye of nine-week-old male mice. Unbound aflibercept in ocular fluids was measured using a quantitative sandwich type ELISA.

FIG. 14—Aflibercept mRNA expression—bicistronic candidates (mouse). Bicistronic AAV vectors co-expressing antiVEGF (Aflibercept) and complement regulators (hCFI or FHL-1) were administered subretinally into the right eye (OD) of 10-week-old male mice. Transgene-derived mRNA was quantified by reverse transcription and qRT-PCR.

FIG. 15—Aflibercept, CFI and FHL-1 protein expression (bicistronic vectors). Bicistronic AAV vectors co-expressing antiVEGF (Aflibercept) and complement regulators (hCFI or FHL-1) were administered subretinally into the right eye (OD) of 10-week-old male mice. Unbound aflibercept in ocular fluids was measured using a quantitative sandwich type ELISA. Human CFI and FHL-1 in ocular fluids was quantified using MSD's electrochemiluminescence detection technology.

FIG. 16—In vivo efficacy in murine laser-induced choroidal neovascularisation model. Five groups of mice received unilateral (right eye) subretinal injections of different AAV vectors. Four weeks after administration of the vectors, CNV was induced by laser photocoagulation. Mice were imaged at days four and seven post lasering, and then sacrificed. Quantitation of (A) CNV leakage area and (B) CNV lesion size was carried out.

### DETAILED DESCRIPTION OF THE INVENTION

The terms “comprising”, “comprises” and “comprised of” as used herein are synonymous with “including” or “includes”; or “containing” or “contains”, and are inclusive or open-ended and do not exclude additional, non-recited members, elements or steps. The terms “comprising”, “comprises” and “comprised of” also include the term “consisting of”.

VEGF

Vascular endothelial growth factor (VEGF) is a potent angiogenic protein produced by cells that stimulates the formation of blood vessels. VEGF is a sub-family of growth factors, the platelet-derived growth factor family of cystine-knot growth factors. They are involved in both vasculogenesis (the de novo formation of the embryonic circulatory system) and angiogenesis (the growth of blood vessels from pre-existing vasculature).

In mammals, the VEGF family comprises five members: VEGF-A, placenta growth factor (PGF), VEGF-B, VEGF-C and VEGF-D.

Suitably, the VEGF referred to herein may be VEGF-A.

There are multiple isoforms of VEGF-A that result from alternative splicing of mRNA from a single, 8-exon VEGFA gene. These are classified into two groups which are referred to according to their terminal exon (exon 8) splice site: the proximal splice site (denoted VEGFₓₓₓ) or distal splice site (VEGFₓₓₓb). In addition, alternate splicing of exon 6 and 7 alters their heparin-binding affinity and amino acid number (in humans: VEGF₁₂₁, VEGF₁₂₁b, VEGF₁₄₅, VEGF₁₆₅, VEGF₁₆₅b, VEGF₁₈₉, VEGF₂₀₆; the rodent orthologs of these proteins contain one fewer amino acids). These domains have important functional consequences for the VEGF splice variants, as the terminal (exon 8) splice site determines whether the proteins are pro-angiogenic (proximal splice site, expressed during angiogenesis, VEGFₓₓₓ) or anti-angiogenic (distal splice site, expressed in normal tissues, VEGFₓₓₓb). In addition, inclusion or exclusion of exons 6 and 7 mediate interactions with heparin sulfate proteoglycans (HSPGs) and neurophilin co-receptors on the cell surface, enhancing their ability to bind and activate the VEGF receptors (VEGFRs).

VEGF interacts with a family of 3 receptor tyrosine kinases: VEGFR1 (VEGF receptor 1), VEGFR2, and VEGFR3. VEGFA and VEGFB bind to VEGFR1, VEGFA binds to VEGFR2, and VEGFC and VEGFD bind to both VEGFR2 and VEGFR3. Placental growth factor (PGF) primarily interacts with VEGFR1. The VEGFRs are found on a wide variety of cell types. VEGFR1, also called Flt-1 (fms-like tyrosine kinase 1), is found on vascular endothelial cells, hematopoietic stem cells, monocytes, and macrophages. VEGFR2, also called KDR (kinase insert domain) or Flk-1 (fetal liver kinase 1), is expressed on vascular and lymphatic endothelial cells; VEGFR3 (also called Flt-4) is restricted to lymphatic endothelial cells. On ligand binding, VEGFRs transduce intracellular signals through a variety of mediators. In the case of VEGFR2, which is the best characterized, these include phosphotidylinositol-3 kinase (PI3K)/Akt, mitogen-activated kinases, the nonreceptor tyrosine kinase Src, as well as PLCγ (phospholipase C gamma)/PKC (protein kinase C), which promote angiogenesis, lymphangiogenesis, vascular permeability, and vascular homeostasis.

VEGF-A is important in the aetiology of diabetic retinopathy (DR). The microcirculatory problems in the retina of people with diabetes can cause retinal ischaemia, which results in the release of VEGF-A, and a switch in the balance of pro-angiogenic VEGF isoforms over the normally expressed anti-angiogenic VEGF isoforms. Pro-angiogenic VEGF may then cause the creation of new blood vessels in the retina and elsewhere in the eye, heralding changes that may impact vision.

VEGF-A is also important in the disease pathology of the wet form age-related macular degeneration (AMD), which is the leading cause of blindness for the elderly of the industrialized world. The vascular pathology of AMD shares certain similarities with diabetic retinopathy, although the cause of disease and the typical source of neovascularization differs between the two diseases.

As used herein, an anti-VEGF entity may refer to any entity which is capable of reducing the expression and/or activity of VEGF.

As will be apparent, the anti-VEGF entity will be capable of reducing the expression and/or activity of one or more pro-angiogenic isoforms of VEGF, in particular VEGF-A.

Suitably, the VEGF may be VEGFA, VEGFB and/or PGF.

Suitably, the VEGF may be VEGFA, VEGFB and PGF.

The anti-VEGF entity may be selected from one or more of the following: an antibody, an Ig fusion protein, a polypeptide, a peptide, a polynucleotide (e.g. an antisense oligonucleotide, siRNA, shRNA), a small molecule, a non-antibody scaffold, an aptamer, or combinations thereof.

Antibody as used herein is intended to encompass both full antibodies and antibody fragments thereof. Suitably antibodies include, but are not limited to, monoclonal and polyclonal antibodies, engineered antibodies including chimeric, CDR-grafted and humanised antibodies, single-chain antibodies, antibody fragments and artificially selected antibodies produced using phage display or alternative techniques.

Suitable antibody fragments capable of binding to a selected target, include Fv, ScFv, F(ab′) and F(ab′)2. In addition, alternatives to classical antibodies may also be used in the invention, for example “avibodies”, “avimers”, “anticalins”, “nanobodies” and “DARPins”.

Reference to “scFv” or “single-chain variable fragment” as used herein includes molecules wherein the variable heavy (VH) and variable light chain (VL) of an antibody are linked via a flexible oligopeptide. A scFv is thus a fusion between at least one variable heavy and at least one variable light chain.

As will be apparent, in embodiments where the anti-VEGF entity and negative complement regulator are provided as an isolated polynucleotide, the anti-VEGF entity will be an entity that can be encoded by a polynucleotide. For example, the anti-VEGF entity may be an Ig fusion protein, an antibody or antigen binding fragment thereof, a polypeptide, a peptide, a polynucleotide (e.g. an antisense oligonucleotide, siRNA, shRNA, CRISPRi guidestrand of a sequence that targets the VEGF gene sequence or that of its mRNA), a non-antibody scaffold, or an aptamer.

The anti-VEGF entity may act by binding to one or more VEGFs and/or one or more VEGF receptors.

The anti-VEGF entity may be capable of reducing a pro-proliferative and/or pro-angiogenic activity of VEGF.

By way of example, the anti-VEGF entity may be capable of reducing the proliferation of a VEGF dependent cell line. Suitable cell lines include, but are not limited to endothelial cells such as Human umbilical vein endothelial cells (HUVEC) and the Ba/F3-VEGFR2 cell line described by Wentink et al. (Br J Cancer; 2016; 115(8); 940-948).

The anti-VEGF entity may reduce the proliferation of a VEGF dependent cell line by at least 1-fold (suitably, at least 1.5-fold, at least 2-fold, at least 2.5-fold, at least 3-fold, at least 4-fold or at least 5-fold) relative to the proliferation in the absence of anti-VEGF entity. The anti-VEGF entity may reduce the proliferation of a VEGF dependent cell line by at least 10% (suitably, at least 20%, at least 30%, at least 40%, at least 50%, at least 100%, at least 150% or at least 200%) relative to the proliferation in the absence of the anti-VEGF entity.

The anti-VEGF entity may be selected from aflibercept, ranibizumab, bevacizumab and pegaptanib or a fragment or variant thereof.

The anti-VEGF entity may be selected from aflibercept, ranibizumab and pegaptanib or a fragment or variant thereof.

The anti-VEGF entity may be selected from aflibercept and ranibizumab or a fragment or variant thereof.

The anti-VEGF entity may be aflibercept or a fragment or variant thereof.

The anti-VEGF entity may be ranibizumab or a fragment or variant thereof.

The anti-VEGF entity may be bevacizumab or a fragment or variant thereof.

The anti-VEGF entity maybe pegaptanib or a fragment or variant thereof.

The anti-VEGF entity may be aflibercept. Aflibercept binds to circulating VEGFs and acts as a “VEGF trap”. It thereby inhibits the activity of the VEGF subtypes VEGF-A and VEGF-B, as well as PGF. Aflibercept is a recombinant protein composed of the binding domains of VEGFR1 and VE GFR2 fused with the Fc region of human immunoglobulin gamma 1 (IgG1). Structurally, aflibercept is a dimeric glycoprotein with a protein molecular weight of 96.9 kDa.

An illustrative aflibercept amino acid sequence is shown as SEQ ID NO: 1.

SEQ ID NO: 1
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDFNWEYPSSKHQHKKL
VNRDLKTQSGSEMKKFLSTLTIDGVTRSDQGLYTCAASSGLMTKKNSTFV
RVHEKDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPG

The aflibercept amino acid sequence may comprise a sequence shown as SEQ ID NO: 1. The variant may have at least 75%, 80%, 85%, 86%, 87%, 88%, 89%, 90%, 91%, 92%, 93%, 94%, 95%, 96%, 97%, 98% or 99% identity to SEQ ID NO: 1. Preferably, a fragment or variant substantially retains a functional activity of the protein shown as SEQ ID NO: 1.

The aflibercept amino acid sequence may be encoded by a polynucleotide sequence shown as SEQ ID NO: 2-11.

SEQ ID NO: 2
AGTGATACAGGTAGACCTTTCGTAGAGATGTACAGTGAAATCCCCGAAAT
TATACACATGACTGAAGGAAGGGAGCTCGTCATTCCCTGCCGGGTTACGT
CACCTAACATCACTGTTACTTTAAAAAAGTTTCCACTTGACACTTTGATC
CCTGATGGAAAACGCATAATCTGGGACAGTAGAAAGGGCTTCATCATATC
AAATGCAACGTACAAAGAAATAGGGCTTCTGACCTGTGAAGCAACAGTCA
ATGGGCATTTGTATAAGACAAACTATCTCACACATCGACAAACCAATACA
ATCATAGATGTCGTTCTGAGTCCGTCTCATGGAATTGAACTATCTGTTGG
AGAAAAGCTTGTCTTAAATTGTACAGCAAGAACTGAACTAAATGTGGGGA
TTGACTTCAACTGGGAATACCCTTCTTCGAAGCATCAGCATAAGAAACTT
GTAAACCGAGACCTAAAAACCCAGTCTGGGAGTGAGATGAAGAAATTTTT
GAGCACCTTAACTATAGATGGTGTAACCCGGAGTGACCAAGGATTGTACA
CCTGTGCAGCATCCAGTGGGCTGATGACCAAGAAGAACAGCACATTTGTC
AGGGTCCATGAAAAAGACAAAACTCACACATGCCCACCGTGCCCAGCACC
TGAACTCCTGGGGGGACCGTCAGTCTTCCTCTTCCCCCCAAAACCCAAGG
ACACCCTCATGATCTCCCGGACCCCTGAGGTCACATGCGTGGTGGTGGAC
GTGAGCCACGAAGACCCTGAGGTCAAGTTCAACTGGTACGTGGACGGCGT
GGAGGTGCATAATGCCAAGACAAAGCCGCGGGAGGAGCAGTACAACAGCA
CGTACCGTGTGGTCAGCGTCCTCACCGTCCTGCACCAGGACTGGCTGAAT
GGCAAGGAGTACAAGTGCAAGGTCTCCAACAAAGCCCTCCCAGCCCCCAT
CGAGAAAACCATCTCCAAAGCCAAAGGGCAGCCCCGAGAACCACAGGTGT
ACACCCTGCCCCCATCCCGGGATGAGCTGACCAAGAACCAGGTCAGCCTG
ACCTGCCTGGTCAAAGGCTTCTATCCCAGCGACATCGCCGTGGAGTGGGA
GAGCAATGGGCAGCCGGAGAACAACTACAAGACCACGCCTCCCGTGCTGG
ACTCCGACGGCTCCTTCTTCCTCTACAGCAAGCTCACCGTGGACAAGAGC
AGGTGGCAGCAGGGGAACGTCTTCTCATGCTCCGTGATGCATGAGGCTCT
GCACAACCACTACACGCAGAAGAGCCTCTCCCTGTCTCCGGGTTGA

In some embodiments, the nucleotide sequences encoding aflibercept (or any other anti-VEGF entity) used in the invention are codon-optimised. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available.

Illustrative codon optimized nucleotide sequences encoding aflibercept are shown as SEQ ID NO: 3-11.

SEQ ID NO: 3
TCTGATACCGGCAGACCCTTCGTGGAAATGTACAGCGAGATCCCCGAGATCATCCACATGACCGAGGGCAGAGAG
CTGGTCATCCCCTGCAGAGTGACAAGCCCCAACATCACCGTGACTCTGAAGAAGTTCCCTCTGGACACACTGATC
CCCGACGGCAAGAGAATCATCTGGGACAGCCGGAAGGGCTTCATCATCAGCAACGCCACCTACAAAGAGATCGGC
CTGCTGACCTGTGAAGCCACCGTGAATGGCCACCTGTACAAGACCAACTACCTGACACACAGACAGACCAACACC
ATCATCGACGTGGTGCTGAGCCCTAGCCACGGCATTGAACTGTCTGTGGGCGAGAAGCTGGTGCTGAACTGTACC
GCCAGAACCGAGCTGAACGTGGGCATCGACTTCAACTGGGAGTACCCCAGCAGCAAGCACCAGCACAAGAAACTG
GTCAACCGGGACCTGAAAACCCAGAGCGGCAGCGAGATGAAGAAATTCCTGAGCACCCTGACCATCGACGGCGTG
ACCAGATCTGACCAGGGCCTGTACACATGTGCCGCCAGCTCTGGCCTGATGACCAAGAAAAACAGCACCTTCGTG
CGGGTGCACGAGAAGGACAAGACCCACACCTGTCCTCCATGTCCTGCTCCAGAACTGCTCGGCGGACCTTCCGTG
TTCCTGTTTCCTCCAAAGCCTAAGGACACCCTGATGATCAGCAGAACCCCTGAAGTGACCTGCGTGGTGGTGGAT
GTGTCCCACGAGGATCCCGAAGTGAAGTTCAATTGGTACGTGGACGGCGTGGAAGTGCACAACGCCAAGACCAAG
CCTAGAGAGGAACAGTACAATAGCACCTACAGAGTGGTGTCCGTGCTGACCGTGCTGCACCAGGATTGGCTGAAC
GGCAAAGAGTACAAGTGCAAGGTGTCCAACAAGGCCCTGCCTGCTCCTATCGAGAAAACCATCTCCAAGGCCAAG
GGCCAGCCTAGGGAACCCCAGGTTTACACACTGCCTCCAAGCAGGGACGAGCTGACAAAGAACCAGGTGTCCCTG
ACCTGCCTGGTCAAGGGCTTCTACCCTTCCGATATCGCCGTGGAATGGGAGAGCAATGGCCAGCCTGAGAACAAC
TACAAGACAACCCCTCCTGTGCTGGACAGCGACGGCTCATTCTTCCTGTACAGCAAGCTGACAGTGGACAAGAGC
AGATGGCAGCAGGGCAACGTGTTCAGCTGCAGCGTGATGCACGAGGCCCTGCACAACCACTACACCCAGAAGTCC
CTGAGCCTGTCTCCTGGCTAA
SEQ ID NO: 4
TCTGATACCGGCAGACCCTTCGTGGAAATGTACAGCGAGATCCCCGAGATCATTCACATGACCGAGGGCAGAGAG
CTGGTCATCCCCTGCCGAGTGACAAGCCCCAACATCACCGTGACTCTGAAGAAATTCCCTCTGGACACACTGATC
CCCGACGGCAAGAGAATCATTTGGGACAGCCGGAAGGGCTTCATCATTAGCAACGCCACCTACAAAGAGATCGGC
CTGCTCACCTGTGAAGCCACCGTGAATGGCCACCTGTACAAGACCAACTACCTGACACACAGACAGACCAACACC
ATCATTGACGTGGTCCTGAGCCCTAGCCACGGCATTGAACTGTCTGTGGGCGAGAAGCTGGTGCTGAACTGTACC
GCCAGAACCGAGCTGAACGTGGGCATCGACTTCAACTGGGAGTACCCCAGCTCCAAGCACCAGCACAAGAAACTG
GTCAACCGGGACCTGAAAACCCAGAGCGGCAGCGAGATGAAGAAATTCCTGAGCACCCTGACCATCGACGGCGTG
ACCAGATCTGACCAGGGCCTGTACACATGTGCCGCTAGCTCTGGCCTGATGACCAAGAAAAACAGCACCTTCGTG
CGGGTGCACGAGAAGGACAAGACCCACACCTGTCCTCCATGTCCTGCTCCCGAACTGCTCGGCGGACCTTCCGTG
TTCCTGTTTCCTCCAAAGCCTAAGGACACCCTGATGATCAGCAGAACCCCTGAAGTGACCTGCGTGGTCGTGGAT
GTGTCCCACGAGGATCCCGAAGTGAAGTTCAATTGGTACGTGGACGGCGTGGAAGTGCACAACGCCAAGACCAAG
CCTAGAGAGGAACAGTACAATAGCACCTACAGAGTGGTCTCCGTGCTGACCGTGCTGCACCAGGATTGGCTGAAC
GGCAAAGAGTACAAGTGCAAGGTGTCCAACAAGGCCCTGCCTGCTCCTATCGAGAAAACCATCTCCAAGGCCAAG
GGCCAGCCTAGGGAACCCCAGGTTTACACACTGCCTCCAAGCAGGGACGAGCTGACAAAGAACCAGGTGTCCCTG
ACCTGCCTGGTCAAGGGCTTCTACCCTTCCGATATCGCCGTGGAATGGGAGAGCAATGGCCAGCCTGAGAACAAT
TACAAGACAACCCCTCCCGTGCTGGACAGCGACGGCTCATTCTTTCTGTACTCCAAGCTGACAGTGGACAAGAGC
AGATGGCAGCAAGGCAACGTGTTCAGCTGCAGCGTGATGCACGAGGCCCTGCACAACCACTACACCCAGAAGTCC
CTGAGCCTGTCTCCTGGCTAA
SEQ ID NO: 5
AGCGACACCGGCAGACCCTTCGTGGAGATGTACTCCGAGATCCCTGAGATCATCCACATGACCGAGGGCAGGGAG
CTGGTCATCCCATGCCGCGTGACATCCCCCAACATCACCGTGACACTGAAGAAGTTCCCTCTGGACACCCTGATC
CCAGATGGCAAGCGGATCATCTGGGACTCTAGAAAGGGCTTTATCATCAGCAATGCCACATATAAGGAGATCGGC
CTGCTGACCTGCGAGGCCACAGTGAACGGCCACCTGTACAAGACCAATTATCTGACACACAGACAGACCAACACA
ATCATCGATGTGGTGCTGTCCCCATCTCACGGCATCGAGCTGAGCGTGGGCGAGAAGCTGGTGCTGAATTGTACC
GCCAGGACAGAGCTGAACGTGGGCATCGACTTCAATTGGGAGTACCCCAGCTCCAAGCACCAGCACAAGAAGCTG
GTGAACAGGGATCTGAAGACCCAGAGCGGCTCCGAGATGAAGAAGTTTCTGTCTACCCTGACAATCGACGGAGTG
ACCCGCAGCGATCAGGGACTGTATACATGCGCCGCCTCTAGCGGCCTGATGACCAAGAAGAACAGCACCTTCGTG
CGGGTGCACGAGAAGGACAAGACCCACACATGCCCACCTTGTCCAGCACCAGAGCTGCTGGGAGGACCAAGCGTG
TTCCTGTTTCCACCCAAGCCCAAGGATACCCTGATGATCTCTCGCACCCCCGAGGTGACATGCGTGGTGGTGGAC
GTGAGCCACGAGGACCCCGAGGTGAAGTTTAACTGGTACGTGGACGGCGTGGAGGTGCACAATGCCAAGACCAAG
CCTCGGGAGGAGCAGTACAACAGCACCTATAGAGTGGTGTCCGTGCTGACAGTGCTGCACCAGGATTGGCTGAAC
GGCAAGGAGTATAAGTGCAAGGTGTCCAATAAGGCCCTGCCTGCCCCAATCGAGAAGACCATCTCTAAGGCAAAG
GGACAGCCCAGGGAGCCTCAGGTGTACACACTGCCTCCATCCCGCGACGAGCTGACCAAGAACCAGGTGTCTCTG
ACATGTCTGGTGAAGGGCTTCTATCCTAGCGACATCGCCGTGGAGTGGGAGTCCAATGGCCAGCCAGAGAACAAT
TACAAGACCACACCCCCTGTGCTGGACTCCGATGGCTCTTTCTTTCTGTATTCCAAGCTGACCGTGGATAAGTCT
CGGTGGCAGCAGGGCAACGTGTTTTCTTGTAGCGTGATGCACGAGGCCCTGCACAATCACTACACACAGAAGTCC
CTGTCTCTGAGCCCCGGCTGA
SEQ ID NO: 6
AGCGACACCGGCAGACCCTTCGTGGAGATGTACTCCGAGATCCCTGAGATCATTCACATGACCGAGGGCAGGGAG
CTGGTCATCCCATGCCGCGTGACATCCCCCAACATCACCGTGACACTGAAGAAATTCCCTCTGGACACCCTGATC
CCAGATGGCAAGCGGATCATTTGGGACTCTAGAAAGGGCTTTATCATTAGCAATGCCACATATAAGGAGATCGGC
CTGCTCACCTGCGAGGCCACAGTGAACGGCCACCTGTACAAGACCAATTATCTGACACACAGACAGACCAACACA
ATCATTGATGTGGTCCTGTCCCCATCTCACGGCATCGAGCTGAGCGTGGGCGAGAAGCTGGTGCTGAATTGTACC
GCCAGGACAGAGCTGAACGTGGGCATCGACTTCAATTGGGAGTACCCCAGCTCCAAGCACCAGCACAAGAAACTG
GTGAACAGGGATCTGAAGACCCAGAGCGGCTCCGAGATGAAGAAATTTCTGTCTACCCTGACAATCGACGGAGTG
ACCCGCAGCGATCAGGGACTGTATACATGCGCCGCTTCTAGCGGCCTGATGACCAAGAAAAACAGCACCTTCGTG
CGGGTGCACGAGAAGGACAAGACCCACACATGCCCACCTTGTCCAGCACCAGAGCTGCTCGGAGGCCCAAGCGTG
TTCCTGTTTCCACCCAAGCCCAAGGATACCCTGATGATCTCTCGCACCCCCGAGGTGACATGCGTGGTCGTGGAC
GTGAGCCACGAGGACCCCGAGGTGAAGTTTAACTGGTATGTGGACGGCGTGGAGGTGCACAATGCCAAGACCAAG
CCTCGGGAGGAACAGTACAACAGCACCTATAGAGTGGTCTCCGTGCTGACAGTGCTGCACCAGGATTGGCTGAAC
GGCAAGGAGTATAAGTGCAAGGTGTCCAATAAGGCCCTGCCTGCCCCAATCGAGAAGACCATCTCTAAGGCAAAG
GGACAGCCCAGGGAGCCTCAGGTGTACACACTGCCTCCATCCCGCGACGAGCTGACCAAGAACCAGGTGTCTCTG
ACATGTCTGGTGAAGGGCTTCTATCCTAGCGACATCGCCGTGGAGTGGGAGTCCAATGGCCAGCCAGAGAACAAT
TACAAGACCACACCCCCTGTGCTGGACTCCGATGGCTCTTTCTTTCTGTATTCCAAGCTGACCGTGGATAAGTCT
CGGTGGCAGCAAGGCAACGTGTTTTCTTGTTCCGTGATGCACGAGGCCCTGCACAATCACTACACACAGAAGTCC
CTGTCTCTGAGCCCCGGCTGA
SEQ ID NO: 7
TCCGACACCGGCAGACCCTTCGTGGAGATGTACAGCGAGATCCCCGAGATCATTCACATGACCGAGGGCAGAGAG
CTCGTGATCCCTTGTAGAGTGACCTCCCCCAACATCACCGTGACACTGAAGAAGTTTCCTCTGGATACCCTCATC
CCCGACGGCAAGAGGATTATTTGGGACTCTAGAAAGGGCTTCATCATTAGCAACGCCACCTACAAGGAGATCGGA
CTGCTGACATGCGAGGCCACCGTGAACGGCCACCTCTACAAGACCAACTATCTGACCCATAGACAGACCAATACC
ATCATCGACGTGGTGCTCTCCCCTAGCCACGGAATCGAGCTGAGCGTGGGCGAGAAACTGGTGCTCAATTGCACC
GCTAGAACAGAGCTCAACGTGGGCATCGACTTCAACTGGGAATACCCCTCCTCCAAGCACCAACACAAGAAGCTC
GTGAATAGAGATCTGAAGACCCAAAGCGGCAGCGAGATGAAGAAGTTTCTGAGCACACTGACAATTGACGGCGTC
ACAAGAAGCGACCAAGGCCTCTATACATGCGCCGCCAGCAGCGGACTGATGACCAAGAAGAACAGCACCTTCGTG
AGAGTGCACGAGAAAGATAAGACCCACACATGTCCCCCTTGCCCCGCCCCCGAGCTGCTGGGAGGACCTAGCGTC
TTTCTGTTCCCTCCTAAGCCCAAGGACACACTGATGATCTCCAGAACCCCCGAGGTGACATGCGTGGTCGTGGAC
GTGTCCCACGAGGACCCCGAGGTGAAGTTCAATTGGTACGTCGACGGCGTGGAGGTGCACAATGCTAAGACCAAG
CCCAGAGAGGAGCAGTACAACTCCACCTACAGAGTGGTGTCCGTGCTGACCGTGCTCCATCAAGACTGGCTCAAC
GGCAAGGAGTACAAGTGCAAGGTGAGCAACAAGGCCCTCCCCGCCCCTATCGAGAAGACCATTTCCAAGGCCAAG
GGCCAGCCTAGAGAGCCCCAAGTGTATACACTGCCTCCCAGCAGAGACGAGCTCACCAAAAACCAAGTGTCCCTC
ACATGTCTGGTCAAAGGCTTCTACCCCAGCGATATCGCCGTGGAGTGGGAAAGCAACGGCCAGCCCGAGAACAAC
TACAAGACAACCCCTCCCGTGCTCGACAGCGACGGAAGCTTCTTTCTGTACAGCAAGCTGACCGTGGACAAGTCT
AGATGGCAGCAAGGCAATGTCTTTAGCTGCAGCGTGATGCACGAGGCTCTGCACAATCACTACACCCAGAAGAGC
CTCTCTCTGAGCCCCGGCTGA
SEQ ID NO: 8
TCCGACACCGGCAGACCCTTCGTGGAGATGTACAGCGAGATCCCCGAGATCATTCACATGACCGAGGGCAGAGAG
CTCGTGATCCCTTGTCGAGTGACCTCCCCCAACATCACCGTGACACTGAAGAAATTTCCTCTGGATACCCTCATC
CCCGACGGCAAGAGGATTATCTGGGACTCTAGAAAGGGCTTCATCATTAGCAACGCCACCTACAAGGAGATCGGA
CTGCTTACATGCGAGGCCACCGTGAACGGCCACCTCTACAAGACCAACTATCTGACCCATAGACAGACCAATACC
ATCATTGACGTGGTTCTCTCCCCTTCTCACGGAATCGAGCTGAGCGTGGGCGAGAAACTGGTGCTCAATTGCACC
GCTAGAACAGAGCTCAACGTGGGCATCGACTTCAACTGGGAATACCCCTCCTCTAAGCACCAACACAAGAAACTC
GTGAATAGAGATCTGAAGACCCAAAGCGGCAGCGAGATGAAGAAATTTCTGAGCACACTGACAATTGACGGCGTC
ACAAGAAGCGACCAAGGCCTCTATACATGCGCCGCTAGCAGTGGACTGATGACCAAGAAAAACAGCACCTTCGTG
AGAGTGCACGAGAAAGATAAGACCCACACATGTCCCCCTTGCCCCGCCCCCGAGCTGCTTGGAGGTCCTAGCGTC
TTTCTGTTCCCTCCAAAGCCCAAGGACACACTGATGATCTCCAGAACCCCCGAGGTGACATGCGTGGTCGTGGAC
GTGTCCCACGAGGACCCCGAGGTGAAGTTCAATTGGTATGTTGACGGCGTGGAGGTGCACAATGCTAAGACCAAG
CCCAGAGAGGAACAGTACAACTCCACCTACAGAGTGGTTTCCGTGCTGACCGTGCTCCATCAAGACTGGCTCAAC
GGCAAGGAGTACAAGTGCAAAGTGAGCAACAAGGCCCTCCCCGCCCCTATCGAGAAGACCATTTCCAAGGCCAAG
GGCCAGCCTAGAGAGCCCCAAGTGTATACACTGCCTCCCAGCAGAGACGAGCTCACCAAAAACCAAGTGTCCCTC
ACATGTCTGGTCAAAGGCTTCTACCCCAGCGATATCGCCGTGGAGTGGGAAAGCAACGGCCAGCCCGAGAACAAT
TACAAGACAACCCCTCCCGTGCTCGACAGCGACGGAAGCTTCTTTCTGTACTCCAAGCTGACCGTGGACAAGTCT
AGATGGCAGCAAGGCAATGTCTTTAGCTGCAGCGTGATGCACGAGGCTCTGCACAATCACTACACCCAGAAGAGC
CTCTCTCTGAGCCCCGGCTGA
SEQ ID NO: 9
TCCGACACCGGGAGACCGTTTGTAGAAATGTACTCTGAAATTCCAGAAATTATTCACATGACTGAAGGACGAGAA
CTCGTAATCCCGTGCCGGGTAACATCACCGAACATTACCGTTACTCTCAAGAAGTTCCCGTTGGACACTCTGATT
CCAGACGGCAAGCGGATTATATGGGATTCACGAAAAGGCTTTATCATCAGCAACGCTACATATAAGGAGATAGGC
CTCCTCACCTGCGAGGCCACAGTGAACGGACACCTTTACAAGACAAACTACCTCACGCATCGCCAGACTAACACT
ATTATAGACGTTGTACTCAGCCCTTCCCACGGAATAGAGCTCTCTGTTGGGGAAAAGCTTGTTCTGAACTGCACG
GCACGAACCGAACTCAACGTTGGTATTGATTTCAACTGGGAATACCCCTCTTCAAAGCACCAACATAAGAAGCTC
GTGAACAGGGACCTGAAGACGCAGAGTGGATCCGAAATGAAGAAATTTCTTTCAACTCTCACAATCGACGGCGTT
ACCAGGAGCGATCAGGGGCTTTATACTTGCGCGGCTTCTTCTGGACTCATGACAAAGAAAAACAGTACTTTCGTT
CGCGTCCATGAGAAAGATAAGACCCATACGTGCCCACCTTGTCCAGCTCCGGAACTTTTGGGCGGACCCAGCGTA
TTTCTTTTTCCTCCCAAGCCGAAAGACACACTGATGATATCAAGAACTCCTGAAGTTACATGTGTTGTTGTTGAC
GTTAGTCATGAAGACCCTGAGGTCAAGTTCAACTGGTATGTTGATGGAGTAGAGGTTCATAACGCGAAGACGAAG
CCACGAGAAGAACAGTACAACAGCACGTATAGGGTGGTCAGTGTACTTACGGTACTCCACCAAGATTGGCTTAAC
GGTAAGGAATACAAGTGTAAAGTATCTAATAAGGCATTGCCGGCTCCGATTGAAAAGACAATCTCTAAGGCAAAA
GGCCAGCCGCGCGAGCCGCAAGTATATACATTGCCTCCCTCAAGAGATGAGCTTACCAAGAACCAGGTGAGCCTG
ACATGCCTTGTCAAAGGATTCTACCCTAGCGACATAGCCGTCGAATGGGAATCAAACGGTCAACCAGAGAACAAT
TATAAAACCACCCCCCCGGTGCTTGATAGTGATGGCTCCTTTTTTCTGTATTCTAAGTTGACGGTGGATAAGAGT
CGGTGGCAACAGGGGAATGTATTTAGTTGCAGTGTGATGCACGAGGCTCTTCATAACCACTACACACAAAAATCT
TTGAGCTTGTCTCCAGGTTGA
SEQ ID NO: 10
TCCGACACCGGGAGACCGTTTGTAGAAATGTACTCTGAAATTCCAGAAATTATCCACATGACTGAAGGACGAGAA
CTCGTAATCCCGTGCCGGGTAACATCACCGAACATTACCGTTACTCTCAAGAAATTCCCGTTGGACACTCTGATT
CCAGACGGCAAGCGGATTATATGGGATTCACGAAAAGGCTTTATCATTAGCAACGCTACATATAAGGAGATAGGC
CTCCTGACCTGCGAGGCCACAGTGAACGGACACCTTTACAAGACAAACTACCTCACGCATCGCCAGACTAACACT
ATTATAGACGTTGTACTCAGCCCTTCCCACGGAATAGAGCTCTCTGTTGGGGAAAAGCTTGTTCTGAACTGCACG
GCACGAACCGAACTCAACGTTGGTATTGATTTCAACTGGGAATACCCCTCTTCAAAGCACCAACATAAGAAACTC
GTGAACAGGGACCTGAAGACGCAGAGTGGATCCGAAATGAAGAAATTTCTTTCAACTCTCACAATCGACGGCGTT
ACCAGGAGCGATCAGGGGCTTTATACTTGCGCGGCTTCTTCCGGACTCATGACAAAGAAAAACAGTACTTTCGTT
CGCGTCCATGAGAAAGATAAGACCCATACGTGCCCACCTTGTCCAGCTCCGGAACTTTTGGGCGGACCCAGCGTA
TTTCTTTTTCCTCCCAAGCCGAAAGACACACTGATGATATCAAGAACTCCTGAAGTTACATGTGTTGTGGTTGAC
GTCAGTCATGAAGACCCTGAGGTCAAGTTCAACTGGTATGTTGATGGAGTAGAGGTTCATAACGCGAAGACGAAG
CCACGAGAAGAGCAGTACAACAGCACGTATAGGGTGGTCAGTGTACTTACGGTACTCCACCAAGATTGGCTTAAC
GGAAAGGAATACAAGTGTAAAGTATCTAATAAGGCATTGCCGGCTCCGATTGAAAAGACAATCTCTAAGGCAAAA
GGCCAGCCGCGCGAGCCGCAAGTATATACATTGCCTCCCTCAAGAGATGAGCTTACCAAGAACCAGGTTTCTCTG
ACATGCCTTGTCAAAGGATTCTACCCTAGCGACATAGCCGTCGAATGGGAATCAAACGGTCAACCAGAGAACAAT
TATAAAACCACACCCCCGGTGCTTGATAGTGATGGCTCCTTTTTCCTGTATTCTAAGTTGACGGTGGATAAGAGT
CGGTGGCAACAGGGGAATGTATTTAGTTGCAGTGTGATGCACGAGGCTCTTCATAACCACTACACACAAAAATCT
TTGAGCTTGTCTCCAGGTTGA
SEQ ID NO: 11
AGCGACACCGGCCGCCCCTTCGTGGAGATGTACAGCGAGATCCCCGAGATCATCCACATGACCGAGGGCCGCGAG
CTGGTGATCCCCTGCCGCGTGACCAGCCCCAACATCACCGTGACCCTGAAGAAGTTCCCCCTGGACACCCTGATC
CCCGACGGCAAGCGCATCATCTGGGACAGCCGCAAGGGCTTCATCATCAGCAACGCCACCTACAAGGAGATCGGC
CTGCTGACCTGCGAGGCCACCGTGAACGGCCACCTGTACAAGACCAACTACCTGACCCACCGCCAGACCAACACC
ATCATCGACGTGGTGCTGAGCCCCAGCCACGGCATCGAGCTGAGCGTGGGCGAGAAGCTGGTGCTGAACTGCACC
GCCCGCACCGAGCTGAACGTGGGCATCGACTTCAACTGGGAGTACCCCAGCAGCAAGCACCAGCACAAGAAGCTG
GTGAACCGCGACCTGAAGACCCAGAGCGGCAGCGAGATGAAGAAGTTCCTGAGCACCCTGACCATCGACGGCGTG
ACCCGCAGCGACCAGGGCCTGTACACCTGCGCCGCCAGCAGCGGCCTGATGACCAAGAAGAACAGCACCTTCGTG
CGCGTGCACGAGAAGGACAAGACCCACACCTGCCCCCCCTGCCCCGCCCCCGAGCTGCTGGGCGGCCCCAGCGTG
TTCCTGTTCCCCCCCAAGCCCAAGGACACCCTGATGATCAGCCGCACCCCCGAGGTGACCTGCGTGGTGGTGGAC
GTGAGCCACGAGGACCCCGAGGTGAAGTTCAACTGGTACGTGGACGGCGTGGAGGTGCACAACGCCAAGACCAAG
CCCCGCGAGGAGCAGTACAACAGCACCTACCGCGTGGTGAGCGTGCTGACCGTGCTGCACCAGGACTGGCTGAAC
GGCAAGGAGTACAAGTGCAAGGTGAGCAACAAGGCCCTGCCCGCCCCCATCGAGAAGACCATCAGCAAGGCCAAG
GGCCAGCCCCGCGAGCCCCAGGTGTACACCCTGCCCCCCAGCCGCGACGAGCTGACCAAGAACCAGGTGAGCCTG
ACCTGCCTGGTGAAGGGCTTCTACCCCAGCGACATCGCCGTGGAGTGGGAGAGCAACGGCCAGCCCGAGAACAAC
TACAAGACCACCCCCCCCGTGCTGGACAGCGACGGCAGCTTCTTCCTGTACAGCAAGCTGACCGTGGACAAGAGC
CGCTGGCAGCAGGGCAACGTGTTCAGCTGCAGCGTGATGCACGAGGCCCTGCACAACCACTACACCCAGAAGAGC
CTGAGCCTGAGCCCCGGCTAA

Preferably, the polynucleotide encoding the aflibercept polypeptide is SEQ ID NO: 11 or a variant thereof.

In some embodiments, the nucleotide sequence encoding aflibercept has at least 75%, 80%, 85%, 86%, 87%, 88%, 89%, 90%, 91%, 92%, 93%, 94%, 95%, 96%, 97%, 98% or 99% identity to any of SEQ ID NO: 2-11, preferably SEQ ID NO: 11. Preferably, the protein encoded by the nucleotide sequence substantially retains a functional activity of the protein represented by SEQ ID NO: 1.

Ranibizumab is a monoclonal antibody fragment (Fab) against VEGFA. Suitably, the ranibizumab polypeptide may comprise heavy and light chain variable sequences shown as SEQ ID NO: 12 and 13, respectively. Complementarity determining regions (CDRs) are shown underlined in each of the sequences.

SEQ ID NO: 12
EVQLVESGGGLVQPGGSLRLSCAASGYDFTHYGMNWVRQAPGKGLEWVGW
INTYTGEPTYAADFKRRFTFSLDTSKSTAYLQMNSLRAEDTAVYYCAKYP
YYYGTSHWYFDVWGQGTLVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKKVEPKSCDKTHL
SEQ ID NO: 13
DIQLTQSPSSLSASVGDRVTITCSASQDISNYLNWYQQKPGKAPKVLIYF
TSSLHSGVPSRFSGSGSGTDFTLTISSLQPEDFATYYCQQYSTVPWTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

The ranibizumab heavy and light chain variable sequences may comprise polypeptides shown as SEQ ID NO: 12 or 13. The variants may have at least 75%, 80%, 85%, 86%, 87%, 88%, 89%, 90%, 91%, 92%, 93%, 94%, 95%, 96%, 97%, 98% or 99% identity to SEQ ID NO: 12 or 13. Preferably, the fragment or variant substantially retains a functional activity of the heavy and light chain variable sequences shown as SEQ ID NO: 12 and 13.

The ranibizumab heavy and light chain polypeptides may be encoded by any suitable polynucleotide sequences. The polynucleotide sequences encoding the heavy and light chains may be operably linked in an isolated polynucleotide. For example, the polynucleotide sequences encoding the heavy and light chains may be operably linked to the same promoter and be separated from each other by an internal ribosomal entry site (IRES) or a polynucleotide sequence encoding a self-cleaving polypeptide.

Examples of polynucleotide sequences which encode ranibizumab heavy and light chain polypeptides; respectively, are shown as SEQ ID NO: 14 and 15.

SEQ ID NO: 14
GAGGTGCAGCTGGTGGAGAGCGGCGGCGGCCTGGTGCAGCCCGGCGGCAG
CCTGCGCCTGAGCTGCGCCGCCAGCGGCTACGACTTCACCCACTACGGCA
TGAACTGGGTGCGCCAGGCCCCCGGCAAGGGCCTGGAGTGGGTGGGCTGG
ATCAACACCTACACCGGCGAGCCCACCTACGCCGCCGACTTCAAGCGCCG
CTTCACCTTCAGCCTGGACACCAGCAAGAGCACCGCCTACCTGCAGATGA
ACAGCCTGCGCGCCGAGGACACCGCCGTGTACTACTGCGCCAAGTACCCC
TACTACTACGGCACCAGCCACTGGTACTTCGACGTGTGGGGCCAGGGCAC
CCTGGTGACCGTGAGCAGCGCCAGCACCAAGGGCCCCAGCGTGTTCCCCC
TGGCCCCCAGCAGCAAGAGCACCAGCGGCGGCACCGCCGCCCTGGGCTGC
CTGGTGAAGGACTACTTCCCCGAGCCCGTGACCGTGAGCTGGAACAGCGG
CGCCCTGACCAGCGGCGTGCACACCTTCCCCGCCGTGCTGCAGAGCAGCG
GCCTGTACAGCCTGAGCAGCGTGGTGACCGTGCCCAGCAGCAGCCTGGGC
ACCCAGACCTACATCTGCAACGTGAACCACAAGCCCAGCAACACCAAGGT
GGACAAGAAGGTGGAGCCCAAGAGCTGCGACAAGACCCACCTG
SEQ ID NO: 15
GACATCCAGCTGACCCAGAGCCCCAGCAGCCTGAGCGCCAGCGTGGGCGA
CCGCGTGACCATCACCTGCAGCGCCAGCCAGGACATCAGCAACTACCTGA
ACTGGTACCAGCAGAAGCCCGGCAAGGCCCCCAAGGTGCTGATCTACTTC
ACCAGCAGCCTGCACAGCGGCGTGCCCAGCCGCTTCAGCGGCAGCGGCAG
CGGCACCGACTTCACCCTGACCATCAGCAGCCTGCAGCCCGAGGACTTCG
CCACCTACTACTGCCAGCAGTACAGCACCGTGCCCTGGACCTTCGGCCAG
GGCACCAAGGTGGAGATCAAGCGCACCGTGGCCGCCCCCAGCGTGTTCAT
CTTCCCCCCCAGCGACGAGCAGCTGAAGAGCGGCACCGCCAGCGTGGTGT
GCCTGCTGAACAACTTCTACCCCCGCGAGGCCAAGGTGCAGTGGAAGGTG
GACAACGCCCTGCAGAGCGGCAACAGCCAGGAGAGCGTGACCGAGCAGGA
CAGCAAGGACAGCACCTACAGCCTGAGCAGCACCCTGACCCTGAGCAAGG
CCGACTACGAGAAGCACAAGGTGTACGCCTGCGAGGTGACCCACCAGGGC
CTGAGCAGCCCCGTGACCAAGAGCTTCAACCGCGGCGAGTGC

Examples of isolated polynucleotides, and AAV vectors, encoding ranibizumab are provided by WO2019/164854, for example.

Bevacizumab is a monoclonal antibody against VEGFA. Suitably, the bevacizumab polypeptide sequence may comprise heavy and light chain sequences shown as SEQ ID NO: 16 and 17, respectively. Complementarity determining regions (CDRs) are shown underlined in each of the sequences.

SEQ ID NO: 16
EVQLVESGGGLVQPGGSLRLSCAASGYTFTNYGMNWVRQAPGKGLEWVGW
INTYTGEPTYAADFKRRFTFSLDTSKSTAYLQMNSLRAEDTAVYYCAKYP
HYYGSSHWYFDVWGQGTLVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLS
PGK
SEQ ID NO: 17
DIQMTQSPSSLSASVGDRVTITCSASQDISNYLNWYQQKPGKAPKVLIYF
TSSLHSGVPSRFSGSGSGTDFTLTISSLQPEDFATYYCQQYSTVPWTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

The bevacizumab heavy and light chain sequences may comprise polypeptides shown as SEQ ID NO: 16 or 17. The variants may have at least 75%, 80%, 85%, 86%, 87%, 88%, 89%, 90%, 91%, 92%, 93%, 94%, 95%, 96%, 97%, 98% or 99% identity to SEQ ID NO: 16 or 17. Preferably, the fragment or variant substantially retains a functional activity of the heavy and light chain sequences shown as SEQ ID NO: 16 and 17.

Examples of polynucleotide sequences which encode bevacizumab heavy and light chain polypeptides; respectively, are shown as SEQ ID NO: 18 and 19.

SEQ ID NO: 18
GAGGTGCAGCTGGTGGAGAGCGGCGGCGGCCTGGTGCAGCCCGGCGGCAG
CCTGCGCCTGAGCTGCGCCGCCAGCGGCTACACCTTCACCAACTACGGCA
TGAACTGGGTGCGCCAGGCCCCCGGCAAGGGCCTGGAGTGGGTGGGCTGG
ATCAACACCTACACCGGCGAGCCCACCTACGCCGCCGACTTCAAGCGCCG
CTTCACCTTCAGCCTGGACACCAGCAAGAGCACCGCCTACCTGCAGATGA
ACAGCCTGCGCGCCGAGGACACCGCCGTGTACTACTGCGCCAAGTACCCC
CACTACTACGGCAGCAGCCACTGGTACTTCGACGTGTGGGGCCAGGGCAC
CCTGGTGACCGTGAGCAGCGCCAGCACCAAGGGCCCCAGCGTGTTCCCCC
TGGCCCCCAGCAGCAAGAGCACCAGCGGCGGCACCGCCGCCCTGGGCTGC
CTGGTGAAGGACTACTTCCCCGAGCCCGTGACCGTGAGCTGGAACAGCGG
CGCCCTGACCAGCGGCGTGCACACCTTCCCCGCCGTGCTGCAGAGCAGCG
GCCTGTACAGCCTGAGCAGCGTGGTGACCGTGCCCAGCAGCAGCCTGGGC
ACCCAGACCTACATCTGCAACGTGAACCACAAGCCCAGCAACACCAAGGT
GGACAAGAAGGTGGAGCCCAAGAGCTGCGACAAGACCCACACCTGCCCCC
CCTGCCCCGCCCCCGAGCTGCTGGGCGGCCCCAGCGTGTTCCTGTTCCCC
CCCAAGCCCAAGGACACCCTGATGATCAGCCGCACCCCCGAGGTGACCTG
CGTGGTGGTGGACGTGAGCCACGAGGACCCCGAGGTGAAGTTCAACTGGT
ACGTGGACGGCGTGGAGGTGCACAACGCCAAGACCAAGCCCCGCGAGGAG
CAGTACAACAGCACCTACCGCGTGGTGAGCGTGCTGACCGTGCTGCACCA
GGACTGGCTGAACGGCAAGGAGTACAAGTGCAAGGTGAGCAACAAGGCCC
TGCCCGCCCCCATCGAGAAGACCATCAGCAAGGCCAAGGGCCAGCCCCGC
GAGCCCCAGGTGTACACCCTGCCCCCCAGCCGCGAGGAGATGACCAAGAA
CCAGGTGAGCCTGACCTGCCTGGTGAAGGGCTTCTACCCCAGCGACATCG
CCGTGGAGTGGGAGAGCAACGGCCAGCCCGAGAACAACTACAAGACCACC
CCCCCCGTGCTGGACAGCGACGGCAGCTTCTTCCTGTACAGCAAGCTGAC
CGTGGACAAGAGCCGCTGGCAGCAGGGCAACGTGTTCAGCTGCAGCGTGA
TGCACGAGGCCCTGCACAACCACTACACCCAGAAGAGCCTGAGCCTGAGC
CCCGGCAAG
SEQ ID NO: 19
GACATCCAGATGACCCAGAGCCCCAGCAGCCTGAGCGCCAGCGTGGGCGA
CCGCGTGACCATCACCTGCAGCGCCAGCCAGGACATCAGCAACTACCTGA
ACTGGTACCAGCAGAAGCCCGGCAAGGCCCCCAAGGTGCTGATCTACTTC
ACCAGCAGCCTGCACAGCGGCGTGCCCAGCCGCTTCAGCGGCAGCGGCAG
CGGCACCGACTTCACCCTGACCATCAGCAGCCTGCAGCCCGAGGACTTCG
CCACCTACTACTGCCAGCAGTACAGCACCGTGCCCTGGACCTTCGGCCAG
GGCACCAAGGTGGAGATCAAGCGCACCGTGGCCGCCCCCAGCGTGTTCAT
CTTCCCCCCCAGCGACGAGCAGCTGAAGAGCGGCACCGCCAGCGTGGTGT
GCCTGCTGAACAACTTCTACCCCCGCGAGGCCAAGGTGCAGTGGAAGGTG
GACAACGCCCTGCAGAGCGGCAACAGCCAGGAGAGCGTGACCGAGCAGGA
CAGCAAGGACAGCACCTACAGCCTGAGCAGCACCCTGACCCTGAGCAAGG
CCGACTACGAGAAGCACAAGGTGTACGCCTGCGAGGTGACCCACCAGGGC
CTGAGCAGCCCCGTGACCAAGAGCTTCAACCGCGGCGAGTGC

The bevacizumab heavy and light chain polypeptides may be encoded by any suitable polynucleotide sequences. The polynucleotide sequences encoding the heavy and light chains may be operably linked in an isolated polynucleotide. For example, the polynucleotide sequences encoding the heavy and light chains may be operably linked to the same promoter and be separated from each other by an internal ribosomal entry site (IRES) or a polynucleotide sequence encoding a self-cleaving polypeptide.

Pegaptanib is a RNA aptamer specific for the VEGF₍₁₆₅₎ isoform. A RNA sequence for pegatanib is shown as SEQ ID NO: 20.

| SEQ ID NO: 20 | |
|---|---|
| | GCGGAAUCAGUGAAUGCUUAUACAUCCGC |

The pegaptanib polynucleotide sequence may comprise a sequence shown as SEQ ID NO: 20. The variant may have at least 75%, 80%, 85%, 86%, 87%, 88%, 89%, 90%, 91%, 92%, 93%, 94%, 95%, 96%, 97%, 98% or 99% identity to SEQ ID NO: 20. Preferably, the fragment or variant substantially retains a functional activity of the polynucleotide shown as SEQ ID NO: 20.

An example polynucleotide sequence encoding pegaptanib is shown as SEQ ID NO: 21.

| SEQ ID NO: 21 | |
|---|---|
| | CGCCTTAGTCACTTACGAATATGTAGGCG |

Signal Peptide

The polynucleotide sequence encoding the anti-VEGF entity may further comprise a polynucleotide sequence encoding signal peptide to allow secretion of the anti-VEGF entity from a cell.

A signal peptide is a short peptide, commonly 5-30 amino acids long, typically present at the N-terminus of the majority of newly synthesized proteins that are destined towards the secretory pathway. These proteins include those that reside either inside certain organelles (for example, the endoplasmic reticulum, Golgi or endosomes), are secreted from the cell, and transmembrane proteins.

Signal peptides commonly contain a core sequence which is a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases.

The signal peptide is commonly positioned at the amino terminus of the molecule, although some carboxy-terminal signal peptides are known.

Signal sequences typically have a tripartite structure, consisting of a hydrophobic core region (h-region) flanked by an n- and c-region. The latter contains the signal peptidase (SPase) consensus cleavage site. Usually, signal sequences are cleaved off co-translationally, the resulting cleaved signal sequences are termed signal peptides.

Signal sequences can be detected or predicted using software techniques (see for example, http://www.predisi.de/). A large number of signal sequences are known, and are available in databases. For example, http://www.signalpeptide.de lists 2109 confirmed mammalian signal peptides in its database.

Suitably, the signal peptide may be a CFH signal peptide.

Illustrative polynucleotide sequences encoding a CFH signal peptide are shown as SEQ ID NO: 22-31.

SEQ ID NO: 22
ATGAGACTTCTAGCAAAGATTATTTGCCTTATGTTATGGGCTATTTGTGT
AGCA
SEQ ID NO: 23
ATGAGACTGCTGGCCAAGATCATCTGCCTGATGCTGTGGGCCATCTGCGT
GGCC
SEQ ID NO: 24
ATGAGACTGCTCGCCAAGATCATTTGCCTGATGCTGTGGGCCATCTGCGT
GGCC
SEQ ID NO: 25
ATGCGGCTGCTGGCCAAGATCATCTGCCTGATGCTGTGGGCCATCTGCGT
GGCA
SEQ ID NO: 26
ATGCGGCTGCTCGCCAAGATCATTTGCCTGATGCTGTGGGCCATCTGCGT
GGCA
SEQ ID NO: 27
ATGAGACTGCTCGCCAAGATCATCTGTCTGATGCTGTGGGCTATCTGTGT
CGCT
SEQ ID NO: 28
ATGAGACTGCTCGCCAAGATCATTTGTCTGATGCTGTGGGCTATCTGTGT
CGCT
SEQ ID NO: 29
ATGCGCTTGCTGGCGAAGATAATCTGCCTGATGTTGTGGGCCATATGCGT
AGCC
SEQ ID NO: 30
ATGCGCTTGCTGGCGAAGATAATCTGCCTGATGTTGTGGGCCATATGCGT
AGCC
SEQ ID NO: 31
ATGCGCCTGCTGGCCAAGATCATCTGCCTGATGCTGTGGGCCATCTGCGT
GGCC

Complement System

The complement system is an integral part of the humoral immune system and is involved in tissue inflammation, cell opsonisation, and cytolysis. It provides protection against microorganisms and mediates the clearance of exogenous and endogenous cellular debris from the host tissues.

The complement system cascade is comprised of three activation pathways. All of the pathways ultimately end in the central cleavage of C3 factor and in the generation of its active fragments C3a and C3b. C3a is the anaphylatoxin that triggers a range of chemotactic and proinflammatory responses, such as recruitment of inflammatory cells and increased microvasculature permeability, whereas C3b is responsible for opsonisation of foreign surfaces covalently attached to C3b. Opsonisation with activated C3 fragments (C3b and iC3b) fulfils three major functions: (i) cell debris elimination by phagocytic cells (e.g. macrophages or microglia) and the stimulation of the adaptive immune system (B and T cells); (ii) amplification of complement activation via the formation of a surface-bound C3 convertase; and (iii) assemblage of the C5 convertase.

Assemblage of the C5 convertase is responsible for C5 cleavage, which results in the formation of the cytolytic membrane attack complex (MAC) capable of generating perforations in the cell membrane, thereby promoting cell lysis and the elimination of unnecessary cells. Through all of these activities, the innate complement cascade supports and promotes the function of downstream mechanisms of the immune system that protect the integrity of the host tissue. Overall, complement system pathway activation results in a proinflammatory response, including MAC generation, which mediates cell lysis, the release of chemokines to attract inflammatory cells to the site of damage, and the enhancement of capillary permeability to promote extravasation of infiltrating leukocytes. Under physiological conditions, complement activation is effectively controlled by the coordinated action of soluble and membrane-associated complement regulatory molecules (CRMs). Soluble complement regulators, such as C1-inhibitor, anaphylatoxins inhibitor, C4b binding protein (C4BP), Complement Factor H (CFH), Complement Factor I (CFI), clusterin and vitronectin, restrict the action of complement in human tissues at multiple sites of the cascade reaction. In addition, each individual cell is protected against the attack of homologous complement by surface proteins, such as the Complement Receptor 1 (CR1, CD35), the membrane cofactor protein (MCP, CD46), and glycosylphosphatidylinositol-anchored proteins, such as decay-accelerating factor (CD55) or CD59 molecule. Of note, host cells and tissues that are inadequately protected from complement attack might be subjected to bystander cell lysis.

In some embodiments, the invention relates to the treatment or prevention of a complement-mediated disorder of the eye. For example, the complement-mediated disorder may be a disorder associated with a defect in alternative pathway regulation, and in particular with over-activity of the complement C3b feedback cycle and/or under-activity of the C3b breakdown cycle.

The present invention comprises the use of a negative complement regulator. A negative complement regulator may refer to an entity which restricts the action of complement at one or more sites of the cascade reaction. By way of example, the negative complement regulator may be C1-inhibitor, anaphylatoxins inhibitor, C4b binding protein (C4BP), Complement Factor H (CFH), Factor H like protein 1 (FHL1), Complement Factor I (CFI), clusterin or vitronectin. The negative complement regulator may also be a surface protein which is capable of protecting cells against the attack of homologous complement; for example Complement Receptor 1 (CR1, CD35), membrane cofactor protein (CD46), and glycosylphosphatidylinositol-anchored proteins, such as decay-accelerating factor (CD55) or CD59 molecule.

Suitably, a negative complement regulator may be an inhibitor of a component of the complement activation cascade, for example an inhibitor of the complement C3b feedback cycle

The negative complement regulator may be selected from CFI, FHL1, CFH, CR1 and MCP or a variant or fragment thereof.

The negative complement regulator may be selected from CFI, FHL1 and CFH or a variant or fragment thereof.

The negative complement regulator may be CFI or a variant or fragment thereof.

The negative complement regulator may be FHL1 or a variant or fragment thereof.

The negative complement regulator may be CFH or a variant or fragment thereof.

The negative complement regulator may be CR1 or a variant or fragment thereof.

The negative complement regulator may be MCP or a variant or fragment thereof.

Suitably, the product may comprise one or more negative complement regulators.

Suitably, the polynucleotide may encode one or more negative complement regulators.

In some embodiments, prior to administration of the product, polynucleotide, vector, cell or pharmaceutical composition of the invention, the subject has low levels (e.g. lower than normal levels) of Complement Factor I activity, for example low levels of Complement Factor I activity in the eye and/or low serum levels of Complement Factor I activity. The sub-normal level of Complement Factor I activity may be due to sub-normal expression of normally-functioning Complement Factor I, or at least partial (e.g. heterozygous) expression (at normal or sub-normal levels) of a non- or sub-functional variant of Complement Factor I. (Such a subject may carry one or more copies of an AMD-associated SNP, for example the subject may be homo- or heterozygous for one of the rare Complement Factor I variants discussed further below). Thus, the subject may have a low concentration (e.g. a lower than normal concentration) of Complement Factor I in the eye and/or serum. For a human subject, the normal level of Complement Factor I activity (C3b-inactivating and iC3b-degradation activity) may be equivalent to that provided by 30-40 μg/mL Complement Factor I in the serum of the subject. Thus, in a subject with low Complement Factor I activity, the Complement Factor I activity in the serum may correspond to less than 30 μg/mL and greater than 0 μg/mL Complement Factor I, such as 0-20 or 0-10 μg/mL (these being ranges of Complement Factor I serum concentration which may encompass a subject having a low Complement Factor I concentration).

Thus, the subject to be treated by the invention may suffer from a complement-mediated disorder of the eye such as AMD, or may be at risk of developing such a disorder. The AMD may by wet AMD and/or dry AMD. For example, the subject may be homozygous or heterozygous susceptible for one or more SNPs associated with the complement-mediated disorder.

In some embodiments, the subject is at risk of developing AMD. For example, the subject may be homozygous or heterozygous susceptible for one or more SNPs associated with AMD, for example rare mutations in Complement Factor I associated with advanced AMD which commonly result in reduced serum Complement Factor I levels (Kavanagh et al. (2015) Hum Mol Genet 24: 3861-3870). In particular the subject may carry one or two copies of one or more of the following rare Complement Factor I variants: rs144082872 (encoding P50A); 4:110687847 (encoding P64L); rs141853578 (encoding G119R); 4:110685721 (encoding V152M); 4:110682846 (encoding G162D); 4:110682801 (encoding N1771); rs146444258 (encoding A240G); rs182078921 (encoding G287R); rs41278047 (encoding K441R); and rs121964913 (encoding R474).

The invention may further comprise determining whether the subject is at risk of developing a complement-mediated disorder (for example, AMD), for example by determining whether the subject is homozygous or heterozygous susceptible for one or more SNPs associated with the complement-mediated disorder (for example, by determining whether the subject is homozygous or heterozygous for one or more of the rare Complement Factor I variants associated with AMD listed above).

Alternatively, the subject may have a normal level of endogenous Complement Factor I activity or concentration, for example in the eye and/or serum and/or may not carry a rare variant Complement Factor I allele.

In some embodiments, administration of the product, polynucleotide, vector, cell or pharmaceutical composition of the invention thereby increases the level of C3b-inactivating and iC3b-degradation activity in the eye of the subject. In other embodiments, administration of the product, polynucleotide, vector, cell or pharmaceutical composition of the invention thereby increases the level of C3b-inactivating and iC3b-degradation activity in the eye of the subject to a level that exceeds a normal level in the eye. More particularly, the level of C3b-inactivating and iC3b-degradation activity is increased in the RPE of the eye.

It will be appreciated that the C3b-inactivating and iC3b-degradation activity in the subject following provision of the product of the invention and/or expression of the Complement Factor I and Complement Factor H-like Protein 1 from the polynucleotide or vector of the invention may comprise C3b-inactivating and iC3b-degradation activity from the subject's endogenous Complement Factor I (i.e. the subject's Complement Factor I not provided by the product or produced by expression from the polynucleotide or vector), and C3b-inactivating and iC3b-degradation activity provided by the product of the invention or produced by expression from the polynucleotide or vector of the invention, such that the total level of C3b-inactivating and iC3b-degradation activity in the subject exceeds a normal level.

In some embodiments, the level of C3b-inactivating and iC3b-degradation activity in the subject, for example in the eye, is increased to a level that is at least 5%, 10%, 15%, 20% or 25% above the normal level.

In other embodiments, the level of C3b-inactivating and iC3b-degradation activity in the subject, for example in the eye, is increased to a level that is up to twice the normal level, or up to 80%, 60%, 40% or 20% above the normal level.

For example, the level of C3b-inactivating and iC3b-degradation activity in the subject, for example in the eye, may be increased to a level that is 5-100%, 5-80%, 5-60%, 5-40%, 5-20%, 10-100%, 10-80%, 10-60%, 10-40%, 10-20%, 15-100%, 15-80%, 15-60%, 15-40%, 15-20%, 20-100%, 20-80%, 20-60%, 20-40%, 25-100%, 25-80%, 25-60% or 25-40% above the normal level.

In some embodiments, administration of the product, polynucleotide, vector, cell or pharmaceutical composition of the invention does not detectably increase the level of C3b-inactivating and iC3b-degradation activity in the plasma/serum of the subject. In other embodiments, administration of the product, polynucleotide, vector, cell or pharmaceutical composition of the invention does not detectably increase the level of C3b-inactivating and iC3b-degradation activity in the plasma/serum of the subject to a level greater than the normal level.

In the foregoing section, except where obviously inapplicable, reference to Complement Factor I and C3b-inactivating and iC3b-degradation activity may be replaced with Complement Factor H or Complement Factor H-like Protein 1, and ability to act as a cofactor for the Complement Factor I mediated cleavage of C3b and to increase the rate of dissociation of C3 convertase and C5 convertase, respectively. In some embodiments, prior to administration of the product, polynucleotide, vector, cell or pharmaceutical composition of the invention, the subject has low levels (e.g. lower than normal levels) of Complement Factor H, for example low levels of Complement Factor H in the eye and/or low serum levels of Complement Factor H. For a human subject, the normal level of Complement Factor H may be about 200-500 μg/mL in the serum of the subject. Thus, in a subject with low levels of Complement Factor H, the levels in the serum may be less than 200 μg/mL and greater than 0 μg/mL, such as 0-100 μg/mL. Alternatively, the subject may have a normal level of endogenous Complement Factor H, for example in the eye and/or serum.

Complement Factor I (CFI)

Complement Factor I (Factor I, CFI), also known as C3b/C4b inactivator, is a protein that in humans is encoded by the CFI gene.

Complement Factor I is a serine protease that circulates in a zymogen-like state (Roversi et al. (2011) PNAS 108: 12839-12844) at a concentration of ˜35 μg/mL (Nilsson et al. (2011) Mol Immunol 48: 1611-1620). The Complement Factor I protein is a heavily N-glycosylated heterodimer consisting of two polypeptide chains linked by a single disulfide bond. The heavy chain (50 kDa) comprises an N-terminal region; an FI membrane attack complex (FIMAC) domain; a CD5 like-domain or scavenger receptor cysteine-rich (SRCR) domain; two low-density lipoprotein receptor (LDLr) domains; and a C-terminal region of unknown function that is a site of sequence variability across species (Roversi et al. (2011) PNAS 108: 12839-12844). The light chain (38 kDa) contains the serine protease (SP) domain with the conserved catalytic residues (Goldberger et al. (1987) J Biol Chem 262: 10065-10071).

Complement Factor I inactivates C3b by cleaving it into iC3b, C3d and C3dg and, in an analogous way, C4b into C4c and C4d. To properly perform its functions, Complement Factor I requires the presence of cofactor proteins such as C4b-Binding Protein (C4BP), Complement Factor H (CFH), Complement Receptor 1 (CR1/CD35) and Membrane Cofactor Protein (MCP/CD46) (Degn et al. (2011) Am J Hum Genet 88: 689-705).

iC3b is incapable of associating with Factor B, and thus cannot perpetuate amplification of the complement cascade or activation through the alternative pathway. Hence, once C3b has been cleaved to iC3b, neither alternative pathway initiation nor terminal complement cascade activation occurs.

iC3b is capable of providing a proinflammatory action by binding to, and activating, Complement Receptor 3 (CR3)(CD11b/CD18) on polymorphonuclear leukocytes (mostly neutrophils), NK cells and mononuclear phagocytes, such as macrophages.

Complement Factor I is capable of processing iC3b into C3dg via a protease activity requiring the cofactor, CR1. C3d,g is unable to bind to CR3. Since iC3b reacting with the complement receptor CR3 is a major mechanism by which complement activation gives rise to inflammation, the breakdown of iC3b to C3dg is essential for reducing complement-induced inflammation (Lachmann (2009) Adv. Immunol. 104: 115-149).

Complement Factor l's unique ability to both promote cleavage of C3b to iC3b as well as accelerate breakdown of iC3b—combined with its relatively low concentration in human serum, with implications for the amount required to be delivered for therapeutic efficacy—make it a particularly advantageous target.

In some embodiments, a Complement Factor I polypeptide is capable of cleaving C3b into an inactive degradation product. For example, the Complement Factor I polypeptide may be capable of cleaving C3b into iC3b.

In some embodiments, a Complement Factor I polypeptide is capable of processing iC3b into an inactive degradation product. For example, the Complement Factor I polypeptide may be capable of processing iC3b into C3dg.

In preferred embodiments, the Complement Factor I polypeptide is capable of cleaving C3b into iC3b and processing iC3b into C3dg.

Suitably, a fragment or variant of Complement Factor I may retain at least 50%, 60%, 70%, 80%, 90%, 95% or 100% of the C3b-inactivating and iC3b-degradation activity of native Complement Factor I.

The C3b-inactivating and iC3b-degradation activity of Complement Factor I, or a fragment or derivative thereof, may be determined using any suitable method known to the skilled person. For example, measurement of Complement Factor I proteolytic activity is described in Hsiung et al. (Biochem. J. (1982) 203: 293-298). Both haemolytic and conglutinating assays for CFI activity are described in Lachmann P J & Hobart M J (1978) “Complement Technology” in Handbook of Experimental Immunology 3rd edition Ed D M Weir Blackwells Scientific Publications Chapter 5A p 17. A more detailed description, also including a proteolytic assay, is given by Harrison R A (1996) in “Weir's Handbook of Experimental Immunology” 5th Edition Eds; Herzenberg Leonore A'Weir D M, Herzenberg Leonard A & Blackwell C Blackwells Scientific Publications Chapter 75 36-37. The conglutinating assay is highly sensitive and can be used for detecting both the first (double) clip converting fixed C3b to iC3b and acquiring reactivity with conglutinin; and for detecting the final clip to C3dg by starting with fixed iC3b and looking for the loss of reactivity with conglutinin. The haemolytic assay is used for the conversion of C3b to iC3b, and the proteolytic assay detects all the clips.

In some embodiments, the Complement Factor I is human Complement Factor I.

An example human Complement Factor I protein is the human Complement Factor I protein having the UniProtKB accession number P05156. This exemplified sequence is 583 amino acids in length (disclosed as SEQ ID NO: 32) of which amino acids 1 to 18 form a signal sequence.

In some embodiments, the amino acid sequence of Complement Factor I is SEQ ID NO: 32. In other embodiments, the amino acid sequence of Complement Factor I is the sequence disclosed as positions 19 to 583 of SEQ ID NO: 32. Preferably, variant or fragment substantially retains a functional activity of the protein represented by SEQ ID NO: 32

(SEQ ID NO: 32)
MKLLHVFLLFLCFHLRFCKVTYTSQEDLVEKKCLAKKYTHLSCDKVFCQP
WQRCIEGTCVCKLPYQCPKNGTAVCATNRRSFPTYCQQKSLECLHPGTKF
LNNGTCTAEGKFSVSLKHGNTDSEGIVEVKLVDQDKTMFICKSSWSMREA
NVACLDLGFQQGADTQRRFKLSDLSINSTECLHVHCRGLETSLAECTFTK
RRTMGYQDFADVVCYTQKADSPMDDFFQCVNGKYISQMKACDGINDCGDQ
SDELCCKACQGKGFHCKSGVCIPSQYQCNGEVDCITGEDEVGCAGFASVT
QEETEILTADMDAERRRIKSLLPKLSCGVKNRMHIRRKRIVGGKRAQLGD
LPWQVAIKDASGITCGGIYIGGCWILTAAHCLRASKTHRYQIWTTVVDWI
HPDLKRIVIEYVDRIIFHENYNAGTYQNDIALIEMKKDGNKKDCELPRSI
PACVPWSPYLFQPNDTCIVSGWGREKDNERVFSLQWGEVKLISNCSKFYG
NRFYEKEMECAGTYDGSIDACKGDSGGPLVCMDANNVTYVWGVVSWGENC
GKPEFPGVYTKVANYFDWISYHVGRPFISQYNV

In some embodiments, the amino acid sequence of Complement Factor I is SEQ ID NO: 33, which corresponds to NCBI Accession No. NP_000195. In other embodiments, the amino acid sequence of Complement Factor I is the sequence disclosed as positions 19 to 583 of SEQ ID NO: 33.

(SEQ ID NO: 33)
MKLLHVFLLFLCFHLRFCKVTYTSQEDLVEKKCLAKKYTHLSCDKVFCQP
WQRCIEGTCVCKLPYQCPKNGTAVCATNRRSFPTYCQQKSLECLHPGTKF
LNNGTCTAEGKFSVSLKHGNTDSEGIVEVKLVDQDKTMFICKSSWSMREA
NVACLDLGFQQGADTQRRFKLSDLSINSTECLHVHCRGLETSLAECTFTK
RRTMGYQDFADVVCYTQKADSPMDDFFQCVNGKYISQMKACDGINDCGDQ
SDELCCKACQGKGFHCKSGVCIPSQYQCNGEVDCITGEDEVGCAGFASVA
QEETEILTADMDAERRRIKSLLPKLSCGVKNRMHIRRKRIVGGKRAQLGD
LPWQVAIKDASGITCGGIYIGGCWILTAAHCLRASKTHRYQIWTTVVDWI
HPDLKRIVIEYVDRIIFHENYNAGTYQNDIALIEMKKDGNKKDCELPRSI
PACVPWSPYLFQPNDTCIVSGWGREKDNERVFSLQWGEVKLISNCSKFYG
NRFYEKEMECAGTYDGSIDACKGDSGGPLVCMDANNVTYVWGVVSWGENC
GKPEFPGVYTKVANYFDWISYHVGRPFISQYNV

An example wild type nucleotide sequence encoding Complement Factor I is the nucleotide sequence having the NCBI Accession No. NM_000204, disclosed herein as SEQ ID NO: 34.

(SEQ ID NO: 34)
ATGAAGCTTCTTCATGTTTTCCTGTTATTTCTGTGCTTCCACTTAAGGTT
TTGCAAGGTCACTTATACATCTCAAGAGGATCTGGTGGAGAAAAAGTGCT
TAGCAAAAAAATATACTCACCTCTCCTGCGATAAAGTCTTCTGCCAGCCA
TGGCAGAGATGCATTGAGGGCACCTGTGTTTGTAAACTACCGTATCAGTG
CCCAAAGAATGGCACTGCAGTGTGTGCAACTAACAGGAGAAGCTTCCCAA
CATACTGTCAACAAAAGAGTTTGGAATGTCTTCATCCAGGGACAAAGTTT
TTAAATAACGGAACATGCACAGCCGAAGGAAAGTTTAGTGTTTCCTTGAA
GCATGGAAATACAGATTCAGAGGGAATAGTTGAAGTAAAACTTGTGGACC
AAGATAAGACAATGTTCATATGCAAAAGCAGCTGGAGCATGAGGGAAGCC
AACGTGGCCTGCCTTGACCTTGGGTTTCAACAAGGTGCTGATACTCAAAG
AAGGTTTAAGTTGTCTGATCTCTCTATAAATTCCACTGAATGTCTACATG
TGCATTGCCGAGGATTAGAGACCAGTTTGGCTGAATGTACTTTTACTAAG
AGAAGAACTATGGGTTACCAGGATTTCGCTGATGTGGTTTGTTATACACA
GAAAGCAGATTCTCCAATGGATGACTTCTTTCAGTGTGTGAATGGGAAAT
ACATTTCTCAGATGAAAGCCTGTGATGGTATCAATGATTGTGGAGACCAA
AGTGATGAACTGTGTTGTAAAGCATGCCAAGGCAAAGGCTTCCATTGCAA
ATCGGGTGTTTGCATTCCAAGCCAGTATCAATGCAATGGTGAGGTGGACT
GCATTACAGGGGAAGATGAAGTTGGCTGTGCAGGCTTTGCATCTGTGGCT
CAAGAAGAAACAGAAATTTTGACTGCTGACATGGATGCAGAAAGAAGACG
GATAAAATCATTATTACCTAAACTATCTTGTGGAGTTAAAAACAGAATGC
ACATTCGAAGGAAACGAATTGTGGGAGGAAAGCGAGCACAACTGGGAGAC
CTCCCATGGCAGGTGGCAATTAAGGATGCCAGTGGAATCACCTGTGGGGG
AATTTATATTGGTGGCTGTTGGATTCTGACTGCTGCACATTGTCTCAGAG
CCAGTAAAACTCATCGTTACCAAATATGGACAACAGTAGTAGACTGGATA
CACCCCGACCTTAAACGTATAGTAATTGAATACGTGGATAGAATTATTTT
CCATGAAAACTACAATGCAGGCACTTACCAAAATGACATCGCTTTGATTG
AAATGAAAAAAGACGGAAACAAAAAAGATTGTGAGCTGCCTCGTTCCATC
CCTGCCTGTGTCCCCTGGTCTCCTTACCTATTCCAACCTAATGATACATG
CATCGTTTCTGGCTGGGGACGAGAAAAAGATAACGAAAGAGTCTTTTCAC
TTCAGTGGGGTGAAGTTAAACTAATAAGCAACTGCTCTAAGTTTTACGGA
AATCGTTTCTATGAAAAAGAAATGGAATGTGCAGGTACATATGATGGTTC
CATCGATGCCTGTAAAGGGGACTCTGGAGGCCCCTTAGTCTGTATGGATG
CCAACAATGTGACTTATGTCTGGGGTGTTGTGAGTTGGGGGGAAAACTGT
GGAAAACCAGAGTTCCCAGGTGTTTACACCAAAGTGGCCAATTATTTTGA
CTGGATTAGCTACCATGTAGGAAGGCCTTTTATTTCTCAGTACAATGTAT
AA

In some embodiments, the nucleotide sequences of Complement Factor I used in the invention are codon-optimised.

A preferred nucleotide sequence encoding Complement Factor I is the nucleotide sequence shown as SEQ ID NO: 35.

(SEQ ID NO: 35)
ATGAAACTGCTGCATGTCTTCCTCCTCTTCCTGTGCTTCCACCTCCGTTT
CTGTAAAGTCACCTACACTAGCCAGGAGGATCTGGTGGAGAAGAAATGCC
TGGCCAAGAAGTATACCCACCTGAGCTGCGACAAAGTGTTCTGCCAGCCC
TGGCAACGCTGCATTGAAGGTACTTGTGTGTGCAAGCTGCCCTACCAGTG
CCCCAAGAACGGCACGGCCGTGTGTGCCACCAACAGGAGGAGCTTCCCCA
CCTACTGCCAGCAGAAGAGCCTGGAATGCCTCCACCCTGGCACCAAGTTT
CTGAACAACGGGACCTGCACAGCCGAGGGGAAATTCAGCGTCTCCCTCAA
GCACGGCAATACAGACTCCGAGGGCATTGTGGAAGTGAAGCTGGTGGACC
AGGACAAGACCATGTTCATCTGCAAAAGCAGCTGGTCCATGCGGGAGGCC
AATGTCGCCTGCCTGGACCTGGGCTTCCAGCAGGGCGCTGATACACAGCG
CCGCTTTAAACTCAGTGACCTCAGCATCAACAGCACTGAGTGTCTGCACG
TGCACTGCCGGGGCCTGGAGACCAGCCTGGCTGAGTGCACCTTCACCAAG
CGCAGGACCATGGGCTACCAGGATTTTGCAGATGTGGTCTGCTACACCCA
GAAGGCAGACAGCCCCATGGATGACTTCTTCCAGTGTGTCAATGGCAAGT
ACATTTCCCAGATGAAGGCTTGTGACGGGATCAATGATTGCGGGGATCAG
AGCGATGAGCTCTGCTGCAAGGCCTGCCAAGGGAAGGGCTTTCACTGTAA
GTCTGGGGTGTGCATCCCTTCTCAGTATCAGTGCAACGGAGAGGTGGACT
GCATCACTGGGGAGGACGAGGTGGGCTGTGCTGGCTTCGCCTCTGTGGCC
CAGGAGGAGACAGAGATCCTCACAGCTGACATGGATGCAGAGCGGCGGCG
CATCAAGAGTCTGCTCCCAAAGCTCTCCTGCGGCGTTAAGAATCGCATGC
ACATCCGGAGGAAGCGGATCGTTGGAGGCAAACGGGCTCAGCTGGGGGAC
TTGCCGTGGCAGGTGGCCATCAAAGATGCCTCCGGAATCACCTGTGGTGG
CATCTACATCGGCGGCTGCTGGATCCTGACCGCCGCCCACTGCCTTCGGG
CCAGCAAGACTCACCGCTACCAGATCTGGACCACCGTGGTGGATTGGATT
CACCCCGACCTGAAGAGGATTGTCATTGAGTATGTCGACCGCATCATCTT
CCATGAAAACTACAATGCCGGGACGTATCAGAACGACATCGCCCTCATCG
AGATGAAGAAGGATGGGAACAAGAAGGACTGTGAGCTGCCTCGCTCCATC
CCCGCCTGTGTACCATGGTCTCCGTACCTGTTCCAGCCAAATGACACATG
CATCGTGAGCGGCTGGGGCCGCGAGAAAGACAACGAGAGGGTCTTCTCCC
TGCAGTGGGGTGAAGTCAAGCTGATCAGCAACTGCTCCAAGTTCTACGGC
AACCGCTTCTATGAGAAGGAGATGGAGTGCGCCGGCACCTATGACGGCAG
CATTGACGCGTGCAAGGGAGACAGTGGGGGCCCCCTGGTCTGCATGGACG
CCAACAATGTGACCTACGTGTGGGGAGTTGTGTCCTGGGGCGAGAACTGT
GGCAAGCCTGAGTTCCCGGGCGTGTACACAAAGGTGGCAAACTATTTTGA
CTGGATCTCCTATCACGTTGGCAGGCCCTTCATTTCACAGTACAACGTA

A further example codon-optimised nucleotide sequence encoding Complement Factor I is SEQ ID NO: 36.

(SEQ ID NO: 36)
ATGAAGCTGCTGCATGTCTTTCTGCTGTTTCTGTGCTTCCATCTGCGGTT
CTGTAAAGTGACCTATACTAGCCAGGAGGATCTGGTGGAGAAGAAGTGTC
TGGCCAAGAAGTACACACACCTGAGCTGCGACAAGGTGTTCTGTCAGCCT
TGGCAGCGGTGCATCGAGGGCACCTGCGTGTGCAAGCTGCCTTACCAGTG
CCCAAAGAACGGCACCGCCGTGTGCGCCACAAATCGGAGATCTTTTCCAA
CATATTGCCAGCAGAAGAGCCTGGAGTGTCTGCACCCCGGCACCAAGTTC
CTGAACAATGGCACCTGCACAGCCGAGGGCAAGTTTTCTGTGAGCCTGAA
GCACGGCAACACAGATAGCGAGGGCATCGTGGAGGTGAAGCTGGTGGACC
AGGATAAGACCATGTTCATCTGTAAGAGCTCCTGGTCCATGAGGGAGGCA
AACGTGGCATGCCTGGATCTGGGATTCCAGCAGGGAGCAGACACACAGAG
GCGCTTTAAGCTGTCCGACCTGTCTATCAATAGCACCGAGTGCCTGCACG
TGCACTGTAGGGGCCTGGAGACATCCCTGGCAGAGTGCACCTTCACAAAG
CGGAGAACCATGGGCTACCAGGACTTTGCCGACGTGGTGTGCTATACCCA
GAAGGCCGATAGCCCCATGGACGATTTCTTTCAGTGCGTGAACGGCAAGT
ATATCTCCCAGATGAAGGCCTGCGACGGCATCAATGACTGTGGCGATCAG
TCTGACGAGCTGTGCTGTAAGGCCTGTCAGGGCAAGGGCTTCCACTGCAA
GAGCGGCGTGTGCATCCCTTCCCAGTACCAGTGCAACGGCGAGGTGGATT
GTATCACAGGAGAGGACGAAGTGGGATGCGCAGGATTTGCATCTGTGGCA
CAGGAGGAGACAGAGATCCTGACAGCCGACATGGATGCCGAGAGGCGCCG
GATCAAGTCTCTGCTGCCTAAGCTGAGCTGTGGCGTGAAGAATCGGATGC
ACATCAGAAGGAAGCGCATCGTGGGAGGCAAGAGGGCACAGCTGGGCGAT
CTGCCATGGCAGGTGGCCATCAAGGACGCCTCTGGCATCACCTGCGGCGG
CATCTACATCGGAGGATGTTGGATCCTGACCGCAGCACACTGCCTGAGAG
CAAGCAAGACACACAGGTATCAGATCTGGACCACAGTGGTGGATTGGATC
CACCCAGACCTGAAGAGAATCGTGATCGAGTACGTGGATAGGATCATCTT
TCACGAGAACTACAATGCCGGCACATATCAGAACGACATCGCCCTGATCG
AGATGAAGAAGGATGGCAATAAGAAGGACTGTGAGCTGCCCAGATCCATC
CCTGCATGCGTGCCATGGAGCCCCTATCTGTTCCAGCCCAACGATACCTG
CATCGTGTCCGGATGGGGAAGGGAGAAGGACAATGAGCGGGTGTTTTCTC
TGCAGTGGGGCGAGGTGAAGCTGATCTCCAACTGTTCTAAGTTCTACGGC
AATAGGTTTTATGAGAAGGAGATGGAGTGCGCCGGCACCTACGATGGCAG
CATCGACGCCTGTAAGGGCGATTCCGGAGGACCACTGGTGTGCATGGACG
CAAACAATGTGACATACGTGTGGGGAGTGGTGTCCTGGGGAGAGAACTGC
GGCAAGCCAGAGTTCCCCGGCGTATATACCAAGGTGGCCAATTATTTTGA
TTGGATTTCCTACCACGTCGGCAGGCCCTTTATTTCCCAGTATAATGTCT
AA

In some embodiments, the nucleotide sequence encoding Complement Factor I has at least 75%, 80%, 85%, 86%, 87%, 88%, 89%, 90%, 91%, 92%, 93%, 94%, 95%, 96%, 97%, 98% or 99% identity to SEQ ID NO: 34, 35 or 36, preferably SEQ ID NO: 35. Preferably, the protein encoded by the nucleotide sequence substantially retains a functional activity of the protein represented by SEQ ID NO: 34, 35 or 36.

In other embodiments, the nucleotide sequence encoding Complement Factor I is SEQ ID NO: 34, 35 or 36, preferably SEQ ID NO: 35.

In other embodiments, the nucleotide sequence encoding Complement Factor I has at least 75%, 80%, 85%, 90%, 91%, 92%, 93%, 94%, 95%, 96%, 97%, 98% or 99% identity to positions 55 to 1752 of SEQ ID NO: 34, 35 or 36, preferably SEQ ID NO: 35. Preferably, the protein encoded by the nucleotide sequence substantially retains a functional activity of the protein represented by SEQ ID NO: 32 or 33.

In other embodiments, the nucleotide sequence encoding Complement Factor I is positions 55 to 1752 of SEQ ID NO: 34, 35 or 36, preferably SEQ ID NO: 35.

In other embodiments, the nucleotide sequence encoding Complement Factor I encodes an amino acid sequence that has at least 75%, 80%, 85% 90%, 95%, 96%, 97%, 98% or 99% identity to SEQ ID NO: 32 or 33 Preferably, wherein the amino acid sequence substantially retains a functional activity of the protein represented by SEQ ID NO: 32 or 33.

In other embodiments, the nucleotide sequence encoding Complement Factor I encodes the amino acid sequence SEQ ID NO: 32 or 33.

In other embodiment, the nucleotide sequence encoding Complement Factor I encodes an amino acid sequence that has at least 75%, 80%, 85%, 90%, 95%, 96%, 97%, 98%, 99% identity to positions 19 to 583 of SEQ ID NO: 32 or 33. Preferably, wherein the amino acid sequence substantially retains a functional activity of the protein represented by SEQ ID NO: 32 or 33.

In other embodiments, the nucleotide sequence encoding Complement Factor I encodes the amino acid sequence of positions 19 to 583 of SEQ ID NO: 32 or 33.

An advantage of the invention is that Complement Factor I is particularly difficult to prepare in the form of a purified protein. Accordingly, the inventors have devised a way of modulating the complement system, for example to enable treatments of age-related macular degeneration (AMD), by administering Complement Factor I in the form of an AAV vector comprising a Complement Factor I-encoding nucleotide sequence. The AAV vector may be administered to a site of interest, for example the eye, to enable in situ translation of the Complement Factor I polypeptide.

Complement Factor H (CFH)

Complement Factor H (Factor H, CFH) is a complement control protein.

Complement Factor H is a large (155 kDa), soluble glycoprotein that is present in human plasma at a typical concentration of 200-300 μg/mL (Hakobyan et al. (2008) 49(5): 1983-90). The principal function of Complement Factor H is to regulate the alternative pathway of the complement system.

Complement Factor H provides cofactor activity for the Complement Factor I-mediated cleavage of C3b. Complement Factor H also increases the rate of dissociation of the C3bBb complex (C3 convertase) and the (C3b)NBB complex (C5 convertase) and thereby reduces the activity of the alternative complement pathway.

Complement Factor H is made up of 20 complement control protein (CCP) modules (also referred to as Short Consensus Repeats or sushi domains) connected to one another by short linkers (of between three and eight amino acid residues) and arranged in an extended head to tail fashion. Each of the CCP modules consists of around 60 amino acids with four cysteine residues disulfide bonded in a 1-3 2-4 arrangement, and a hydrophobic core built around an almost invariant tryptophan residue. The CCP modules are numbered from 1-20 (from the N-terminus of the protein). CCPs 1-4 and CCPs 19-20 engage with C3b while CCPs 7 and CCPs 19-20 bind to GAGs and sialic acid (Schmidt et al. (2008) Journal of Immunology 181: 2610-2619).

It has been shown that gene therapy using Complement Factor H can ameliorate induced AMD-like pathology in mice (Cashman et al. (2015) J. Gene Med. 17: 229-243). Mice were co-injected subretinally with: (i) an adenoviral vector expressing complement component C3, which had previously been shown to recapitulate many pathological features of human AMD; and (ii) an adenoviral vector expressing Complement Factor H. Relative to control animals receiving GFP instead of Complement Factor H, the Complement Factor H-transduced mice showed 91% reduction in endothelial cell proliferation and 69% attenuation of RPE atrophy. Electroretinography showed improved retinal function in mice receiving Complement Factor H, and immunocytochemistry of rhodopsin and RPE65 was consistent with the rescue of photoreceptors and RPE in such animals.

In some embodiments, a Complement Factor H polypeptide or a fragment or variant thereof is capable of acting as a cofactor for the Complement Factor I-mediated cleavage of C3b. In some embodiments, a Complement Factor H polypeptide or a fragment or variant thereof is capable of increasing the rate of dissociation of C3 convertase and C5 convertase.

In preferred embodiments, a Complement Factor H polypeptide or a fragment or variant thereof is capable of acting as a cofactor for the Complement Factor I-mediated cleavage of C3b and increasing the rate of dissociation of C3 convertase and C5 convertase.

The cofactor activity and dissociation acceleration activity of Complement Factor H, or a fragment or derivative thereof, may be determined using any suitable method known to the skilled person. For example, measurement of the cofactor activity of Complement Factor H is described in Senchez-Corral, P., et al., 2002. The American Journal of Human Genetics, 71(6), pp. 1285-1295 and measurement of the dissociation acceleration activity of Complement Factor H is described in Wong, E. K., et al., 2014. Journal of the American Society of Nephrology, 25(11), pp. 2425-2433.

In some embodiments, the Complement Factor H is human Complement Factor H.

An example human Complement Factor H protein is the human Complement Factor H protein having the UniProtKB accession number P08603. This exemplified sequence is 1231 amino acids in length (disclosed as SEQ ID NO: 37) of which amino acids 1 to 18 form a signal sequence.

In some embodiments, the amino acid sequence of Complement Factor H is SEQ ID NO: 37. In other embodiments, the amino acid sequence of Complement Factor H is positions 19 to 1231 of SEQ ID NO: 37. Preferably, variant or fragment substantially retains a functional activity of the protein represented by SEQ ID NO: 37

(SEQ ID NO: 37)
MRLLAKIICLMLWAICVAEDCNELPPRRNTEILTGSWSDQTYPEGTQAI
YKCRPGYRSLGNVIMVCRKGEWVALNPLRKCQKRPCGHPGDTPFGTFTL
TGGNVFEYGVKAVYTCNEGYOLLGEINYRECDTDGWTNDIPICEVVKCL
PVTAPENGKIVSSAMEPDREYHFGQAVRFVCNSGYKIEGDEEMHCSDDG
FWSKEKPKCVEISCKSPDVINGSPISQKIIYKENERFQYKCNMGYEYSE
RGDAVCTESGWRPLPSCEEKSCDNPYIPNGDYSPLRIKHRTGDEITYQC
RNGFYPATRGNTAKCTSTGWIPAPRCTLKPCDYPDIKHGGLYHENMRRP
YFPVAVGKYYSYYCDEHFETPSGSYWDHIHCTQDGWSPAVPCLRKCYFP
YLENGYNQNYGRKFVQGKSIDVACHPGYALPKAQTTVTCMENGWSPTPR
CIRVKTCSKSSIDIENGFISESQYTYALKEKAKYQCKLGYVTADGETSG
SITCGKDGWSAQPTCIKSCDIPVFMNARTKNDFTWFKLNDTLDYECHDG
YESNTGSTTGSIVCGYNGWSDLPICYERECELPKIDVHLVPDRKKDQYK
VGEVLKFSCKPGFTIVGPNSVQCYHFGLSPDLPICKEQVQSCGPPPELL
NGNVKEKTKEEYGHSEVVEYYCNPRFLMKGPNKIQCVDGEWTTLPVCIV
EESTCGDIPELEHGWAQLSSPPYYYGDSVEFNCSESFTMIGHRSITCIH
GVWTQLPQCVAIDKLKKCKSSNLIILEEHLKNKKEFDHNSNIRYRCRGK
EGWIHTVCINGRWDPEVNCSMAQIQLCPPPPQIPNSHNMTTTLNYRDGE
KVSVLCQENYLIQEGEEITCKDGRWQSIPLCVEKIPCSQPPQIEHGTIN
SSRSSQESYAHGTKLSYTCEGGFRISEENETTCYMGKWSSPPQCEGLPC
KSPPEISHGVVAHMSDSYQYGEEVTYKCFEGFGIDGPAIAKCLGEKWSH
PPSCIKTDCLSLPSFENAIPMGEKKDVYKAGEQVTYTCATYYKMDGASN
VTCINSRWTGRPTCRDTSCVNPPTVQNAYIVSRQMSKYPSGERVRYQCR
SPYEMFGDEEVMCLNGNWTEPPQCKDSTGKCGPPPPIDNGDITSFPLSV
YAPASSVEYQCQNLYQLEGNKRITCRNGQWSEPPKCLHPCVISREIMEN
YNIALRWTAKQKLYSRTGESVEFVCKRGYRLSSRSHTLRTTCWDGKLEY
PTCAKR

An example nucleotide sequence encoding Complement Factor H is the nucleotide sequence having the NCBI Accession No. NM_000186.

In some embodiments, the nucleotide sequence encoding Complement Factor H is SEQ ID NO: 38.

(SEQ ID NO: 38)
ATGAGACTTCTAGCAAAGATTATTTGCCTTATGTTATGGGCTATTTGTGTAGCAGAAGATTGCAATGAACTT
CCTCCAAGAAGAAATACAGAAATTCTGACAGGTTCCTGGTCTGACCAAACATATCCAGAAGGCACCCAGGCT
ATCTATAAATGCCGCCCTGGATATAGATCTCTTGGAAATGTAATAATGGTATGCAGGAAGGGAGAATGGGTT
GCTCTTAATCCATTAAGGAAATGTCAGAAAAGGCCCTGTGGACATCCTGGAGATACTCCTTTTGGTACTTTT
ACCCTTACAGGAGGAAATGTGTTTGAATATGGTGTAAAAGCTGTGTATACATGTAATGAGGGGTATCAATTG
CTAGGTGAGATTAATTACCGTGAATGTGACACAGATGGATGGACCAATGATATTCCTATATGTGAAGTTGTG
AAGTGTTTACCAGTGACAGCACCAGAGAATGGAAAAATTGTCAGTAGTGCAATGGAACCAGATCGGGAATAC
CATTTTGGACAAGCAGTACGGTTTGTATGTAACTCAGGCTACAAGATTGAAGGAGATGAAGAAATGCATTGT
TCAGACGATGGTTTTTGGAGTAAAGAGAAACCAAAGTGTGTGGAAATTTCATGCAAATCCCCAGATGTTATA
AATGGATCTCCTATATCTCAGAAGATTATTTATAAGGAGAATGAACGATTTCAATATAAATGTAACATGGGT
TATGAATACAGTGAAAGAGGAGATGCTGTATGCACTGAATCTGGATGGCGTCCGTTGCCTTCATGTGAAGAA
AAATCATGTGATAATCCTTATATTCCAAATGGTGACTACTCACCTTTAAGGATTAAACACAGAACTGGAGAT
GAAATCACGTACCAGTGTAGAAATGGTTTTTATCCTGCAACCCGGGGAAATACAGCAAAATGCACAAGTACT
GGCTGGATACCTGCTCCGAGATGTACCTTGAAACCTTGTGATTATCCAGACATTAAACATGGAGGTCTATAT
CATGAGAATATGCGTAGACCATACTTTCCAGTAGCTGTAGGAAAATATTACTCCTATTACTGTGATGAACAT
TTTGAGACTCCGTCAGGAAGTTACTGGGATCACATTCATTGCACACAAGATGGATGGTCGCCAGCAGTACCA
TGCCTCAGAAAATGTTATTTTCCTTATTTGGAAAATGGATATAATCAAAATCATGGAAGAAAGTTTGTACAG
GGTAAATCTATAGACGTTGCCTGCCATCCTGGCTACGCTCTTCCAAAAGCGCAGACCACAGTTACATGTATG
GAGAATGGCTGGTCTCCTACTCCCAGATGCATCCGTGTCAAAACATGTTCCAAATCAAGTATAGATATTGAG
AATGGGTTTATTTCTGAATCTCAGTATACATATGCCTTAAAAGAAAAAGCGAAATATCAATGCAAACTAGGA
TATGTAACAGCAGATGGTGAAACATCAGGATCAATTACATGTGGGAAAGATGGATGGTCAGCTCAACCCACG
TGCATTAAATCTTGTGATATCCCAGTATTTATGAATGCCAGAACTAAAAATGACTTCACATGGTTTAAGCTG
AATGACACATTGGACTATGAATGCCATGATGGTTATGAAAGCAATACTGGAAGCACCACTGGTTCCATAGTG
TGTGGTTACAATGGTTGGTCTGATTTACCCATATGTTATGAAAGAGAATGCGAACTTCCTAAAATAGATGTA
CACTTAGTTCCTGATCGCAAGAAAGACCAGTATAAAGTTGGAGAGGTGTTGAAATTCTCCTGCAAACCAGGA
TTTACAATAGTTGGACCTAATTCCGTTCAGTGCTACCACTTTGGATTGTCTCCTGACCTCCCAATATGTAAA
GAGCAAGTACAATCATGTGGTCCACCTCCTGAACTCCTCAATGGGAATGTTAAGGAAAAAACGAAAGAAGAA
TATGGACACAGTGAAGTGGTGGAATATTATTGCAATCCTAGATTTCTAATGAAGGGACCTAATAAAATTCAA
TGTGTTGATGGAGAGTGGACAACTTTACCAGTGTGTATTGTGGAGGAGAGTACCTGTGGAGATATACCTGAA
CTTGAACATGGCTGGGCCCAGCTTTCTTCCCCTCCTTATTACTATGGAGATTCAGTGGAATTCAATTGCTCA
GAATCATTTACAATGATTGGACACAGATCAATTACGTGTATTCATGGAGTATGGACCCAACTTCCCCAGTGT
GTGGCAATAGATAAACTTAAGAAGTGCAAATCATCAAATTTAATTATACTTGAGGAACATTTAAAAAACAAG
AAGGAATTCGATCATAATTCTAACATAAGGTACAGATGTAGAGGAAAAGAAGGATGGATACACACAGTCTGC
ATAAATGGAAGATGGGATCCAGAAGTGAACTGCTCAATGGCACAAATACAATTATGCCCACCTCCACCTCAG
ATTCCCAATTCTCACAATATGACAACCACACTGAATTATCGGGATGGAGAAAAAGTATCTGTTCTTTGCCAA
GAAAATTATCTAATTCAGGAAGGAGAAGAAATTACATGCAAAGATGGAAGATGGCAGTCAATACCACTCTGT
GTTGAAAAAATTCCATGTTCACAACCACCTCAGATAGAACACGGAACCATTAATTCATCCAGGTCTTCACAA
GAAAGTTATGCACATGGGACTAAATTGAGTTATACTTGTGAGGGTGGTTTCAGGATATCTGAAGAAAATGAA
ACAACATGCTACATGGGAAAATGGAGTTCTCCACCTCAGTGTGAAGGCCTTCCTTGTAAATCTCCACCTGAG
ATTTCTCATGGTGTTGTAGCTCACATGTCAGACAGTTATCAGTATGGAGAAGAAGTTACGTACAAATGTTTT
GAAGGTTTTGGAATTGATGGGCCTGCAATTGCAAAATGCTTAGGAGAAAAATGGTCTCACCCTCCATCATGC
ATAAAAACAGATTGTCTCAGTTTACCTAGCTTTGAAAATGCCATACCCATGGGAGAGAAGAAGGATGTGTAT
AAGGCGGGTGAGCAAGTGACTTACACTTGTGCAACATATTACAAAATGGATGGAGCCAGTAATGTAACATGC
ATTAATAGCAGATGGACAGGAAGGCCAACATGCAGAGACACCTCCTGTGTGAATCCGCCCACAGTACAAAAT
GCTTATATAGTGTCGAGACAGATGAGTAAATATCCATCTGGTGAGAGAGTACGTTATCAATGTAGGAGCCCT
TATGAAATGTTTGGGGATGAAGAAGTGATGTGTTTAAATGGAAACTGGACGGAACCACCTCAATGCAAAGAT
TCTACAGGAAAATGTGGGCCCCCTCCACCTATTGACAATGGGGACATTACTTCATTCCCGTTGTCAGTATAT
GCTCCAGCTTCATCAGTTGAGTACCAATGCCAGAACTTGTATCAACTTGAGGGTAACAAGCGAATAACATGT
AGAAATGGACAATGGTCAGAACCACCAAAATGCTTACATCCGTGTGTAATATCCCGAGAAATTATGGAAAAT
TATAACATAGCATTAAGGTGGACAGCCAAACAGAAGCTTTATTCGAGAACAGGTGAATCAGTTGAATTTGTG
TGTAAACGGGGATATCGTCTTTCATCACGTTCTCACACATTGCGAACAACATGTTGGGATGGGAAACTGGAG
TATCCAACTTGTGCAAAAAGATAG

In some embodiments, the nucleotide sequence encoding Complement Factor H has at least 75%, 80%, 85%, 90%, 95%, 96%, 97%, 98% or 99% identity to SEQ ID NO: 38. Preferably, wherein the protein encoded by the nucleotide sequence substantially retains a functional activity of the protein represented by SEQ ID NO: 37.

In other embodiments, the nucleotide sequence encoding Complement Factor H is SEQ ID NO: 38.

In other embodiments, the nucleotide sequence encoding Complement Factor H has at least 75%, 80%, 85% 90%, 95%, 96%, 97%, 98% or 99% identity to positions 55 to 3696 of SEQ ID NO: 38. Preferably, wherein the protein encoded by the nucleotide sequence substantially retains a functional activity of the protein represented by SEQ ID NO: 37.

In other embodiments, the nucleotide sequence encoding Complement Factor H is positions 55 to 3696 of SEQ ID NO: 38.

In other embodiments, the nucleotide sequence encoding Complement Factor H encodes an amino acid sequence that has at least 75%, 80%, 85% 90%, 95%, 96%, 97%, 98% or 99% identity to SEQ ID NO: 37. Preferably, wherein the amino acid sequence substantially retains a functional activity of the protein represented by SEQ ID NO: 37.

In other embodiments, the nucleotide sequence encoding Complement Factor H encodes the amino acid sequence SEQ ID NO: 37.

In other embodiment, the nucleotide sequence encoding Complement Factor H encodes an amino acid sequence that has at least 75%, 80%, 85%, 90%, 95%, 96%, 97%, 98%, 99% identity to positions 19 to 1231 of SEQ ID NO: 37. Preferably, wherein the amino acid sequence substantially retains a functional activity of the protein represented by SEQ ID NO: 37.

In other embodiments, the nucleotide sequence encoding Complement Factor H encodes the amino acid sequence of positions 19 to 1231 of SEQ ID NO: 37.

Complement Factor H-Like Protein 1 (FHL1)

Complement Factor H-like Protein 1 (FHL1) is a splice variant of Complement Factor H that contains the first 7 CCPs of Complement Factor H followed by a four amino acid carboxy-terminal tail (Clark, S. J. et al. (2015) J Clin Med 4: 18-31). The regulatory activity of FHL1 has been shown to be comparable to Complement Factor H (Mannes, M., et al., 2020. Frontiers in Immunology, 11; 596415).

In some embodiments, a FHL1 polypeptide or a fragment or variant thereof is capable of acting as a cofactor for the Complement Factor I-mediated cleavage of C3b. In some embodiments, a FHL1 polypeptide or a fragment or variant thereof is capable of increasing the rate of dissociation of C3 convertase and C5 convertase.

In preferred embodiments, a FHL1 polypeptide or a fragment or variant thereof is capable of acting as a cofactor for the Complement Factor I-mediated cleavage of C3b and increasing the rate of dissociation of C3 convertase and C5 convertase.

The cofactor activity and dissociation acceleration activity of a FHL1 polypeptide, or a fragment or derivative thereof, may be determined using any suitable method known to the skilled person. For example, those assays described above for Complement Factor H or any assay described in Mannes, M., et al., 2020. Frontiers in Immunology, 11; 596415.

In some embodiments, the FHL1 is human FHL1.

In some embodiments, the amino acid sequence of FHL1 is SEQ ID NO: 39. Preferably, variant or fragment substantially retains a functional activity of the protein represented by SEQ ID NO: 39. Suitably, a fragment or variant of FHL1 may retain at least 50%, 60%, 70%, 80%, 90%, 95% or 100% of the cofactor activity and/or dissociation acceleration activity of the protein represented by SEQ ID NO: 39.

(SEQ ID NO: 39)
MRLLAKIICLMLWAICVAEDCNELPPRRNTEILTGSWSDQTYPEGTQAI
YKCRPGYRSLGNIIMVCRKGEWVALNPLRKCQKRPCGHPGDTPFGTFTL
TGGNVFEYGVKAVYTCNEGYQLLGEINYRECDTDGWTNDIPICEVVKCL
PVTAPENGKIVSSAMEPDREYHFGQAVRFVCNSGYKIEGDEEMHCSDDG
FWSKEKPKCVEISCKSPDVINGSPISQKIIYKENERFQYKCNMGYEYSE
RGDAVCTESGWRPLPSCEEKSCDNPYIPNGDYSPLRIKHRTGDEITYQC
RNGFYPATRGNTAKCTSTGWIPAPRCTLKPCDYPDIKHGGLYHENMRRP
YFPVAVGKYYSYYCDEHFETPSGSYWDHIHCTQDGWSPAVPCLRKCYFP
YLENGYNQNYGRKFVQGKSIDVACHPGYALPKAQTTVTCMENGWSPTPR
CIRVSFTL

An example nucleotide sequence encoding FHL1 is:

(SEQ ID NO: 40)
ATGAGACTTCTAGCAAAGATTATTTGCCTTATGTTATGGGCTATTTGTGTAGCAGAAGATTGCAATGAACTT
CCTCCAAGAAGAAATACAGAAATTCTGACAGGTTCCTGGTCTGACCAAACATATCCAGAAGGCACCCAGGCT
ATCTATAAATGCCGCCCTGGATATAGATCTCTTGGAAATATAATAATGGTATGCAGGAAGGGAGAATGGGTT
GCTCTTAATCCATTAAGGAAATGTCAGAAAAGGCCCTGTGGACATCCTGGAGATACTCCTTTTGGTACTTTT
ACCCTTACAGGAGGAAATGTGTTTGAATATGGTGTAAAAGCTGTGTATACATGTAATGAGGGGTATCAATTG
CTAGGTGAGATTAATTACCGTGAATGTGACACAGATGGATGGACCAATGATATTCCTATATGTGAAGTTGTG
AAGTGTTTACCAGTGACAGCACCAGAGAATGGAAAAATTGTCAGTAGTGCAATGGAACCAGATCGGGAATAC
CATTTTGGACAAGCAGTACGGTTTGTATGTAACTCAGGCTACAAGATTGAAGGAGATGAAGAAATGCATTGT
TCAGACGATGGTTTTTGGAGTAAAGAGAAACCAAAGTGTGTGGAAATTTCATGCAAATCCCCAGATGTTATA
AATGGATCTCCTATATCTCAGAAGATTATTTATAAGGAGAATGAACGATTTCAATATAAATGTAACATGGGT
TATGAATACAGTGAAAGAGGAGATGCTGTATGCACTGAATCTGGATGGCGTCCGTTGCCTTCATGTGAAGAA
AAATCATGTGATAATCCTTATATTCCAAATGGTGACTACTCACCTTTAAGGATTAAACACAGAACTGGAGAT
GAAATCACGTACCAGTGTAGAAATGGTTTTTATCCTGCAACCCGGGGAAATACAGCaAAATGCACAAGTACT
GGCTGGATACCTGCTCCGAGATGTACCTTGAAACCTTGTGATTATCCAGACATTAAACATGGAGGTCTATAT
CATGAGAATATGCGTAGACCATACTTTCCAGTAGCTGTAGGAAAATATTACTCCTATTACTGTGATGAACAT
TTTGAGACTCCGTCAGGAAGTTACTGGGATCACATTCATTGCACACAAGATGGATGGTCGCCAGCAGTACCA
TGCCTCAGAAAATGTTATTTTCCTTATTTGGAAAATGGATATAATCAAAATTATGGAAGAAAGTTTGTACAG
GGTAAATCTATAGACGTTGCCTGCCATCCTGGCTACGCTCTTCCAAAAGCGCAGACCACAGTTACATGTATG
GAGAATGGCTGGTCTCCTACTCCCAGATGCATCCGTGTCAGCTTTACCCTCTGA

The nucleotide sequences of FHL1 used in the invention are preferably codon optimised.

A preferred nucleotide sequence encoding FHL1 is SEQ ID NO: 41.

(SEQ ID NO: 41)
ATGCGCCTCCTGGCCAAGATCATCTGCCTCATGCTGTGGGCCATCTGCGTGGCTGAGGACTGCAATGAGCTG
CCGCCCAGGAGGAACACAGAGATCCTGACAGGGAGCTGGTCTGACCAGACCTACCCTGAGGGCACCCAGGCG
ATCTACAAGTGCCGGCCGGGCTACAGGAGCCTGGGGAACATCATCATGGTGTGTAGAAAGGGCGAATGGGTG
GCCCTCAACCCCCTGAGGAAGTGCCAGAAGCGGCCCTGTGGCCACCCCGGGGACACACCCTTCGGGACCTTC
ACCCTGACCGGCGGCAATGTGTTTGAGTACGGCGTGAAGGCTGTCTACACATGCAACGAGGGGTACCAGCTG
CTGGGCGAGATTAACTACCGGGAGTGTGACACCGATGGGTGGACCAACGACATTCCCATCTGTGAGGTGGTC
AAGTGTCTCCCCGTGACAGCCCCAGAAAATGGCAAAATCGTGAGCAGCGCCATGGAGCCTGACCGCGAATAT
CACTTTGGGCAGGCCGTGAGGTTTGTGTGCAACTCGGGCTACAAAATTGAAGGTGATGAGGAGATGCACTGC
AGCGATGATGGCTTCTGGTCCAAGGAGAAGCCCAAATGTGTGGAGATCTCCTGCAAGTCTCCCGACGTGATC
AACGGCAGCCCAATCAGCCAGAAGATTATTTACAAAGAGAACGAGCGCTTCCAGTACAAGTGTAACATGGGC
TATGAGTATTCAGAGAGGGGAGATGCCGTCTGCACTGAGAGCGGCTGGAGACCACTGCCTAGCTGCGAGGAA
AAGAGTTGTGACAACCCTTACATCCCAAATGGCGACTACTCCCCTCTGCGGATCAAACACCGGACCGGGGAT
GAAATCACCTATCAGTGCCGCAATGGATTCTACCCGGCCACCCGCGGCAACACCGCCAAATGCACCAGCACA
GGCTGGATCCCCGCCCCCCGCTGTACGCTGAAGCCTTGCGACTATCCAGACATCAAGCACGGAGGCCTGTAC
CACGAAAACATGCGGCGGCCTTATTTCCCTGTGGCAGTGGGGAAGTACTACAGCTACTACTGCGACGAGCAC
TTCGAGACCCCCTCTGGCTCCTACTGGGACCACATCCACTGCACACAGGACGGCTGGTCTCCAGCTGTGCCC
TGCCTGAGGAAATGCTACTTCCCCTACCTGGAGAACGGATACAACCAGAACTATGGCCGCAAGTTCGTGCAG
GGCAAGAGCATCGATGTGGCCTGCCACCCTGGCTACGCCCTGCCCAAGGCCCAGACAACTGTGACCTGCATG
GAGAATGGTTGGAGCCCCACCCCGCGCTGCATCCGGGTGTCCTTCACGCTC

In some embodiments, the nucleotide sequence encoding FHL1 has at least 75%, 80%, 85%, 90%, 91%, 92%, 93%, 94%, 95%, 96%, 97%, 98% or 99% identity to SEQ ID NO: 40 or 41, preferably SEQ ID NO: 41. Preferably, the protein encoded by the nucleotide sequence substantially retains a functional activity of the protein represented by SEQ ID NO: 39.

In other embodiments, the nucleotide sequence encoding FHL1 is SEQ ID NO: 40 or 41, preferably SEQ ID NO: 41.

Complement Receptor 1 (CR1)

Complement Receptor 1 (CR1) mediates cellular binding to particles and immune complexes that have activated complement.

In some embodiments, the CR1 is human CR1.

In some embodiments, the amino acid sequence of CR1 is SEQ ID NO: 42. Preferably, a variant or fragment substantially retains a functional activity of the protein represented by SEQ ID NO: 42

(SEQ ID NO: 42)
MGASSPRSPEPVGPPAPGLPFCCGGSLLAVVVLLALPVAWGQCNAPEWLPFARPTNLTDEFEFPIGTYLNYECRP
GYSGRPFSIICLKNSVWTGAKDRCRRKSCRNPPDPVNGMVHVIKGIQFGSQIKYSCTKGYRLIGSSSATCIISGD
TVIWDNETPICDRIPCGLPPTITNGDFISTNRENFHYGSVVTYRCNPGSGGRKVFELVGEPSIYCTSNDDQVGIW
SGPAPQCIIPNKCTPPNVENGILVSDNRSLFSLNEVVEFRCQPGFVMKGPRRVKCQALNKWEPELPSCSRVCQPP
PDVLHAERTQRDKDNFSPGQEVFYSCEPGYDLRGAASMRCTPQGDWSPAAPTCEVKSCDDFMGQLLNGRVLFPVN
LQLGAKVDFVCDEGFQLKGSSASYCVLAGMESLWNSSVPVCEQIFCPSPPVIPNGRHTGKPLEVFPFGKTVNYTC
DPHPDRGTSFDLIGESTIRCTSDPQGNGVWSSPAPRCGILGHCQAPDHFLFAKLKTQTNASDFPIGTSLKYECRP
EYYGRPFSITCLDNLVWSSPKDVCKRKSCKTPPDPVNGMVHVITDIQVGSRINYSCTTGHRLIGHSAECILSGNA
AHWSTKPPICQRIPCGLPPTIANGDFISTNRENFHYGSVVTYRCNPGSGGRKVFELVGEPSIYCTSNDDQVGIWS
GPAPQCIIPNKCTPPNVENGILVSDNRSLFSLNEVVEFRCQPGFVMKGPRRVKCQALNKWEPELPSCSRVCQPPP
DVLHAERTQRDKDNFSPGQEVFYSCEPGYDLRGAASMRCTPQGDWSPAAPTCEVKSCDDFMGQLLNGRVLFPVNL
QLGAKVDFVCDEGFQLKGSSASYCVLAGMESLWNSSVPVCEQIFCPSPPVIPNGRHTGKPLEVFPFGKAVNYTCD
PHPDRGTSFDLIGESTIRCTSDPQGNGVWSSPAPRCGILGHCQAPDHFLFAKLKTQTNASDFPIGTSLKYECRPE
YYGRPFSITCLDNLVWSSPKDVCKRKSCKTPPDPVNGMVHVITDIQVGSRINYSCTTGHRLIGHSSAECILSGNT
AHWSTKPPICQRIPCGLPPTIANGDFISTNRENFHYGSVVTYRCNLGSRGRKVFELVGEPSIYCTSNDDQVGIWS
GPAPQCIIPNKCTPPNVENGILVSDNRSLFSLNEVVEFRCQPGFVMKGPRRVKCQALNKWEPELPSCSRVCQPPP
EILHGEHTPSHQDNFSPGQEVFYSCEPGYDLRGAASLHCTPQGDWSPEAPRCAVKSCDDFLGQLPHGRVLFPLNL
QLGAKVSFVCDEGFRLKGSSVSHCVLVGMRSLWNNSVPVCEHIFCPNPPAILNGRHTGTPSGDIPYGKEISYTCD
PHPDRGMTFNLIGESTIRCTSDPHGNGVWSSPAPRCELSVRAGHCKTPEQFPFASPTIPINDFEFPVGTSLNYEC
RPGYFGKMFSISCLENLVWSSVEDNCRRKSCGPPPEPFNGMVHINTDTQFGSTVNYSCNEGFRLIGSPSTTCLVS
GNNVTWDKKAPICEIISCEPPPTISNGDFYSNNRTSFHNGTVVTYQCHTGPDGEQLFELVGERSIYCTSKDDQVG
VWSSPPPRCISTNKCTAPEVENAIRVPGNRSFFSLTEIIRFRCQPGFVMVGSHTVQCQTNGRWGPKLPHCSRVCQ
PPPEILHGEHTLSHQDNFSPGQEVFYSCEPSYDLRGAASLHCTPQGDWSPEAPRCTVKSCDDFLGQLPHGRVLLP
LNLQLGAKVSFVCDEGFRLKGRSASHCVLAGMKALWNSSVPVCEQIFCPNPPAILNGRHTGTPFGDIPYGKEISY
ACDTHPDRGMTFNLIGESSIRCTSDPQGNGVWSSPAPRCELSVPAACPHPPKIQNGHYIGGHVSLYLPGMTISYI
CDPGYLLVGKGFIFCTDQGIWSQLDHYCKEVNCSFPLFMNGISKELEMKKVYHYGDYVTLKCEDGYTLEGSPWSQ
CQADDRWDPPLAKCTSRTHDALIVGTLSGTIFFILLIIFLSWIILKHRKGNNAHENPKEVAIHLHSQGGSSVHPR
TLQTNEENSRVLP

Membrane Cofactor Protein (MCP)

MCP acts as a cofactor for CFI.

In some embodiments, the MCP is human MCP.

In some embodiments, the amino acid sequence of MCP is SEQ ID NO: 43 or a variant or fragment thereof. Preferably, variant or fragment substantially retains a functional activity of the protein represented by SEQ ID NO: 43

(SEQ ID NO: 43)
MEPPGRRECPFPSWRFPGLLLAAMVLLLYSFSDACEEPPTFEAMELIGKPKPYYEIGERVDYKCKKGYEYIPPLA
THTICDRNHTWLPVSDDACYRETCPYIRDPLNGQAVPANGTYEFGYQMHFICNEGYYLIGEEILYCELKGSVAIW
SGKPPICEKVLCTPPPKIKNGKHTFSEVEVFEYLDAVTYSCDPAPGPDPFSLIGESTIYCGDNSVWSRAAPECKV
VKCRFPVVENGKQISGFGKKFYYKATVMFECDKGFYLDGSDTIVCDSNSTWDPPVPKCLKVLPPSSTKPPALSHS
VSTSSTTKSPASSASGPRPTYKPPVSNYPGYPKPEEGILDSLDVWVIAVIVIAIVVGVAVICVVPYRYLQRRKKK
GTYLTDETHREVKFTSL

Linkers

In preferred embodiments, the nucleotide sequence encoding the anti-VEGF entity is upstream of the nucleotide sequence encoding the negative complement regulator. In other embodiments, the nucleotide sequence encoding the negative complement regulator is upstream of the nucleotide sequence encoding the anti-VEGF entity.

As used herein, “upstream” and “downstream” both refer to relative positions in DNA or RNA. Each strand of DNA or RNA has a 5′ end and a 3′ end and, by convention, “upstream” and “downstream” relate to the 5′ to 3′ direction respectively in which RNA transcription takes place. For example, when considering double-stranded DNA, “upstream” is toward the 5′ end of the coding strand and downstream is toward the 3′ end of the coding strand.

In some embodiments, the nucleotide sequences encoding the anti-VEGF entity and the negative complement regulator are operably linked by a linker. In some embodiments, the linker comprises a self-cleaving 2A peptide sequence, such as a sequence comprising or that is defined by a Furin cleavage site, GSG, 11a1D and an F2A sequence.

In some embodiments, the linker is SEQ ID NO: 44.

(SEQ ID NO: 44)
CGAAGGAAACGAGGAAGCGGAGAAGCCAGACACAAACAGAAAATTGTGG
CACCGGTGAAACAGACTTTGAATTTTGACCTTCTCAAGTTGGCGGGAGA
CGTCGAGTCCAACCCTGGGCCC

In other embodiments, the linker has at least 75%, 80%, 85%, 86%, 87%, 88%, 89%, 90%, 91%, 92%, 93%, 94%, 95%, 96%, 97%, 98% or 99% identity to SEQ ID NO: 44. Preferably, the linker substantially retains a functional activity of SEQ ID NO: 44.

By “operably linked”, it is to be understood that the individual components are linked together in a manner which enables them to carry out their function substantially unhindered.

Product

The product of the invention may, for example, be a composition (e.g. a pharmaceutical composition) comprising (i) an anti-VEGF entity; and (ii) a negative complement regulator, or nucleotide sequences encoding therefor, in admixture. Alternatively, the product may, for example, be a kit comprising preparations of (i) an anti-VEGF entity; and (ii) a negative complement regulator, or nucleotide sequences encoding therefor, and, optionally, instructions for the simultaneous, sequential or separate administration of the preparations to a subject in need thereof.

Protein Transduction

As an alternative to the delivery of polynucleotides to cells, the products and agents of the invention may be delivered to cells by protein transduction.

Protein transduction may be via vector delivery (Cai, Y. et al. (2014) Elife 3: e01911; Maetzig, T. et al. (2012) Curr. Gene Ther. 12: 389-409). Vector delivery involves the engineering of viral particles (e.g. lentiviral particles) to comprise the proteins to be delivered to a cell. Accordingly, when the engineered viral particles enter a cell as part of their natural life cycle, the proteins comprised in the particles are carried into the cell.

Protein transduction may be via protein delivery (Gaj, T. et al. (2012) Nat. Methods 9: 805-7). Protein delivery may be achieved, for example, by utilising a vehicle (e.g. liposomes) or even by administering the protein itself directly to a cell.

Polynucleotide

Polynucleotides of the invention may comprise DNA or RNA, preferably DNA. They may be single-stranded or double-stranded. It will be understood by a skilled person that numerous different polynucleotides can encode the same polypeptide as a result of the degeneracy of the genetic code. In addition, it is to be understood that skilled persons may, using routine techniques, make nucleotide substitutions that do not affect the polypeptide sequence encoded by the polynucleotides of the invention to reflect the codon usage of any particular host organism in which the polypeptides of the invention are to be expressed.

The nucleotide sequences of the invention disclosed herein may comprise or lack stop codons at their 3′ end, for example depending on their position in a bicistronic vector. Thus, the present disclosure encompasses the SEQ ID NOs disclosed herein with the stop codons present or absent.

The polynucleotides may be modified by any method available in the art. Such modifications may be carried out in order to enhance the in vivo activity or lifespan of the polynucleotides of the invention.

Polynucleotides such as DNA polynucleotides may be produced recombinantly, synthetically or by any means available to those of skill in the art. They may also be cloned by standard techniques.

Longer polynucleotides will generally be produced using recombinant means, for example using polymerase chain reaction (PCR) cloning techniques. This will involve making a pair of primers (e.g. of about 15 to 30 nucleotides) flanking the target sequence which it is desired to clone, bringing the primers into contact with mRNA or cDNA obtained from an animal or human cell, performing a polymerase chain reaction under conditions which bring about amplification of the desired region, isolating the amplified fragment (e.g. by purifying the reaction mixture with an agarose gel) and recovering the amplified DNA. The primers may be designed to contain suitable restriction enzyme recognition sites so that the amplified DNA can be cloned into a suitable vector.

Polynucleotides may be delivered to cells by plasmid transfection or electroporation; for example as non-viral vectors such as naked DNA plasmids, bacterial artificial chromosomes, mini-circle technologies. These entities can also package larger transgenes than AAV vectors but may at a slight disadvantage of being less stable, bioavailability and target specificity. For example, Eyevensys uses a naked DNA electroporation platform for delivering therapeutic transgenes to muscles/eye etc.

In some embodiments, the polynucleotide comprises or consists of the nucleotide sequence of SEQ ID NO: 45, or a nucleotide sequence that has at least 75%, 80%, 85%, 86%, 87%, 88%, 89%, 90%, 91%, 92%, 93%, 94%, 95%, 96%, 97%, 98% or 99% sequence identity thereto.

(SEQ ID NO: 45)
CGCTCGCTCACTGAGGCCGCCCGGGCAAAGCCCGGGCGTCGGGCGACCTTTGGTCGCCCGGCCTCAGTGAGCGAG
CGAGCGCGCAGAGAGGGAGTGGCCAACTCCATCACTAGGGGTTCCTGGCGCGCCGGAGTTCCGCGTTACATAACT
TACGGTAAATGGCCCGCCTGGCTGACCGCCCAACGACCCCCGCCCATTGACGTCAATAATGACGTATGTTCCCAT
AGTAACGCCAATAGGGACTTTCCATTGACGTCAATGGGTGGAGTATTTACGGTAAACTGCCCACTTGGCAGTACA
TCAAGTGTATCATATGCCAAGTACGCCCCCTATTGACGTCAATGACGGTAAATGGCCCGCCTGGCATTATGCCCA
GTACATGACCTTATGGGACTTTCCTACTTGGCAGTACATCTACGTATTAGTCATCGCTATTACCATGGTGATGCG
GTTTTGGCAGTACATCAATGGGCGTGGATAGCGGTTTGACTCACGGGGATTTCCAAGTCTCCACCCCATTGACGT
CAATGGGAGTTTGTTTTGGCACCAAAATCAACGGGACTTTCCAAAATGTCGTAACAACTCCGCCCCATTGACGCA
AATGGGCGGTAGGCGTGTACGGTGGGAGGTCTATATAAGCAGAGCTCGTTTAGTGAACCGTCAGATCGCCTGGAG
ACGCCATCCACGCTGTTTTGACCTCCATAGAAGACACCGACTAGTGCCACCATGAAACTGCTGCATGTCTTCCTC
CTCTTCCTGTGCTTCCACCTCCGTTTCTGTAAAGTCACCTACACTAGCCAGGAGGATCTGGTGGAGAAGAAATGC
CTGGCCAAGAAGTATACCCACCTGAGCTGCGACAAAGTGTTCTGCCAGCCCTGGCAACGCTGCATTGAAGGTACT
TGTGTGTGCAAGCTGCCCTACCAGTGCCCCAAGAACGGCACGGCCGTGTGTGCCACCAACAGGAGGAGCTTCCCC
ACCTACTGCCAGCAGAAGAGCCTGGAATGCCTCCACCCTGGCACCAAGTTTCTGAACAACGGGACCTGCACAGCC
GAGGGGAAATTCAGCGTCTCCCTCAAGCACGGCAATACAGACTCCGAGGGCATTGTGGAAGTGAAGCTGGTGGAC
CAGGACAAGACCATGTTCATCTGCAAAAGCAGCTGGTCCATGCGGGAGGCCAATGTCGCCTGCCTGGACCTGGGC
TTCCAGCAGGGCGCTGATACACAGCGCCGCTTTAAACTCAGTGACCTCAGCATCAACAGCACTGAGTGTCTGCAC
GTGCACTGCCGGGGCCTGGAGACCAGCCTGGCTGAGTGCACCTTCACCAAGCGCAGGACCATGGGCTACCAGGAT
TTTGCAGATGTGGTCTGCTACACCCAGAAGGCAGACAGCCCCATGGATGACTTCTTCCAGTGTGTCAATGGCAAG
TACATTTCCCAGATGAAGGCTTGTGACGGGATCAATGATTGCGGGGATCAGAGCGATGAGCTCTGCTGCAAGGCC
TGCCAAGGGAAGGGCTTTCACTGTAAGTCTGGGGTGTGCATCCCTTCTCAGTATCAGTGCAACGGAGAGGTGGAC
TGCATCACTGGGGAGGACGAGGTGGGCTGTGCTGGCTTCGCCTCTGTGGCCCAGGAGGAGACAGAGATCCTCACA
GCTGACATGGATGCAGAGCGGCGGCGCATCAAGAGTCTGCTCCCAAAGCTCTCCTGCGGCGTTAAGAATCGCATG
CACATCCGGAGGAAGCGGATCGTTGGAGGCAAACGGGCTCAGCTGGGGGACTTGCCGTGGCAGGTGGCCATCAAA
GATGCCTCCGGAATCACCTGTGGTGGCATCTACATCGGCGGCTGCTGGATCCTGACCGCCGCCCACTGCCTTCGG
GCCAGCAAGACTCACCGCTACCAGATCTGGACCACCGTGGTGGATTGGATTCACCCCGACCTGAAGAGGATTGTC
ATTGAGTATGTCGACCGCATCATCTTCCATGAAAACTACAATGCCGGGACGTATCAGAACGACATCGCCCTCATC
GAGATGAAGAAGGATGGGAACAAGAAGGACTGTGAGCTGCCTCGCTCCATCCCCGCCTGTGTACCATGGTCTCCG
TACCTGTTCCAGCCAAATGACACATGCATCGTGAGCGGCTGGGGCCGCGAGAAAGACAACGAGAGGGTCTTCTCC
CTGCAGTGGGGTGAAGTCAAGCTGATCAGCAACTGCTCCAAGTTCTACGGCAACCGCTTCTATGAGAAGGAGATG
GAGTGCGCCGGCACCTATGACGGCAGCATTGACGCGTGCAAGGGAGACAGTGGGGGCCCCCTGGTCTGCATGGAC
GCCAACAATGTGACCTACGTGTGGGGAGTTGTGTCCTGGGGCGAGAACTGTGGCAAGCCTGAGTTCCCGGGCGTG
TACACAAAGGTGGCAAACTATTTTGACTGGATCTCCTATCACGTTGGCAGGCCCTTCATTTCACAGTACAACGTA
CGAAGGAAACGAGGAAGCGGAGAAGCCAGACACAAACAGAAAATTGTGGCACCGGTGAAACAGACTTTGAATTTT
GACCTTCTCAAGTTGGCGGGAGACGTCGAGTCCAACCCTGGGCCCATGCGCCTGCTGGCCAAGATCATCTGCCTG
ATGCTGTGGGCCATCTGCGTGGCCAGCGACACCGGCCGCCCCTTCGTGGAGATGTACAGCGAGATCCCCGAGATC
ATCCACATGACCGAGGGCCGCGAGCTGGTGATCCCCTGCCGCGTGACCAGCCCCAACATCACCGTGACCCTGAAG
AAGTTCCCCCTGGACACCCTGATCCCCGACGGCAAGCGCATCATCTGGGACAGCCGCAAGGGCTTCATCATCAGC
AACGCCACCTACAAGGAGATCGGCCTGCTGACCTGCGAGGCCACCGTGAACGGCCACCTGTACAAGACCAACTAC
CTGACCCACCGCCAGACCAACACCATCATCGACGTGGTGCTGAGCCCCAGCCACGGCATCGAGCTGAGCGTGGGC
GAGAAGCTGGTGCTGAACTGCACCGCCCGCACCGAGCTGAACGTGGGCATCGACTTCAACTGGGAGTACCCCAGC
AGCAAGCACCAGCACAAGAAGCTGGTGAACCGCGACCTGAAGACCCAGAGCGGCAGCGAGATGAAGAAGTTCCTG
AGCACCCTGACCATCGACGGCGTGACCCGCAGCGACCAGGGCCTGTACACCTGCGCCGCCAGCAGCGGCCTGATG
ACCAAGAAGAACAGCACCTTCGTGCGCGTGCACGAGAAGGACAAGACCCACACCTGCCCCCCCTGCCCCGCCCCC
GAGCTGCTGGGCGGCCCCAGCGTGTTCCTGTTCCCCCCCAAGCCCAAGGACACCCTGATGATCAGCCGCACCCCC
GAGGTGACCTGCGTGGTGGTGGACGTGAGCCACGAGGACCCCGAGGTGAAGTTCAACTGGTACGTGGACGGCGTG
GAGGTGCACAACGCCAAGACCAAGCCCCGCGAGGAGCAGTACAACAGCACCTACCGCGTGGTGAGCGTGCTGACC
GTGCTGCACCAGGACTGGCTGAACGGCAAGGAGTACAAGTGCAAGGTGAGCAACAAGGCCCTGCCCGCCCCCATC
GAGAAGACCATCAGCAAGGCCAAGGGCCAGCCCCGCGAGCCCCAGGTGTACACCCTGCCCCCCAGCCGCGACGAG
CTGACCAAGAACCAGGTGAGCCTGACCTGCCTGGTGAAGGGCTTCTACCCCAGCGACATCGCCGTGGAGTGGGAG
AGCAACGGCCAGCCCGAGAACAACTACAAGACCACCCCCCCCGTGCTGGACAGCGACGGCAGCTTCTTCCTGTAC
AGCAAGCTGACCGTGGACAAGAGCCGCTGGCAGCAGGGCAACGTGTTCAGCTGCAGCGTGATGCACGAGGCCCTG
CACAACCACTACACCCAGAAGAGCCTGAGCCTGAGCCCCGGCTAACTCGAGAATCAACCTCTGGATTACAAAATT
TGTGAAAGATTGACTGGTATTCTTAACTATGTTGCTCCTTTTACGCTATGTGGATACGCTGCTTTAATGCCTTTG
TATCATGCTATTGCTTCCCGTATGGCTTTCATTTTCTCCTCCTTGTATAAATCCTGGTTAGTTCTTGCCACGGCG
GAACTCATCGCCGCCTGCCTTGCCCGCTGCTGGACAGGGGCTCGGCTGTTGGGCACTGACAATTCCGTGGTGTGC
CTTCTAGTTGCCAGCCATCTGTTGTTTGCCCCTCCCCCGTGCCTTCCTTGACCCTGGAAGGTGCCACTCCCACTG
TCCTTTCCTAATAAAATGAGGAAATTGCATCGCATTGTCTGAGTAGGTGTCATTCTATTCTGGGGGGTGGGGTGG
GGCAGGACAGCAAGGGGGAGGATTGGGAAGACAATAGCAGGCATGCTGGGGATGCGGTGGGCTCTATGGGCGGCC
GCAGGAACCCCTAGTGATGGAGTTGGCCACTCCCTCTCTGCGCGCTCGCTCGCTCACTGAGGCCGGGCGACCAAA
GGTCGCCCGACGCCCGGGCTTTGCCCGGGCGGCCTCAGTGAGCGAGCG

In some embodiments, the polynucleotide comprises or consists of the nucleotide sequence of SEQ ID NO: 46, or a nucleotide sequence that has at least 75%, 80%, 85%, 86%, 87%, 88%, 89%, 90%, 91%, 92%, 93%, 94%, 95%, 96%, 97%, 98% or 99% sequence identity thereto.

(SEQ ID NO: 46)
CGCTCGCTCACTGAGGCCGCCCGGGCAAAGCCCGGGCGTCGGGCGACCTTTGGTCGCCCGGCCTCAGTGAGC
GAGCGAGCGCGCAGAGAGGGAGTGGCCAACTCCATCACTAGGGGTTCCTGGCGCGCCGGAGTTCCGCGTTAC
ATAACTTACGGTAAATGGCCCGCCTGGCTGACCGCCCAACGACCCCCGCCCATTGACGTCAATAATGACGTA
TGTTCCCATAGTAACGCCAATAGGGACTTTCCATTGACGTCAATGGGTGGAGTATTTACGGTAAACTGCCCA
CTTGGCAGTACATCAAGTGTATCATATGCCAAGTACGCCCCCTATTGACGTCAATGACGGTAAATGGCCCGC
CTGGCATTATGCCCAGTACATGACCTTATGGGACTTTCCTACTTGGCAGTACATCTACGTATTAGTCATCGC
TATTACCATGGTGATGCGGTTTTGGCAGTACATCAATGGGCGTGGATAGCGGTTTGACTCACGGGGATTTCC
AAGTCTCCACCCCATTGACGTCAATGGGAGTTTGTTTTGGCACCAAAATCAACGGGACTTTCCAAAATGTCG
TAACAACTCCGCCCCATTGACGCAAATGGGCGGTAGGCGTGTACGGTGGGAGGTCTATATAAGCAGAGCTCG
TTTAGTGAACCGTCAGATCGCCTGGAGACGCCATCCACGCTGTTTTGACCTCCATAGAAGACACCGACTAGT
GCCACCATGCGCCTGCTGGCCAAGATCATCTGCCTGATGCTGTGGGCCATCTGCGTGGCCAGCGACACCGGC
CGCCCCTTCGTGGAGATGTACAGCGAGATCCCCGAGATCATCCACATGACCGAGGGCCGCGAGCTGGTGATC
CCCTGCCGCGTGACCAGCCCCAACATCACCGTGACCCTGAAGAAGTTCCCCCTGGACACCCTGATCCCCGAC
GGCAAGCGCATCATCTGGGACAGCCGCAAGGGCTTCATCATCAGCAACGCCACCTACAAGGAGATCGGCCTG
CTGACCTGCGAGGCCACCGTGAACGGCCACCTGTACAAGACCAACTACCTGACCCACCGCCAGACCAACACC
ATCATCGACGTGGTGCTGAGCCCCAGCCACGGCATCGAGCTGAGCGTGGGCGAGAAGCTGGTGCTGAACTGC
ACCGCCCGCACCGAGCTGAACGTGGGCATCGACTTCAACTGGGAGTACCCCAGCAGCAAGCACCAGCACAAG
AAGCTGGTGAACCGCGACCTGAAGACCCAGAGCGGCAGCGAGATGAAGAAGTTCCTGAGCACCCTGACCATC
GACGGCGTGACCCGCAGCGACCAGGGCCTGTACACCTGCGCCGCCAGCAGCGGCCTGATGACCAAGAAGAAC
AGCACCTTCGTGCGCGTGCACGAGAAGGACAAGACCCACACCTGCCCCCCCTGCCCCGCCCCCGAGCTGCTG
GGCGGCCCCAGCGTGTTCCTGTTCCCCCCCAAGCCCAAGGACACCCTGATGATCAGCCGCACCCCCGAGGTG
ACCTGCGTGGTGGTGGACGTGAGCCACGAGGACCCCGAGGTGAAGTTCAACTGGTACGTGGACGGCGTGGAG
GTGCACAACGCCAAGACCAAGCCCCGCGAGGAGCAGTACAACAGCACCTACCGCGTGGTGAGCGTGCTGACC
GTGCTGCACCAGGACTGGCTGAACGGCAAGGAGTACAAGTGCAAGGTGAGCAACAAGGCCCTGCCCGCCCCC
ATCGAGAAGACCATCAGCAAGGCCAAGGGCCAGCCCCGCGAGCCCCAGGTGTACACCCTGCCCCCCAGCCGC
GACGAGCTGACCAAGAACCAGGTGAGCCTGACCTGCCTGGTGAAGGGCTTCTACCCCAGCGACATCGCCGTG
GAGTGGGAGAGCAACGGCCAGCCCGAGAACAACTACAAGACCACCCCCCCCGTGCTGGACAGCGACGGCAGC
TTCTTCCTGTACAGCAAGCTGACCGTGGACAAGAGCCGCTGGCAGCAGGGCAACGTGTTCAGCTGCAGCGTG
ATGCACGAGGCCCTGCACAACCACTACACCCAGAAGAGCCTGAGCCTGAGCCCCGGCTAACGAAGGAAACGA
GGAAGCGGAGAAGCCAGACACAAACAGAAAATTGTGGCACCGGTGAAACAGACTTTGAATTTTGACCTTCTC
AAGTTGGCGGGAGACGTCGAGTCCAACCCTGGGCCCATGAAACTGCTGCATGTCTTCCTCCTCTTCCTGTGC
TTCCACCTCCGTTTCTGTAAAGTCACCTACACTAGCCAGGAGGATCTGGTGGAGAAGAAATGCCTGGCCAAG
AAGTATACCCACCTGAGCTGCGACAAAGTGTTCTGCCAGCCCTGGCAACGCTGCATTGAAGGTACTTGTGTG
TGCAAGCTGCCCTACCAGTGCCCCAAGAACGGCACGGCCGTGTGTGCCACCAACAGGAGGAGCTTCCCCACC
TACTGCCAGCAGAAGAGCCTGGAATGCCTCCACCCTGGCACCAAGTTTCTGAACAACGGGACCTGCACAGCC
GAGGGGAAATTCAGCGTCTCCCTCAAGCACGGCAATACAGACTCCGAGGGCATTGTGGAAGTGAAGCTGGTG
GACCAGGACAAGACCATGTTCATCTGCAAAAGCAGCTGGTCCATGCGGGAGGCCAATGTCGCCTGCCTGGAC
CTGGGCTTCCAGCAGGGCGCTGATACACAGCGCCGCTTTAAACTCAGTGACCTCAGCATCAACAGCACTGAG
TGTCTGCACGTGCACTGCCGGGGCCTGGAGACCAGCCTGGCTGAGTGCACCTTCACCAAGCGCAGGACCATG
GGCTACCAGGATTTTGCAGATGTGGTCTGCTACACCCAGAAGGCAGACAGCCCCATGGATGACTTCTTCCAG
TGTGTCAATGGCAAGTACATTTCCCAGATGAAGGCTTGTGACGGGATCAATGATTGCGGGGATCAGAGCGAT
GAGCTCTGCTGCAAGGCCTGCCAAGGGAAGGGCTTTCACTGTAAGTCTGGGGTGTGCATCCCTTCTCAGTAT
CAGTGCAACGGAGAGGTGGACTGCATCACTGGGGAGGACGAGGTGGGCTGTGCTGGCTTCGCCTCTGTGGCC
CAGGAGGAGACAGAGATCCTCACAGCTGACATGGATGCAGAGCGGCGGCGCATCAAGAGTCTGCTCCCAAAG
CTCTCCTGCGGCGTTAAGAATCGCATGCACATCCGGAGGAAGCGGATCGTTGGAGGCAAACGGGCTCAGCTG
GGGGACTTGCCGTGGCAGGTGGCCATCAAAGATGCCTCCGGAATCACCTGTGGTGGCATCTACATCGGCGGC
TGCTGGATCCTGACCGCCGCCCACTGCCTTCGGGCCAGCAAGACTCACCGCTACCAGATCTGGACCACCGTG
GTGGATTGGATTCACCCCGACCTGAAGAGGATTGTCATTGAGTATGTCGACCGCATCATCTTCCATGAAAAC
TACAATGCCGGGACGTATCAGAACGACATCGCCCTCATCGAGATGAAGAAGGATGGGAACAAGAAGGACTGT
GAGCTGCCTCGCTCCATCCCCGCCTGTGTACCATGGTCTCCGTACCTGTTCCAGCCAAATGACACATGCATC
GTGAGCGGCTGGGGCCGCGAGAAAGACAACGAGAGGGTCTTCTCCCTGCAGTGGGGTGAAGTCAAGCTGATC
AGCAACTGCTCCAAGTTCTACGGCAACCGCTTCTATGAGAAGGAGATGGAGTGCGCCGGCACCTATGACGGC
AGCATTGACGCGTGCAAGGGAGACAGTGGGGGCCCCCTGGTCTGCATGGACGCCAACAATGTGACCTACGTG
TGGGGAGTTGTGTCCTGGGGCGAGAACTGTGGCAAGCCTGAGTTCCCGGGCGTGTACACAAAGGTGGCAAAC
TATTTTGACTGGATCTCCTATCACGTTGGCAGGCCCTTCATTTCACAGTACAACGTACTCGAGAATCAACCT
CTGGATTACAAAATTTGTGAAAGATTGACTGGTATTCTTAACTATGTTGCTCCTTTTACGCTATGTGGATAC
GCTGCTTTAATGCCTTTGTATCATGCTATTGCTTCCCGTATGGCTTTCATTTTCTCCTCCTTGTATAAATCC
TGGTTAGTTCTTGCCACGGCGGAACTCATCGCCGCCTGCCTTGCCCGCTGCTGGACAGGGGCTCGGCTGTTG
GGCACTGACAATTCCGTGGTGTGCCTTCTAGTTGCCAGCCATCTGTTGTTTGCCCCTCCCCCGTGCCTTCCT
TGACCCTGGAAGGTGCCACTCCCACTGTCCTTTCCTAATAAAATGAGGAAATTGCATCGCATTGTCTGAGTA
GGTGTCATTCTATTCTGGGGGGTGGGGTGGGGCAGGACAGCAAGGGGGAGGATTGGGAAGACAATAGCAGGC
ATGCTGGGGATGCGGTGGGCTCTATGGGCGGCCGCAGGAACCCCTAGTGATGGAGTTGGCCACTCCCTCTCT
GCGCGCTCGCTCGCTCACTGAGGCCGGGCGACCAAAGGTCGCCCGACGCCCGGGCTTTGCCCGGGCGGCCTC
AGTGAGCGAGCG

In some embodiments, the polynucleotide comprises or consists of the nucleotide sequence of SEQ ID NO: 47, or a nucleotide sequence that has at least 75%, 80%, 85%, 86%, 87%, 88%, 89%, 90%, 91%, 92%, 93%, 94%, 95%, 96%, 97%, 98% or 99% sequence identity thereto.

(SEQ ID NO: 47)
CGCTCGCTCACTGAGGCCGCCCGGGCAAAGCCCGGGCGTCGGGCGACCTTTGGTCGCCCGGCCTCAGTGAGCGAG
CGAGCGCGCAGAGAGGGAGTGGCCAACTCCATCACTAGGGGTTCCTGGCGCGCCGGAGTTCCGCGTTACATAACT
TACGGTAAATGGCCCGCCTGGCTGACCGCCCAACGACCCCCGCCCATTGACGTCAATAATGACGTATGTTCCCAT
AGTAACGCCAATAGGGACTTTCCATTGACGTCAATGGGTGGAGTATTTACGGTAAACTGCCCACTTGGCAGTACA
TCAAGTGTATCATATGCCAAGTACGCCCCCTATTGACGTCAATGACGGTAAATGGCCCGCCTGGCATTATGCCCA
GTACATGACCTTATGGGACTTTCCTACTTGGCAGTACATCTACGTATTAGTCATCGCTATTACCATGGTGATGCG
GTTTTGGCAGTACATCAATGGGCGTGGATAGCGGTTTGACTCACGGGGATTTCCAAGTCTCCACCCCATTGACGT
CAATGGGAGTTTGTTTTGGCACCAAAATCAACGGGACTTTCCAAAATGTCGTAACAACTCCGCCCCATTGACGCA
AATGGGCGGTAGGCGTGTACGGTGGGAGGTCTATATAAGCAGAGCTCGTTTAGTGAACCGTCAGATCGCCTGGAG
ACGCCATCCACGCTGTTTTGACCTCCATAGAAGACACCGACTAGTGCCACCATGAAACTGCTGCATGTCTTCCTC
CTCTTCCTGTGCTTCCACCTCCGTTTCTGTAAAGTCACCTACACTAGCCAGGAGGATCTGGTGGAGAAGAAATGC
CTGGCCAAGAAGTATACCCACCTGAGCTGCGACAAAGTGTTCTGCCAGCCCTGGCAACGCTGCATTGAAGGTACT
TGTGTGTGCAAGCTGCCCTACCAGTGCCCCAAGAACGGCACGGCCGTGTGTGCCACCAACAGGAGGAGCTTCCCC
ACCTACTGCCAGCAGAAGAGCCTGGAATGCCTCCACCCTGGCACCAAGTTTCTGAACAACGGGACCTGCACAGCC
GAGGGGAAATTCAGCGTCTCCCTCAAGCACGGCAATACAGACTCCGAGGGCATTGTGGAAGTGAAGCTGGTGGAC
CAGGACAAGACCATGTTCATCTGCAAAAGCAGCTGGTCCATGCGGGAGGCCAATGTCGCCTGCCTGGACCTGGGC
TTCCAGCAGGGCGCTGATACACAGCGCCGCTTTAAACTCAGTGACCTCAGCATCAACAGCACTGAGTGTCTGCAC
GTGCACTGCCGGGGCCTGGAGACCAGCCTGGCTGAGTGCACCTTCACCAAGCGCAGGACCATGGGCTACCAGGAT
TTTGCAGATGTGGTCTGCTACACCCAGAAGGCAGACAGCCCCATGGATGACTTCTTCCAGTGTGTCAATGGCAAG
TACATTTCCCAGATGAAGGCTTGTGACGGGATCAATGATTGCGGGGATCAGAGCGATGAGCTCTGCTGCAAGGCC
TGCCAAGGGAAGGGCTTTCACTGTAAGTCTGGGGTGTGCATCCCTTCTCAGTATCAGTGCAACGGAGAGGTGGAC
TGCATCACTGGGGAGGACGAGGTGGGCTGTGCTGGCTTCGCCTCTGTGGCCCAGGAGGAGACAGAGATCCTCACA
GCTGACATGGATGCAGAGCGGCGGCGCATCAAGAGTCTGCTCCCAAAGCTCTCCTGCGGCGTTAAGAATCGCATG
CACATCCGGAGGAAGCGGATCGTTGGAGGCAAACGGGCTCAGCTGGGGGACTTGCCGTGGCAGGTGGCCATCAAA
GATGCCTCCGGAATCACCTGTGGTGGCATCTACATCGGCGGCTGCTGGATCCTGACCGCCGCCCACTGCCTTCGG
GCCAGCAAGACTCACCGCTACCAGATCTGGACCACCGTGGTGGATTGGATTCACCCCGACCTGAAGAGGATTGTC
ATTGAGTATGTCGACCGCATCATCTTCCATGAAAACTACAATGCCGGGACGTATCAGAACGACATCGCCCTCATC
GAGATGAAGAAGGATGGGAACAAGAAGGACTGTGAGCTGCCTCGCTCCATCCCCGCCTGTGTACCATGGTCTCCG
TACCTGTTCCAGCCAAATGACACATGCATCGTGAGCGGCTGGGGCCGCGAGAAAGACAACGAGAGGGTCTTCTCC
CTGCAGTGGGGTGAAGTCAAGCTGATCAGCAACTGCTCCAAGTTCTACGGCAACCGCTTCTATGAGAAGGAGATG
GAGTGCGCCGGCACCTATGACGGCAGCATTGACGCGTGCAAGGGAGACAGTGGGGGCCCCCTGGTCTGCATGGAC
GCCAACAATGTGACCTACGTGTGGGGAGTTGTGTCCTGGGGCGAGAACTGTGGCAAGCCTGAGTTCCCGGGCGTG
TACACAAAGGTGGCAAACTATTTTGACTGGATCTCCTATCACGTTGGCAGGCCCTTCATTTCACAGTACAACGTA
CGAAGGAAACGAGGAAGCGGAGAAGCCAGACACAAACAGAAAATTGTGGCACCGGTGAAACAGACTTTGAATTTT
GACCTTCTCAAGTTGGCGGGAGACGTCGAGTCCAACCCTGGGCCCATGCGCCTGCTGGCCAAGATCATCTGCCTG
ATGCTGTGGGCCATCTGCGTGGCCAGCGACACCGGCCGCCCCTTCGTGGAGATGTACAGCGAGATCCCCGAGATC
ATCCACATGACCGAGGGCCGCGAGCTGGTGATCCCCTGCCGCGTGACCAGCCCCAACATCACCGTGACCCTGAAG
AAGTTCCCCCTGGACACCCTGATCCCCGACGGCAAGCGCATCATCTGGGACAGCCGCAAGGGCTTCATCATCAGC
AACGCCACCTACAAGGAGATCGGCCTGCTGACCTGCGAGGCCACCGTGAACGGCCACCTGTACAAGACCAACTAC
CTGACCCACCGCCAGACCAACACCATCATCGACGTGGTGCTGAGCCCCAGCCACGGCATCGAGCTGAGCGTGGGC
GAGAAGCTGGTGCTGAACTGCACCGCCCGCACCGAGCTGAACGTGGGCATCGACTTCAACTGGGAGTACCCCAGC
AGCAAGCACCAGCACAAGAAGCTGGTGAACCGCGACCTGAAGACCCAGAGCGGCAGCGAGATGAAGAAGTTCCTG
AGCACCCTGACCATCGACGGCGTGACCCGCAGCGACCAGGGCCTGTACACCTGCGCCGCCAGCAGCGGCCTGATG
ACCAAGAAGAACAGCACCTTCGTGCGCGTGCACGAGAAGGACAAGACCCACACCTGCCCCCCCTGCCCCGCCCCC
GAGCTGCTGGGCGGCCCCAGCGTGTTCCTGTTCCCCCCCAAGCCCAAGGACACCCTGATGATCAGCCGCACCCCC
GAGGTGACCTGCGTGGTGGTGGACGTGAGCCACGAGGACCCCGAGGTGAAGTTCAACTGGTACGTGGACGGCGTG
GAGGTGCACAACGCCAAGACCAAGCCCCGCGAGGAGCAGTACAACAGCACCTACCGCGTGGTGAGCGTGCTGACC
GTGCTGCACCAGGACTGGCTGAACGGCAAGGAGTACAAGTGCAAGGTGAGCAACAAGGCCCTGCCCGCCCCCATC
GAGAAGACCATCAGCAAGGCCAAGGGCCAGCCCCGCGAGCCCCAGGTGTACACCCTGCCCCCCAGCCGCGACGAG
CTGACCAAGAACCAGGTGAGCCTGACCTGCCTGGTGAAGGGCTTCTACCCCAGCGACATCGCCGTGGAGTGGGAG
AGCAACGGCCAGCCCGAGAACAACTACAAGACCACCCCCCCCGTGCTGGACAGCGACGGCAGCTTCTTCCTGTAC
AGCAAGCTGACCGTGGACAAGAGCCGCTGGCAGCAGGGCAACGTGTTCAGCTGCAGCGTGATGCACGAGGCCCTG
CACAACCACTACACCCAGAAGAGCCTGAGCCTGAGCCCCGGCTAACTCGAGGTGCCTTCTAGTTGCCAGCCATCT
GTTGTTTGCCCCTCCCCCGTGCCTTCCTTGACCCTGGAAGGTGCCACTCCCACTGTCCTTTCCTAATAAAATGAG
GAAATTGCATCGCATTGTCTGAGTAGGTGTCATTCTATTCTGGGGGGTGGGGTGGGGCAGGACAGCAAGGGGGAG
GATTGGGAAGACAATAGCAGGCATGCTGGGGATGCGGTGGGCTCTATGGGCGGCCGCAGGAACCCCTAGTGATGG
AGTTGGCCACTCCCTCTCTGCGCGCTCGCTCGCTCACTGAGGCCGGGCGACCAAAGGTCGCCCGACGCCCGGGCT
TTGCCCGGGCGGCCTCAGTGAGCGAGCG

In some embodiments, the polynucleotide comprises or consists of the nucleotide sequence of SEQ ID NO: 48, or a nucleotide sequence that has at least 75%, 80%, 85%, 86%, 87%, 88%, 89%, 90%, 91%, 92%, 93%, 94%, 95%, 96%, 97%, 98% or 99% sequence identity thereto.

(SEQ ID NO: 48)
CGCTCGCTCACTGAGGCCGCCCGGGCAAAGCCCGGGCGTCGGGCGACCTTTGGTCGCCCGGCCTCAGTGAGC
GAGCGAGCGCGCAGAGAGGGAGTGGCCAACTCCATCACTAGGGGTTCCTGGCGCGCCGGAGTTCCGCGTTAC
ATAACTTACGGTAAATGGCCCGCCTGGCTGACCGCCCAACGACCCCCGCCCATTGACGTCAATAATGACGTA
TGTTCCCATAGTAACGCCAATAGGGACTTTCCATTGACGTCAATGGGTGGAGTATTTACGGTAAACTGCCCA
CTTGGCAGTACATCAAGTGTATCATATGCCAAGTACGCCCCCTATTGACGTCAATGACGGTAAATGGCCCGC
CTGGCATTATGCCCAGTACATGACCTTATGGGACTTTCCTACTTGGCAGTACATCTACGTATTAGTCATCGC
TATTACCATGGTGATGCGGTTTTGGCAGTACATCAATGGGCGTGGATAGCGGTTTGACTCACGGGGATTTCC
AAGTCTCCACCCCATTGACGTCAATGGGAGTTTGTTTTGGCACCAAAATCAACGGGACTTTCCAAAATGTCG
TAACAACTCCGCCCCATTGACGCAAATGGGCGGTAGGCGTGTACGGTGGGAGGTCTATATAAGCAGAGCTCG
TTTAGTGAACCGTCAGATCGCCTGGAGACGCCATCCACGCTGTTTTGACCTCCATAGAAGACACCGACTAGT
GCCACCATGCGCCTGCTGGCCAAGATCATCTGCCTGATGCTGTGGGCCATCTGCGTGGCCAGCGACACCGGC
CGCCCCTTCGTGGAGATGTACAGCGAGATCCCCGAGATCATCCACATGACCGAGGGCCGCGAGCTGGTGATC
CCCTGCCGCGTGACCAGCCCCAACATCACCGTGACCCTGAAGAAGTTCCCCCTGGACACCCTGATCCCCGAC
GGCAAGCGCATCATCTGGGACAGCCGCAAGGGCTTCATCATCAGCAACGCCACCTACAAGGAGATCGGCCTG
CTGACCTGCGAGGCCACCGTGAACGGCCACCTGTACAAGACCAACTACCTGACCCACCGCCAGACCAACACC
ATCATCGACGTGGTGCTGAGCCCCAGCCACGGCATCGAGCTGAGCGTGGGCGAGAAGCTGGTGCTGAACTGC
ACCGCCCGCACCGAGCTGAACGTGGGCATCGACTTCAACTGGGAGTACCCCAGCAGCAAGCACCAGCACAAG
AAGCTGGTGAACCGCGACCTGAAGACCCAGAGCGGCAGCGAGATGAAGAAGTTCCTGAGCACCCTGACCATC
GACGGCGTGACCCGCAGCGACCAGGGCCTGTACACCTGCGCCGCCAGCAGCGGCCTGATGACCAAGAAGAAC
AGCACCTTCGTGCGCGTGCACGAGAAGGACAAGACCCACACCTGCCCCCCCTGCCCCGCCCCCGAGCTGCTG
GGCGGCCCCAGCGTGTTCCTGTTCCCCCCCAAGCCCAAGGACACCCTGATGATCAGCCGCACCCCCGAGGTG
ACCTGCGTGGTGGTGGACGTGAGCCACGAGGACCCCGAGGTGAAGTTCAACTGGTACGTGGACGGCGTGGAG
GTGCACAACGCCAAGACCAAGCCCCGCGAGGAGCAGTACAACAGCACCTACCGCGTGGTGAGCGTGCTGACC
GTGCTGCACCAGGACTGGCTGAACGGCAAGGAGTACAAGTGCAAGGTGAGCAACAAGGCCCTGCCCGCCCCC
ATCGAGAAGACCATCAGCAAGGCCAAGGGCCAGCCCCGCGAGCCCCAGGTGTACACCCTGCCCCCCAGCCGC
GACGAGCTGACCAAGAACCAGGTGAGCCTGACCTGCCTGGTGAAGGGCTTCTACCCCAGCGACATCGCCGTG
GAGTGGGAGAGCAACGGCCAGCCCGAGAACAACTACAAGACCACCCCCCCCGTGCTGGACAGCGACGGCAGC
TTCTTCCTGTACAGCAAGCTGACCGTGGACAAGAGCCGCTGGCAGCAGGGCAACGTGTTCAGCTGCAGCGTG
ATGCACGAGGCCCTGCACAACCACTACACCCAGAAGAGCCTGAGCCTGAGCCCCGGCTAACGAAGGAAACGA
GGAAGCGGAGAAGCCAGACACAAACAGAAAATTGTGGCACCGGTGAAACAGACTTTGAATTTTGACCTTCTC
AAGTTGGCGGGAGACGTCGAGTCCAACCCTGGGCCCATGAAACTGCTGCATGTCTTCCTCCTCTTCCTGTGC
TTCCACCTCCGTTTCTGTAAAGTCACCTACACTAGCCAGGAGGATCTGGTGGAGAAGAAATGCCTGGCCAAG
AAGTATACCCACCTGAGCTGCGACAAAGTGTTCTGCCAGCCCTGGCAACGCTGCATTGAAGGTACTTGTGTG
TGCAAGCTGCCCTACCAGTGCCCCAAGAACGGCACGGCCGTGTGTGCCACCAACAGGAGGAGCTTCCCCACC
TACTGCCAGCAGAAGAGCCTGGAATGCCTCCACCCTGGCACCAAGTTTCTGAACAACGGGACCTGCACAGCC
GAGGGGAAATTCAGCGTCTCCCTCAAGCACGGCAATACAGACTCCGAGGGCATTGTGGAAGTGAAGCTGGTG
GACCAGGACAAGACCATGTTCATCTGCAAAAGCAGCTGGTCCATGCGGGAGGCCAATGTCGCCTGCCTGGAC
CTGGGCTTCCAGCAGGGCGCTGATACACAGCGCCGCTTTAAACTCAGTGACCTCAGCATCAACAGCACTGAG
TGTCTGCACGTGCACTGCCGGGGCCTGGAGACCAGCCTGGCTGAGTGCACCTTCACCAAGCGCAGGACCATG
GGCTACCAGGATTTTGCAGATGTGGTCTGCTACACCCAGAAGGCAGACAGCCCCATGGATGACTTCTTCCAG
TGTGTCAATGGCAAGTACATTTCCCAGATGAAGGCTTGTGACGGGATCAATGATTGCGGGGATCAGAGCGAT
GAGCTCTGCTGCAAGGCCTGCCAAGGGAAGGGCTTTCACTGTAAGTCTGGGGTGTGCATCCCTTCTCAGTAT
CAGTGCAACGGAGAGGTGGACTGCATCACTGGGGAGGACGAGGTGGGCTGTGCTGGCTTCGCCTCTGTGGCC
CAGGAGGAGACAGAGATCCTCACAGCTGACATGGATGCAGAGCGGCGGCGCATCAAGAGTCTGCTCCCAAAG
CTCTCCTGCGGCGTTAAGAATCGCATGCACATCCGGAGGAAGCGGATCGTTGGAGGCAAACGGGCTCAGCTG
GGGGACTTGCCGTGGCAGGTGGCCATCAAAGATGCCTCCGGAATCACCTGTGGTGGCATCTACATCGGCGGC
TGCTGGATCCTGACCGCCGCCCACTGCCTTCGGGCCAGCAAGACTCACCGCTACCAGATCTGGACCACCGTG
GTGGATTGGATTCACCCCGACCTGAAGAGGATTGTCATTGAGTATGTCGACCGCATCATCTTCCATGAAAAC
TACAATGCCGGGACGTATCAGAACGACATCGCCCTCATCGAGATGAAGAAGGATGGGAACAAGAAGGACTGT
GAGCTGCCTCGCTCCATCCCCGCCTGTGTACCATGGTCTCCGTACCTGTTCCAGCCAAATGACACATGCATC
GTGAGCGGCTGGGGCCGCGAGAAAGACAACGAGAGGGTCTTCTCCCTGCAGTGGGGTGAAGTCAAGCTGATC
AGCAACTGCTCCAAGTTCTACGGCAACCGCTTCTATGAGAAGGAGATGGAGTGCGCCGGCACCTATGACGGC
AGCATTGACGCGTGCAAGGGAGACAGTGGGGGCCCCCTGGTCTGCATGGACGCCAACAATGTGACCTACGTG
TGGGGAGTTGTGTCCTGGGGCGAGAACTGTGGCAAGCCTGAGTTCCCGGGCGTGTACACAAAGGTGGCAAAC
TATTTTGACTGGATCTCCTATCACGTTGGCAGGCCCTTCATTTCACAGTACAACGTACTCGAGGTGCCTTCT
AGTTGCCAGCCATCTGTTGTTTGCCCCTCCCCCGTGCCTTCCTTGACCCTGGAAGGTGCCACTCCCACTGTC
CTTTCCTAATAAAATGAGGAAATTGCATCGCATTGTCTGAGTAGGTGTCATTCTATTCTGGGGGGTGGGGTG
GGGCAGGACAGCAAGGGGGAGGATTGGGAAGACAATAGCAGGCATGCTGGGGATGCGGTGGGCTCTATGGGC
GGCCGCAGGAACCCCTAGTGATGGAGTTGGCCACTCCCTCTCTGCGCGCTCGCTCGCTCACTGAGGCCGGGC
GACCAAAGGTCGCCCGACGCCCGGGCTTTGCCCGGGCGGCCTCAGTGAGCGAGCG

In some embodiments, the polynucleotide comprises or consists of the nucleotide sequence of SEQ ID NO: 49, or a nucleotide sequence that has at least 75%, 80%, 85%, 86%, 87%, 88%, 89%, 90%, 91%, 92%, 93%, 94%, 95%, 96%, 97%, 98% or 99% sequence identity thereto.

(SEQ ID NO: 49)
CGCTCGCTCACTGAGGCCGCCCGGGCAAAGCCCGGGCGTCGGGCGACCTTTGGTCGCCCGGCCTCAGTGAGCGAG
CGAGCGCGCAGAGAGGGAGTGGCCAACTCCATCACTAGGGGTTCCTGGCGCGCCGGAGTTCCGCGTTACATAACT
TACGGTAAATGGCCCGCCTGGCTGACCGCCCAACGACCCCCGCCCATTGACGTCAATAATGACGTATGTTCCCAT
AGTAACGCCAATAGGGACTTTCCATTGACGTCAATGGGTGGAGTATTTACGGTAAACTGCCCACTTGGCAGTACA
TCAAGTGTATCATATGCCAAGTACGCCCCCTATTGACGTCAATGACGGTAAATGGCCCGCCTGGCATTATGCCCA
GTACATGACCTTATGGGACTTTCCTACTTGGCAGTACATCTACGTATTAGTCATCGCTATTACCATGGTGATGCG
GTTTTGGCAGTACATCAATGGGCGTGGATAGCGGTTTGACTCACGGGGATTTCCAAGTCTCCACCCCATTGACGT
CAATGGGAGTTTGTTTTGGCACCAAAATCAACGGGACTTTCCAAAATGTCGTAACAACTCCGCCCCATTGACGCA
AATGGGCGGTAGGCGTGTACGGTGGGAGGTCTATATAAGCAGAGCTCGTTTAGTGAACCGTCAGATCGCCTGGAG
ACGCCATCCACGCTGTTTTGACCTCCATAGAAGACACCGACTAGTGCCACCATGCGCCTCCTGGCCAAGATCATC
TGCCTCATGCTGTGGGCCATCTGCGTGGCTGAGGACTGCAATGAGCTGCCGCCCAGGAGGAACACAGAGATCCTG
ACAGGGAGCTGGTCTGACCAGACCTACCCTGAGGGCACCCAGGCGATCTACAAGTGCCGGCCGGGCTACAGGAGC
CTGGGGAACATCATCATGGTGTGTAGAAAGGGCGAATGGGTGGCCCTCAACCCCCTGAGGAAGTGCCAGAAGCGG
CCCTGTGGCCACCCCGGGGACACACCCTTCGGGACCTTCACCCTGACCGGCGGCAATGTGTTTGAGTACGGCGTG
AAGGCTGTCTACACATGCAACGAGGGGTACCAGCTGCTGGGCGAGATTAACTACCGGGAGTGTGACACCGATGGG
TGGACCAACGACATTCCCATCTGTGAGGTGGTCAAGTGTCTCCCCGTGACAGCCCCAGAAAATGGCAAAATCGTG
AGCAGCGCCATGGAGCCTGACCGCGAATATCACTTTGGGCAGGCCGTGAGGTTTGTGTGCAACTCGGGCTACAAA
ATTGAAGGTGATGAGGAGATGCACTGCAGCGATGATGGCTTCTGGTCCAAGGAGAAGCCCAAATGTGTGGAGATC
TCCTGCAAGTCTCCCGACGTGATCAACGGCAGCCCAATCAGCCAGAAGATTATTTACAAAGAGAACGAGCGCTTC
CAGTACAAGTGTAACATGGGCTATGAGTATTCAGAGAGGGGAGATGCCGTCTGCACTGAGAGCGGCTGGAGACCA
CTGCCTAGCTGCGAGGAAAAGAGTTGTGACAACCCTTACATCCCAAATGGCGACTACTCCCCTCTGCGGATCAAA
CACCGGACCGGGGATGAAATCACCTATCAGTGCCGCAATGGATTCTACCCGGCCACCCGCGGCAACACCGCCAAA
TGCACCAGCACAGGCTGGATCCCCGCCCCCCGCTGTACGCTGAAGCCTTGCGACTATCCAGACATCAAGCACGGA
GGCCTGTACCACGAAAACATGCGGCGGCCTTATTTCCCTGTGGCAGTGGGGAAGTACTACAGCTACTACTGCGAC
GAGCACTTCGAGACCCCCTCTGGCTCCTACTGGGACCACATCCACTGCACACAGGACGGCTGGTCTCCAGCTGTG
CCCTGCCTGAGGAAATGCTACTTCCCCTACCTGGAGAACGGATACAACCAGAACTATGGCCGCAAGTTCGTGCAG
GGCAAGAGCATCGATGTGGCCTGCCACCCTGGCTACGCCCTGCCCAAGGCCCAGACAACTGTGACCTGCATGGAG
AATGGTTGGAGCCCCACCCCGCGCTGCATCCGGGTGTCCTTCACGCTCCGAAGGAAACGAGGAAGCGGAGAAGCC
AGACACAAACAGAAAATTGTGGCACCGGTGAAACAGACTTTGAATTTTGACCTTCTCAAGTTGGCGGGAGACGTC
GAGTCCAACCCTGGGCCCATGCGCCTGCTGGCCAAGATCATCTGCCTGATGCTGTGGGCCATCTGCGTGGCCAGC
GACACCGGCCGCCCCTTCGTGGAGATGTACAGCGAGATCCCCGAGATCATCCACATGACCGAGGGCCGCGAGCTG
GTGATCCCCTGCCGCGTGACCAGCCCCAACATCACCGTGACCCTGAAGAAGTTCCCCCTGGACACCCTGATCCCC
GACGGCAAGCGCATCATCTGGGACAGCCGCAAGGGCTTCATCATCAGCAACGCCACCTACAAGGAGATCGGCCTG
CTGACCTGCGAGGCCACCGTGAACGGCCACCTGTACAAGACCAACTACCTGACCCACCGCCAGACCAACACCATC
ATCGACGTGGTGCTGAGCCCCAGCCACGGCATCGAGCTGAGCGTGGGCGAGAAGCTGGTGCTGAACTGCACCGCC
CGCACCGAGCTGAACGTGGGCATCGACTTCAACTGGGAGTACCCCAGCAGCAAGCACCAGCACAAGAAGCTGGTG
AACCGCGACCTGAAGACCCAGAGCGGCAGCGAGATGAAGAAGTTCCTGAGCACCCTGACCATCGACGGCGTGACC
CGCAGCGACCAGGGCCTGTACACCTGCGCCGCCAGCAGCGGCCTGATGACCAAGAAGAACAGCACCTTCGTGCGC
GTGCACGAGAAGGACAAGACCCACACCTGCCCCCCCTGCCCCGCCCCCGAGCTGCTGGGCGGCCCCAGCGTGTTC
CTGTTCCCCCCCAAGCCCAAGGACACCCTGATGATCAGCCGCACCCCCGAGGTGACCTGCGTGGTGGTGGACGTG
AGCCACGAGGACCCCGAGGTGAAGTTCAACTGGTACGTGGACGGCGTGGAGGTGCACAACGCCAAGACCAAGCCC
CGCGAGGAGCAGTACAACAGCACCTACCGCGTGGTGAGCGTGCTGACCGTGCTGCACCAGGACTGGCTGAACGGC
AAGGAGTACAAGTGCAAGGTGAGCAACAAGGCCCTGCCCGCCCCCATCGAGAAGACCATCAGCAAGGCCAAGGGC
CAGCCCCGCGAGCCCCAGGTGTACACCCTGCCCCCCAGCCGCGACGAGCTGACCAAGAACCAGGTGAGCCTGACC
TGCCTGGTGAAGGGCTTCTACCCCAGCGACATCGCCGTGGAGTGGGAGAGCAACGGCCAGCCCGAGAACAACTAC
AAGACCACCCCCCCCGTGCTGGACAGCGACGGCAGCTTCTTCCTGTACAGCAAGCTGACCGTGGACAAGAGCCGC
TGGCAGCAGGGCAACGTGTTCAGCTGCAGCGTGATGCACGAGGCCCTGCACAACCACTACACCCAGAAGAGCCTG
AGCCTGAGCCCCGGCTAACTCGAGAATCAACCTCTGGATTACAAAATTTGTGAAAGATTGACTGGTATTCTTAAC
TATGTTGCTCCTTTTACGCTATGTGGATACGCTGCTTTAATGCCTTTGTATCATGCTATTGCTTCCCGTATGGCT
TTCATTTTCTCCTCCTTGTATAAATCCTGGTTGCTGTCTCTTTATGAGGAGTTGTGGCCCGTTGTCAGGCAACGT
GGCGTGGTGTGCACTGTGTTTGCTGACGCAACCCCCACTGGTTGGGGCATTGCCACCACCTGTCAGCTCCTTTCC
GGGACTTTCGCTTTCCCCCTCCCTATTGCCACGGCGGAACTCATCGCCGCCTGCCTTGCCCGCTGCTGGACAGGG
GCTCGGCTGTTGGGCACTGACAATTCCGTGGTGTTGTCGGGGAAATCATCGTCCTTTCCTTGGCTGCTCGCCTGT
GTTGCCACCTGGATTCTGCGCGGGACGTCCTTCTGCTACGTCCCTTCGGCCCTCAATCCAGCGGACCTTCCTTCC
CGCGGCCTGCTGCCGGCTCTGCGGCCTCTTCCGCGTCTTCGCCTTCGCCCTCAGACGAGTCGGATCTCCCTTTGG
GCCGCCTCCCCGCATCGATACCGTCGACTCGCTGATCAGCCTGTGCCTTCTAGTTGCCAGCCATCTGTTGTTTGC
CCCTCCCCCGTGCCTTCCTTGACCCTGGAAGGTGCCACTCCCACTGTCCTTTCCTAATAAAATGAGGAAATTGCA
TCGCATTGTCTGAGTAGGTGTCATTCTATTCTGGGGGGTGGGGTGGGGCAGGACAGCAAGGGGGAGGATTGGGAA
GACAATAGCAGGCATGCTGGGGATGCGGTGGGCTCTATGGGCGGCCGCAGGAACCCCTAGTGATGGAGTTGGCCA
CTCCCTCTCTGCGCGCTCGCTCGCTCACTGAGGCCGGGCGACCAAAGGTCGCCCGACGCCCGGGCTTTGCCCGGG
CGGCCTCAGTGAGCGAGCG

In some embodiments, the polynucleotide comprises or consists of the nucleotide sequence of SEQ ID NO: 50, or a nucleotide sequence that has at least 75%, 80%, 85%, 86%, 87%, 88%, 89%, 90%, 91%, 92%, 93%, 94%, 95%, 96%, 97%, 98% or 99% sequence identity thereto.

(SEQ ID NO: 50)
CGCTCGCTCACTGAGGCCGCCCGGGCAAAGCCCGGGCGTCGGGCGACCTTTGGTCGCCCGGCCTCAGTGAGC
GAGCGAGCGCGCAGAGAGGGAGTGGCCAACTCCATCACTAGGGGTTCCTGGCGCGCCGGAGTTCCGCGTTAC
ATAACTTACGGTAAATGGCCCGCCTGGCTGACCGCCCAACGACCCCCGCCCATTGACGTCAATAATGACGTA
TGTTCCCATAGTAACGCCAATAGGGACTTTCCATTGACGTCAATGGGTGGAGTATTTACGGTAAACTGCCCA
CTTGGCAGTACATCAAGTGTATCATATGCCAAGTACGCCCCCTATTGACGTCAATGACGGTAAATGGCCCGC
CTGGCATTATGCCCAGTACATGACCTTATGGGACTTTCCTACTTGGCAGTACATCTACGTATTAGTCATCGC
TATTACCATGGTGATGCGGTTTTGGCAGTACATCAATGGGCGTGGATAGCGGTTTGACTCACGGGGATTTCC
AAGTCTCCACCCCATTGACGTCAATGGGAGTTTGTTTTGGCACCAAAATCAACGGGACTTTCCAAAATGTCG
TAACAACTCCGCCCCATTGACGCAAATGGGCGGTAGGCGTGTACGGTGGGAGGTCTATATAAGCAGAGCTCG
TTTAGTGAACCGTCAGATCGCCTGGAGACGCCATCCACGCTGTTTTGACCTCCATAGAAGACACCGACTAGT
GCCACCATGCGCCTGCTGGCCAAGATCATCTGCCTGATGCTGTGGGCCATCTGCGTGGCCAGCGACACCGGC
CGCCCCTTCGTGGAGATGTACAGCGAGATCCCCGAGATCATCCACATGACCGAGGGCCGCGAGCTGGTGATC
CCCTGCCGCGTGACCAGCCCCAACATCACCGTGACCCTGAAGAAGTTCCCCCTGGACACCCTGATCCCCGAC
GGCAAGCGCATCATCTGGGACAGCCGCAAGGGCTTCATCATCAGCAACGCCACCTACAAGGAGATCGGCCTG
CTGACCTGCGAGGCCACCGTGAACGGCCACCTGTACAAGACCAACTACCTGACCCACCGCCAGACCAACACC
ATCATCGACGTGGTGCTGAGCCCCAGCCACGGCATCGAGCTGAGCGTGGGCGAGAAGCTGGTGCTGAACTGC
ACCGCCCGCACCGAGCTGAACGTGGGCATCGACTTCAACTGGGAGTACCCCAGCAGCAAGCACCAGCACAAG
AAGCTGGTGAACCGCGACCTGAAGACCCAGAGCGGCAGCGAGATGAAGAAGTTCCTGAGCACCCTGACCATC
GACGGCGTGACCCGCAGCGACCAGGGCCTGTACACCTGCGCCGCCAGCAGCGGCCTGATGACCAAGAAGAAC
AGCACCTTCGTGCGCGTGCACGAGAAGGACAAGACCCACACCTGCCCCCCCTGCCCCGCCCCCGAGCTGCTG
GGCGGCCCCAGCGTGTTCCTGTTCCCCCCCAAGCCCAAGGACACCCTGATGATCAGCCGCACCCCCGAGGTG
ACCTGCGTGGTGGTGGACGTGAGCCACGAGGACCCCGAGGTGAAGTTCAACTGGTACGTGGACGGCGTGGAG
GTGCACAACGCCAAGACCAAGCCCCGCGAGGAGCAGTACAACAGCACCTACCGCGTGGTGAGCGTGCTGACC
GTGCTGCACCAGGACTGGCTGAACGGCAAGGAGTACAAGTGCAAGGTGAGCAACAAGGCCCTGCCCGCCCCC
ATCGAGAAGACCATCAGCAAGGCCAAGGGCCAGCCCCGCGAGCCCCAGGTGTACACCCTGCCCCCCAGCCGC
GACGAGCTGACCAAGAACCAGGTGAGCCTGACCTGCCTGGTGAAGGGCTTCTACCCCAGCGACATCGCCGTG
GAGTGGGAGAGCAACGGCCAGCCCGAGAACAACTACAAGACCACCCCCCCCGTGCTGGACAGCGACGGCAGC
TTCTTCCTGTACAGCAAGCTGACCGTGGACAAGAGCCGCTGGCAGCAGGGCAACGTGTTCAGCTGCAGCGTG
ATGCACGAGGCCCTGCACAACCACTACACCCAGAAGAGCCTGAGCCTGAGCCCCGGCTAACGAAGGAAACGA
GGAAGCGGAGAAGCCAGACACAAACAGAAAATTGTGGCACCGGTGAAACAGACTTTGAATTTTGACCTTCTC
AAGTTGGCGGGAGACGTCGAGTCCAACCCTGGGCCCATGCGCCTCCTGGCCAAGATCATCTGCCTCATGCTG
TGGGCCATCTGCGTGGCTGAGGACTGCAATGAGCTGCCGCCCAGGAGGAACACAGAGATCCTGACAGGGAGC
TGGTCTGACCAGACCTACCCTGAGGGCACCCAGGCGATCTACAAGTGCCGGCCGGGCTACAGGAGCCTGGGG
AACATCATCATGGTGTGTAGAAAGGGCGAATGGGTGGCCCTCAACCCCCTGAGGAAGTGCCAGAAGCGGCCC
TGTGGCCACCCCGGGGACACACCCTTCGGGACCTTCACCCTGACCGGCGGCAATGTGTTTGAGTACGGCGTG
AAGGCTGTCTACACATGCAACGAGGGGTACCAGCTGCTGGGCGAGATTAACTACCGGGAGTGTGACACCGAT
GGGTGGACCAACGACATTCCCATCTGTGAGGTGGTCAAGTGTCTCCCCGTGACAGCCCCAGAAAATGGCAAA
ATCGTGAGCAGCGCCATGGAGCCTGACCGCGAATATCACTTTGGGCAGGCCGTGAGGTTTGTGTGCAACTCG
GGCTACAAAATTGAAGGTGATGAGGAGATGCACTGCAGCGATGATGGCTTCTGGTCCAAGGAGAAGCCCAAA
TGTGTGGAGATCTCCTGCAAGTCTCCCGACGTGATCAACGGCAGCCCAATCAGCCAGAAGATTATTTACAAA
GAGAACGAGCGCTTCCAGTACAAGTGTAACATGGGCTATGAGTATTCAGAGAGGGGAGATGCCGTCTGCACT
GAGAGCGGCTGGAGACCACTGCCTAGCTGCGAGGAAAAGAGTTGTGACAACCCTTACATCCCAAATGGCGAC
TACTCCCCTCTGCGGATCAAACACCGGACCGGGGATGAAATCACCTATCAGTGCCGCAATGGATTCTACCCG
GCCACCCGCGGCAACACCGCCAAATGCACCAGCACAGGCTGGATCCCCGCCCCCCGCTGTACGCTGAAGCCT
TGCGACTATCCAGACATCAAGCACGGAGGCCTGTACCACGAAAACATGCGGCGGCCTTATTTCCCTGTGGCA
GTGGGGAAGTACTACAGCTACTACTGCGACGAGCACTTCGAGACCCCCTCTGGCTCCTACTGGGACCACATC
CACTGCACACAGGACGGCTGGTCTCCAGCTGTGCCCTGCCTGAGGAAATGCTACTTCCCCTACCTGGAGAAC
GGATACAACCAGAACTATGGCCGCAAGTTCGTGCAGGGCAAGAGCATCGATGTGGCCTGCCACCCTGGCTAC
GCCCTGCCCAAGGCCCAGACAACTGTGACCTGCATGGAGAATGGTTGGAGCCCCACCCCGCGCTGCATCCGG
GTGTCCTTCACGCTCCTCGAGAATCAACCTCTGGATTACAAAATTTGTGAAAGATTGACTGGTATTCTTAAC
TATGTTGCTCCTTTTACGCTATGTGGATACGCTGCTTTAATGCCTTTGTATCATGCTATTGCTTCCCGTATG
GCTTTCATTTTCTCCTCCTTGTATAAATCCTGGTTGCTGTCTCTTTATGAGGAGTTGTGGCCCGTTGTCAGG
CAACGTGGCGTGGTGTGCACTGTGTTTGCTGACGCAACCCCCACTGGTTGGGGCATTGCCACCACCTGTCAG
CTCCTTTCCGGGACTTTCGCTTTCCCCCTCCCTATTGCCACGGCGGAACTCATCGCCGCCTGCCTTGCCCGC
TGCTGGACAGGGGCTCGGCTGTTGGGCACTGACAATTCCGTGGTGTTGTCGGGGAAATCATCGTCCTTTCCT
TGGCTGCTCGCCTGTGTTGCCACCTGGATTCTGCGCGGGACGTCCTTCTGCTACGTCCCTTCGGCCCTCAAT
CCAGCGGACCTTCCTTCCCGCGGCCTGCTGCCGGCTCTGCGGCCTCTTCCGCGTCTTCGCCTTCGCCCTCAG
ACGAGTCGGATCTCCCTTTGGGCCGCCTCCCCGCATCGATACCGTCGACTCGCTGATCAGCCTGTGCCTTCT
AGTTGCCAGCCATCTGTTGTTTGCCCCTCCCCCGTGCCTTCCTTGACCCTGGAAGGTGCCACTCCCACTGTC
CTTTCCTAATAAAATGAGGAAATTGCATCGCATTGTCTGAGTAGGTGTCATTCTATTCTGGGGGGTGGGGTG
GGGCAGGACAGCAAGGGGGAGGATTGGGAAGACAATAGCAGGCATGCTGGGGATGCGGTGGGCTCTATGGGC
GGCCGCAGGAACCCCTAGTGATGGAGTTGGCCACTCCCTCTCTGCGCGCTCGCTCGCTCACTGAGGCCGGGC
GACCAAAGGTCGCCCGACGCCCGGGCTTTGCCCGGGCGGCCTCAGTGAGCGAGCG

In some embodiments, the polynucleotide comprises or consists of the nucleotide sequence of SEQ ID NO: 51, or a nucleotide sequence that has at least 75%, 80%, 85%, 86%, 87%, 88%, 89%, 90%, 91%, 92%, 93%, 94%, 95%, 96%, 97%, 98% or 99% sequence identity thereto.

(SEQ ID NO: 51)
CGCTCGCTCACTGAGGCCGCCCGGGCAAAGCCCGGGCGTCGGGCGACCTTTGGTCGCCCGGCCTCAGTGAGC
GAGCGAGCGCGCAGAGAGGGAGTGGCCAACTCCATCACTAGGGGTTCCTGGCGCGCCCCATTGACGTCAATA
ATGACGTATGTTCCCATAGTAACGCCAATAGGGACTTTCCATTGACGTCAATGGGTGGAGTATTTACGGTAA
ACTGCCCACTTGGCAGTACATCAAGTGTATCATATGCCAAGTACGCCCCCTATTGACGTCAATGACGGTAAA
TGGCCCGCCTGGCATTATGCCCAGTACATGACCTTATGGGACTTTCCTACTTGGCAGTACATCTACGTATTA
GTCATCGCTATTACCATGGTCGAGGTGAGCCCCACGTTCTGCTTCACTCTCCCCATCTCCCCCCCCTCCCCA
CCCCCAATTTTGTATTTATTTATTTTTTAATTATTTTGTGCAGCGATGGGGGCGGGGGGGGGGGGGGGGCGC
GCGCCAGGCGGGGCGGGGCGGGGCGAGGGGCGGGGCGGGGCGAGGCGGAGAGGTGCGGCGGCAGCCAATCAG
AGCGGCGCGCTCCGAAAGTTTCCTTTTATGGCGAGGCGGCGGCGGCGGCGGCCCTATAAAAAGCGAAGCGCG
CGGCGGGCGGGAGTCGCTGCGCGCTGCCTTCGCCCCGTGCCCCGCTCCGCCGCCGCCTCGCGCCGCCCGCCC
CGGCTCTGACTGACCGCGTTACTCCCACAGGTGAGCGGGCGGGACGGCCCTTCTCCTCCGGGCTGTAATTAG
CGCTTGGTTTAATGACGGCTTGTTTCTTTTCTGTGGCTGCGTGAAAGCCTTGAGGGGCTCCGGGAGGGCCCT
TTGTGCGGGGGGAGCGGCTCGGGGCTGTCCGCGGGGGGACGGCTGCCTTCGGGGGGGACGGGGCAGGGCGGG
GTTCGGCTTCTGGCGTGTGACCGGCGGCTCTAGAGCCTCTGCTAACCATGTTCATGCCTTCTTCTTTTTCCT
ACAGCTCCTGGGCAACGTGCTGGTTATTGTGCTGTCTCATCATTTTGGCAAAGAATTGGATCCACTAGTGCC
ACCATGCGCCTCCTGGCCAAGATCATCTGCCTCATGCTGTGGGCCATCTGCGTGGCTGAGGACTGCAATGAG
CTGCCGCCCAGGAGGAACACAGAGATCCTGACAGGGAGCTGGTCTGACCAGACCTACCCTGAGGGCACCCAG
GCGATCTACAAGTGCCGGCCGGGCTACAGGAGCCTGGGGAACATCATCATGGTGTGTAGAAAGGGCGAATGG
GTGGCCCTCAACCCCCTGAGGAAGTGCCAGAAGCGGCCCTGTGGCCACCCCGGGGACACACCCTTCGGGACC
TTCACCCTGACCGGCGGCAATGTGTTTGAGTACGGCGTGAAGGCTGTCTACACATGCAACGAGGGGTACCAG
CTGCTGGGCGAGATTAACTACCGGGAGTGTGACACCGATGGGTGGACCAACGACATTCCCATCTGTGAGGTG
GTCAAGTGTCTCCCCGTGACAGCCCCAGAAAATGGCAAAATCGTGAGCAGCGCCATGGAGCCTGACCGCGAA
TATCACTTTGGGCAGGCCGTGAGGTTTGTGTGCAACTCGGGCTACAAAATTGAAGGTGATGAGGAGATGCAC
TGCAGCGATGATGGCTTCTGGTCCAAGGAGAAGCCCAAATGTGTGGAGATCTCCTGCAAGTCTCCCGACGTG
ATCAACGGCAGCCCAATCAGCCAGAAGATTATTTACAAAGAGAACGAGCGCTTCCAGTACAAGTGTAACATG
GGCTATGAGTATTCAGAGAGGGGAGATGCCGTCTGCACTGAGAGCGGCTGGAGACCACTGCCTAGCTGCGAG
GAAAAGAGTTGTGACAACCCTTACATCCCAAATGGCGACTACTCCCCTCTGCGGATCAAACACCGGACCGGG
GATGAAATCACCTATCAGTGCCGCAATGGATTCTACCCGGCCACCCGCGGCAACACCGCCAAATGCACCAGC
ACAGGCTGGATCCCCGCCCCCCGCTGTACGCTGAAGCCTTGCGACTATCCAGACATCAAGCACGGAGGCCTG
TACCACGAAAACATGCGGCGGCCTTATTTCCCTGTGGCAGTGGGGAAGTACTACAGCTACTACTGCGACGAG
CACTTCGAGACCCCCTCTGGCTCCTACTGGGACCACATCCACTGCACACAGGACGGCTGGTCTCCAGCTGTG
CCCTGCCTGAGGAAATGCTACTTCCCCTACCTGGAGAACGGATACAACCAGAACTATGGCCGCAAGTTCGTG
CAGGGCAAGAGCATCGATGTGGCCTGCCACCCTGGCTACGCCCTGCCCAAGGCCCAGACAACTGTGACCTGC
ATGGAGAATGGTTGGAGCCCCACCCCGCGCTGCATCCGGGTGTCCTTCACGCTCCGAAGGAAACGAGGAAGC
GGAGAAGCCAGACACAAACAGAAAATTGTGGCACCGGTGAAACAGACTTTGAATTTTGACCTTCTCAAGTTG
GCGGGAGACGTCGAGTCCAACCCTGGGCCCATGCGCCTGCTGGCCAAGATCATCTGCCTGATGCTGTGGGCC
ATCTGCGTGGCCAGCGACACCGGCCGCCCCTTCGTGGAGATGTACAGCGAGATCCCCGAGATCATCCACATG
ACCGAGGGCCGCGAGCTGGTGATCCCCTGCCGCGTGACCAGCCCCAACATCACCGTGACCCTGAAGAAGTTC
CCCCTGGACACCCTGATCCCCGACGGCAAGCGCATCATCTGGGACAGCCGCAAGGGCTTCATCATCAGCAAC
GCCACCTACAAGGAGATCGGCCTGCTGACCTGCGAGGCCACCGTGAACGGCCACCTGTACAAGACCAACTAC
CTGACCCACCGCCAGACCAACACCATCATCGACGTGGTGCTGAGCCCCAGCCACGGCATCGAGCTGAGCGTG
GGCGAGAAGCTGGTGCTGAACTGCACCGCCCGCACCGAGCTGAACGTGGGCATCGACTTCAACTGGGAGTAC
CCCAGCAGCAAGCACCAGCACAAGAAGCTGGTGAACCGCGACCTGAAGACCCAGAGCGGCAGCGAGATGAAG
AAGTTCCTGAGCACCCTGACCATCGACGGCGTGACCCGCAGCGACCAGGGCCTGTACACCTGCGCCGCCAGC
AGCGGCCTGATGACCAAGAAGAACAGCACCTTCGTGCGCGTGCACGAGAAGGACAAGACCCACACCTGCCCC
CCCTGCCCCGCCCCCGAGCTGCTGGGCGGCCCCAGCGTGTTCCTGTTCCCCCCCAAGCCCAAGGACACCCTG
ATGATCAGCCGCACCCCCGAGGTGACCTGCGTGGTGGTGGACGTGAGCCACGAGGACCCCGAGGTGAAGTTC
AACTGGTACGTGGACGGCGTGGAGGTGCACAACGCCAAGACCAAGCCCCGCGAGGAGCAGTACAACAGCACC
TACCGCGTGGTGAGCGTGCTGACCGTGCTGCACCAGGACTGGCTGAACGGCAAGGAGTACAAGTGCAAGGTG
AGCAACAAGGCCCTGCCCGCCCCCATCGAGAAGACCATCAGCAAGGCCAAGGGCCAGCCCCGCGAGCCCCAG
GTGTACACCCTGCCCCCCAGCCGCGACGAGCTGACCAAGAACCAGGTGAGCCTGACCTGCCTGGTGAAGGGC
TTCTACCCCAGCGACATCGCCGTGGAGTGGGAGAGCAACGGCCAGCCCGAGAACAACTACAAGACCACCCCC
CCCGTGCTGGACAGCGACGGCAGCTTCTTCCTGTACAGCAAGCTGACCGTGGACAAGAGCCGCTGGCAGCAG
GGCAACGTGTTCAGCTGCAGCGTGATGCACGAGGCCCTGCACAACCACTACACCCAGAAGAGCCTGAGCCTG
AGCCCCGGCTAACTCGAGAATCAACCTCTGGATTACAAAATTTGTGAAAGATTGACTGGTATTCTTAACTAT
GTTGCTCCTTTTACGCTATGTGGATACGCTGCTTTAATGCCTTTGTATCATGCTATTGCTTCCCGTATGGCT
TTCATTTTCTCCTCCTTGTATAAATCCTGGTTAGTTCTTGCCACGGCGGAACTCATCGCCGCCTGCCTTGCC
CGCTGCTGGACAGGGGCTCGGCTGTTGGGCACTGACAATTCCGTGGTGTGCCTTCTAGTTGCCAGCCATCTG
TTGTTTGCCCCTCCCCCGTGCCTTCCTTGACCCTGGAAGGTGCCACTCCCACTGTCCTTTCCTAATAAAATG
AGGAAATTGCATCGCATTGTCTGAGTAGGTGTCATTCTATTCTGGGGGGTGGGGTGGGGCAGGACAGCAAGG
GGGAGGATTGGGAAGACAATAGCAGGCATGCTGGGGATGCGGTGGGCTCTATGGGCGGCCGCAGGAACCCCT
AGTGATGGAGTTGGCCACTCCCTCTCTGCGCGCTCGCTCGCTCACTGAGGCCGGGCGACCAAAGGTCGCCCG
ACGCCCGGGCTTTGCCCGGGCGGCCTCAGTGAGCGAGCG

In some embodiments, the polynucleotide comprises or consists of the nucleotide sequence of SEQ ID NO: 52, or a nucleotide sequence that has at least 75%, 80%, 85%, 86%, 87%, 88%, 89%, 90%, 91%, 92%, 93%, 94%, 95%, 96%, 97%, 98% or 99% sequence identity thereto.

(SEQ ID NO: 52)
CGCTCGCTCACTGAGGCCGCCCGGGCAAAGCCCGGGCGTCGGGCGACCTTTGGTCGCCCGGCCTCAGTGAGCGAG
CGAGCGCGCAGAGAGGGAGTGGCCAACTCCATCACTAGGGGTTCCTGGCGCGCCCCATTGACGTCAATAATGACG
TATGTTCCCATAGTAACGCCAATAGGGACTTTCCATTGACGTCAATGGGTGGAGTATTTACGGTAAACTGCCCAC
TTGGCAGTACATCAAGTGTATCATATGCCAAGTACGCCCCCTATTGACGTCAATGACGGTAAATGGCCCGCCTGG
CATTATGCCCAGTACATGACCTTATGGGACTTTCCTACTTGGCAGTACATCTACGTATTAGTCATCGCTATTACC
ATGGTCGAGGTGAGCCCCACGTTCTGCTTCACTCTCCCCATCTCCCCCCCCTCCCCACCCCCAATTTTGTATTTA
TTTATTTTTTAATTATTTTGTGCAGCGATGGGGGCGGGGGGGGGGGGGGGGCGCGCGCCAGGCGGGGCGGGGCGG
GGCGAGGGGCGGGGCGGGGCGAGGCGGAGAGGTGCGGCGGCAGCCAATCAGAGCGGCGCGCTCCGAAAGTTTCCT
TTTATGGCGAGGCGGCGGCGGCGGCGGCCCTATAAAAAGCGAAGCGCGCGGCGGGCGGGAGTCGCTGCGCGCTGC
CTTCGCCCCGTGCCCCGCTCCGCCGCCGCCTCGCGCCGCCCGCCCCGGCTCTGACTGACCGCGTTACTCCCACAG
GTGAGCGGGCGGGACGGCCCTTCTCCTCCGGGCTGTAATTAGCGCTTGGTTTAATGACGGCTTGTTTCTTTTCTG
TGGCTGCGTGAAAGCCTTGAGGGGCTCCGGGAGGGCCCTTTGTGCGGGGGGAGCGGCTCGGGGCTGTCCGCGGGG
GGACGGCTGCCTTCGGGGGGGACGGGGCAGGGCGGGGTTCGGCTTCTGGCGTGTGACCGGCGGCTCTAGAGCCTC
TGCTAACCATGTTCATGCCTTCTTCTTTTTCCTACAGCTCCTGGGCAACGTGCTGGTTATTGTGCTGTCTCATCA
TTTTGGCAAAGAATTGGATCCACTAGTGCCACCATGCGCCTGCTGGCCAAGATCATCTGCCTGATGCTGTGGGCC
ATCTGCGTGGCCAGCGACACCGGCCGCCCCTTCGTGGAGATGTACAGCGAGATCCCCGAGATCATCCACATGACC
GAGGGCCGCGAGCTGGTGATCCCCTGCCGCGTGACCAGCCCCAACATCACCGTGACCCTGAAGAAGTTCCCCCTG
GACACCCTGATCCCCGACGGCAAGCGCATCATCTGGGACAGCCGCAAGGGCTTCATCATCAGCAACGCCACCTAC
AAGGAGATCGGCCTGCTGACCTGCGAGGCCACCGTGAACGGCCACCTGTACAAGACCAACTACCTGACCCACCGC
CAGACCAACACCATCATCGACGTGGTGCTGAGCCCCAGCCACGGCATCGAGCTGAGCGTGGGCGAGAAGCTGGTG
CTGAACTGCACCGCCCGCACCGAGCTGAACGTGGGCATCGACTTCAACTGGGAGTACCCCAGCAGCAAGCACCAG
CACAAGAAGCTGGTGAACCGCGACCTGAAGACCCAGAGCGGCAGCGAGATGAAGAAGTTCCTGAGCACCCTGACC
ATCGACGGCGTGACCCGCAGCGACCAGGGCCTGTACACCTGCGCCGCCAGCAGCGGCCTGATGACCAAGAAGAAC
AGCACCTTCGTGCGCGTGCACGAGAAGGACAAGACCCACACCTGCCCCCCCTGCCCCGCCCCCGAGCTGCTGGGC
GGCCCCAGCGTGTTCCTGTTCCCCCCCAAGCCCAAGGACACCCTGATGATCAGCCGCACCCCCGAGGTGACCTGC
GTGGTGGTGGACGTGAGCCACGAGGACCCCGAGGTGAAGTTCAACTGGTACGTGGACGGCGTGGAGGTGCACAAC
GCCAAGACCAAGCCCCGCGAGGAGCAGTACAACAGCACCTACCGCGTGGTGAGCGTGCTGACCGTGCTGCACCAG
GACTGGCTGAACGGCAAGGAGTACAAGTGCAAGGTGAGCAACAAGGCCCTGCCCGCCCCCATCGAGAAGACCATC
AGCAAGGCCAAGGGCCAGCCCCGCGAGCCCCAGGTGTACACCCTGCCCCCCAGCCGCGACGAGCTGACCAAGAAC
CAGGTGAGCCTGACCTGCCTGGTGAAGGGCTTCTACCCCAGCGACATCGCCGTGGAGTGGGAGAGCAACGGCCAG
CCCGAGAACAACTACAAGACCACCCCCCCCGTGCTGGACAGCGACGGCAGCTTCTTCCTGTACAGCAAGCTGACC
GTGGACAAGAGCCGCTGGCAGCAGGGCAACGTGTTCAGCTGCAGCGTGATGCACGAGGCCCTGCACAACCACTAC
ACCCAGAAGAGCCTGAGCCTGAGCCCCGGCTAACGAAGGAAACGAGGAAGCGGAGAAGCCAGACACAAACAGAAA
ATTGTGGCACCGGTGAAACAGACTTTGAATTTTGACCTTCTCAAGTTGGCGGGAGACGTCGAGTCCAACCCTGGG
CCCATGCGCCTCCTGGCCAAGATCATCTGCCTCATGCTGTGGGCCATCTGCGTGGCTGAGGACTGCAATGAGCTG
CCGCCCAGGAGGAACACAGAGATCCTGACAGGGAGCTGGTCTGACCAGACCTACCCTGAGGGCACCCAGGCGATC
TACAAGTGCCGGCCGGGCTACAGGAGCCTGGGGAACATCATCATGGTGTGTAGAAAGGGCGAATGGGTGGCCCTC
AACCCCCTGAGGAAGTGCCAGAAGCGGCCCTGTGGCCACCCCGGGGACACACCCTTCGGGACCTTCACCCTGACC
GGCGGCAATGTGTTTGAGTACGGCGTGAAGGCTGTCTACACATGCAACGAGGGGTACCAGCTGCTGGGCGAGATT
AACTACCGGGAGTGTGACACCGATGGGTGGACCAACGACATTCCCATCTGTGAGGTGGTCAAGTGTCTCCCCGTG
ACAGCCCCAGAAAATGGCAAAATCGTGAGCAGCGCCATGGAGCCTGACCGCGAATATCACTTTGGGCAGGCCGTG
AGGTTTGTGTGCAACTCGGGCTACAAAATTGAAGGTGATGAGGAGATGCACTGCAGCGATGATGGCTTCTGGTCC
AAGGAGAAGCCCAAATGTGTGGAGATCTCCTGCAAGTCTCCCGACGTGATCAACGGCAGCCCAATCAGCCAGAAG
ATTATTTACAAAGAGAACGAGCGCTTCCAGTACAAGTGTAACATGGGCTATGAGTATTCAGAGAGGGGAGATGCC
GTCTGCACTGAGAGCGGCTGGAGACCACTGCCTAGCTGCGAGGAAAAGAGTTGTGACAACCCTTACATCCCAAAT
GGCGACTACTCCCCTCTGCGGATCAAACACCGGACCGGGGATGAAATCACCTATCAGTGCCGCAATGGATTCTAC
CCGGCCACCCGCGGCAACACCGCCAAATGCACCAGCACAGGCTGGATCCCCGCCCCCCGCTGTACGCTGAAGCCT
TGCGACTATCCAGACATCAAGCACGGAGGCCTGTACCACGAAAACATGCGGCGGCCTTATTTCCCTGTGGCAGTG
GGGAAGTACTACAGCTACTACTGCGACGAGCACTTCGAGACCCCCTCTGGCTCCTACTGGGACCACATCCACTGC
ACACAGGACGGCTGGTCTCCAGCTGTGCCCTGCCTGAGGAAATGCTACTTCCCCTACCTGGAGAACGGATACAAC
CAGAACTATGGCCGCAAGTTCGTGCAGGGCAAGAGCATCGATGTGGCCTGCCACCCTGGCTACGCCCTGCCCAAG
GCCCAGACAACTGTGACCTGCATGGAGAATGGTTGGAGCCCCACCCCGCGCTGCATCCGGGTGTCCTTCACGCTC
CTCGAGAATCAACCTCTGGATTACAAAATTTGTGAAAGATTGACTGGTATTCTTAACTATGTTGCTCCTTTTACG
CTATGTGGATACGCTGCTTTAATGCCTTTGTATCATGCTATTGCTTCCCGTATGGCTTTCATTTTCTCCTCCTTG
TATAAATCCTGGTTAGTTCTTGCCACGGCGGAACTCATCGCCGCCTGCCTTGCCCGCTGCTGGACAGGGGCTCGG
CTGTTGGGCACTGACAATTCCGTGGTGTGCCTTCTAGTTGCCAGCCATCTGTTGTTTGCCCCTCCCCCGTGCCTT
CCTTGACCCTGGAAGGTGCCACTCCCACTGTCCTTTCCTAATAAAATGAGGAAATTGCATCGCATTGTCTGAGTA
GGTGTCATTCTATTCTGGGGGGTGGGGTGGGGCAGGACAGCAAGGGGGAGGATTGGGAAGACAATAGCAGGCATG
CTGGGGATGCGGTGGGCTCTATGGGCGGCCGCAGGAACCCCTAGTGATGGAGTTGGCCACTCCCTCTCTGCGCGC
TCGCTCGCTCACTGAGGCCGGGCGACCAAAGGTCGCCCGACGCCCGGGCTTTGCCCGGGCGGCCTCAGTGAGCGA
GCG

In some embodiments, the polynucleotide comprises or consists of the nucleotide sequence of SEQ ID NO: 53, or a nucleotide sequence that has at least 75%, 80%, 85%, 86%, 87%, 88%, 89%, 90%, 91%, 92%, 93%, 94%, 95%, 96%, 97%, 98% or 99% sequence identity thereto.

(SEQ ID NO: 53)
CGCTCGCTCACTGAGGCCGCCCGGGCAAAGCCCGGGCGTCGGGCGACCTTTGGTCGCCCGGCCTCAGTGAGCGAG
CGAGCGCGCAGAGAGGGAGTGGCCAACTCCATCACTAGGGGTTCCTGGCGCGCCCCATTGACGTCAATAATGACG
TATGTTCCCATAGTAACGCCAATAGGGACTTTCCATTGACGTCAATGGGTGGAGTATTTACGGTAAACTGCCCAC
TTGGCAGTACATCAAGTGTATCATATGCCAAGTACGCCCCCTATTGACGTCAATGACGGTAAATGGCCCGCCTGG
CATTATGCCCAGTACATGACCTTATGGGACTTTCCTACTTGGCAGTACATCTACGTATTAGTCATCGCTATTACC
ATGGTCGAGGTGAGCCCCACGTTCTGCTTCACTCTCCCCATCTCCCCCCCCTCCCCACCCCCAATTTTGTATTTA
TTTATTTTTTAATTATTTTGTGCAGCGATGGGGGCGGGGGGGGGGGGGGGGCGCGCGCCAGGCGGGGCGGGGCGG
GGCGAGGGGCGGGGCGGGGCGAGGCGGAGAGGTGCGGCGGCAGCCAATCAGAGCGGCGCGCTCCGAAAGTTTCCT
TTTATGGCGAGGCGGCGGCGGCGGCGGCCCTATAAAAAGCGAAGCGCGCGGCGGGCGGGAGTCGCTGCGCGCTGC
CTTCGCCCCGTGCCCCGCTCCGCCGCCGCCTCGCGCCGCCCGCCCCGGCTCTGACTGACCGCGTTACTCCCACAG
GTGAGCGGGCGGGACGGCCCTTCTCCTCCGGGCTGTAATTAGCGCTTGGTTTAATGACGGCTTGTTTCTTTTCTG
TGGCTGCGTGAAAGCCTTGAGGGGCTCCGGGAGGGCCCTTTGTGCGGGGGGAGCGGCTCGGGGCTGTCCGCGGGG
GGACGGCTGCCTTCGGGGGGGACGGGGCAGGGCGGGGTTCGGCTTCTGGCGTGTGACCGGCGGCTCTAGAGCCTC
TGCTAACCATGTTCATGCCTTCTTCTTTTTCCTACAGCTCCTGGGCAACGTGCTGGTTATTGTGCTGTCTCATCA
TTTTGGCAAAGAATTGGATCCACTAGTGCCACCATGCGCCTCCTGGCCAAGATCATCTGCCTCATGCTGTGGGCC
ATCTGCGTGGCTGAGGACTGCAATGAGCTGCCGCCCAGGAGGAACACAGAGATCCTGACAGGGAGCTGGTCTGAC
CAGACCTACCCTGAGGGCACCCAGGCGATCTACAAGTGCCGGCCGGGCTACAGGAGCCTGGGGAACATCATCATG
GTGTGTAGAAAGGGCGAATGGGTGGCCCTCAACCCCCTGAGGAAGTGCCAGAAGCGGCCCTGTGGCCACCCCGGG
GACACACCCTTCGGGACCTTCACCCTGACCGGCGGCAATGTGTTTGAGTACGGCGTGAAGGCTGTCTACACATGC
AACGAGGGGTACCAGCTGCTGGGCGAGATTAACTACCGGGAGTGTGACACCGATGGGTGGACCAACGACATTCCC
ATCTGTGAGGTGGTCAAGTGTCTCCCCGTGACAGCCCCAGAAAATGGCAAAATCGTGAGCAGCGCCATGGAGCCT
GACCGCGAATATCACTTTGGGCAGGCCGTGAGGTTTGTGTGCAACTCGGGCTACAAAATTGAAGGTGATGAGGAG
ATGCACTGCAGCGATGATGGCTTCTGGTCCAAGGAGAAGCCCAAATGTGTGGAGATCTCCTGCAAGTCTCCCGAC
GTGATCAACGGCAGCCCAATCAGCCAGAAGATTATTTACAAAGAGAACGAGCGCTTCCAGTACAAGTGTAACATG
GGCTATGAGTATTCAGAGAGGGGAGATGCCGTCTGCACTGAGAGCGGCTGGAGACCACTGCCTAGCTGCGAGGAA
AAGAGTTGTGACAACCCTTACATCCCAAATGGCGACTACTCCCCTCTGCGGATCAAACACCGGACCGGGGATGAA
ATCACCTATCAGTGCCGCAATGGATTCTACCCGGCCACCCGCGGCAACACCGCCAAATGCACCAGCACAGGCTGG
ATCCCCGCCCCCCGCTGTACGCTGAAGCCTTGCGACTATCCAGACATCAAGCACGGAGGCCTGTACCACGAAAAC
ATGCGGCGGCCTTATTTCCCTGTGGCAGTGGGGAAGTACTACAGCTACTACTGCGACGAGCACTTCGAGACCCCC
TCTGGCTCCTACTGGGACCACATCCACTGCACACAGGACGGCTGGTCTCCAGCTGTGCCCTGCCTGAGGAAATGC
TACTTCCCCTACCTGGAGAACGGATACAACCAGAACTATGGCCGCAAGTTCGTGCAGGGCAAGAGCATCGATGTG
GCCTGCCACCCTGGCTACGCCCTGCCCAAGGCCCAGACAACTGTGACCTGCATGGAGAATGGTTGGAGCCCCACC
CCGCGCTGCATCCGGGTGTCCTTCACGCTCCGAAGGAAACGAGGAAGCGGAGAAGCCAGACACAAACAGAAAATT
GTGGCACCGGTGAAACAGACTTTGAATTTTGACCTTCTCAAGTTGGCGGGAGACGTCGAGTCCAACCCTGGGCCC
ATGCGCCTGCTGGCCAAGATCATCTGCCTGATGCTGTGGGCCATCTGCGTGGCCAGCGACACCGGCCGCCCCTTC
GTGGAGATGTACAGCGAGATCCCCGAGATCATCCACATGACCGAGGGCCGCGAGCTGGTGATCCCCTGCCGCGTG
ACCAGCCCCAACATCACCGTGACCCTGAAGAAGTTCCCCCTGGACACCCTGATCCCCGACGGCAAGCGCATCATC
TGGGACAGCCGCAAGGGCTTCATCATCAGCAACGCCACCTACAAGGAGATCGGCCTGCTGACCTGCGAGGCCACC
GTGAACGGCCACCTGTACAAGACCAACTACCTGACCCACCGCCAGACCAACACCATCATCGACGTGGTGCTGAGC
CCCAGCCACGGCATCGAGCTGAGCGTGGGCGAGAAGCTGGTGCTGAACTGCACCGCCCGCACCGAGCTGAACGTG
GGCATCGACTTCAACTGGGAGTACCCCAGCAGCAAGCACCAGCACAAGAAGCTGGTGAACCGCGACCTGAAGACC
CAGAGCGGCAGCGAGATGAAGAAGTTCCTGAGCACCCTGACCATCGACGGCGTGACCCGCAGCGACCAGGGCCTG
TACACCTGCGCCGCCAGCAGCGGCCTGATGACCAAGAAGAACAGCACCTTCGTGCGCGTGCACGAGAAGGACAAG
ACCCACACCTGCCCCCCCTGCCCCGCCCCCGAGCTGCTGGGCGGCCCCAGCGTGTTCCTGTTCCCCCCCAAGCCC
AAGGACACCCTGATGATCAGCCGCACCCCCGAGGTGACCTGCGTGGTGGTGGACGTGAGCCACGAGGACCCCGAG
GTGAAGTTCAACTGGTACGTGGACGGCGTGGAGGTGCACAACGCCAAGACCAAGCCCCGCGAGGAGCAGTACAAC
AGCACCTACCGCGTGGTGAGCGTGCTGACCGTGCTGCACCAGGACTGGCTGAACGGCAAGGAGTACAAGTGCAAG
GTGAGCAACAAGGCCCTGCCCGCCCCCATCGAGAAGACCATCAGCAAGGCCAAGGGCCAGCCCCGCGAGCCCCAG
GTGTACACCCTGCCCCCCAGCCGCGACGAGCTGACCAAGAACCAGGTGAGCCTGACCTGCCTGGTGAAGGGCTTC
TACCCCAGCGACATCGCCGTGGAGTGGGAGAGCAACGGCCAGCCCGAGAACAACTACAAGACCACCCCCCCCGTG
CTGGACAGCGACGGCAGCTTCTTCCTGTACAGCAAGCTGACCGTGGACAAGAGCCGCTGGCAGCAGGGCAACGTG
TTCAGCTGCAGCGTGATGCACGAGGCCCTGCACAACCACTACACCCAGAAGAGCCTGAGCCTGAGCCCCGGCTAA
CTCGAGGTGCCTTCTAGTTGCCAGCCATCTGTTGTTTGCCCCTCCCCCGTGCCTTCCTTGACCCTGGAAGGTGCC
ACTCCCACTGTCCTTTCCTAATAAAATGAGGAAATTGCATCGCATTGTCTGAGTAGGTGTCATTCTATTCTGGGG
GGTGGGGTGGGGCAGGACAGCAAGGGGGAGGATTGGGAAGACAATAGCAGGCATGCTGGGGATGCGGTGGGCTCT
ATGGGCGGCCGCAGGAACCCCTAGTGATGGAGTTGGCCACTCCCTCTCTGCGCGCTCGCTCGCTCACTGAGGCCG
GGCGACCAAAGGTCGCCCGACGCCCGGGCTTTGCCCGGGCGGCCTCAGTGAGCGAGCG

In some embodiments, the polynucleotide comprises or consists of the nucleotide sequence of SEQ ID NO: 54, or a nucleotide sequence that has at least 75%, 80%, 85%, 86%, 87%, 88%, 89%, 90%, 91%, 92%, 93%, 94%, 95%, 96%, 97%, 98% or 99% sequence identity thereto.

(SEQ ID NO: 54)
CGCTCGCTCACTGAGGCCGCCCGGGCAAAGCCCGGGCGTCGGGCGACCTTTGGTCGCCCGGCCTCAGTGAGCGAG
CGAGCGCGCAGAGAGGGAGTGGCCAACTCCATCACTAGGGGTTCCTGGCGCGCCCCATTGACGTCAATAATGACG
TATGTTCCCATAGTAACGCCAATAGGGACTTTCCATTGACGTCAATGGGTGGAGTATTTACGGTAAACTGCCCAC
TTGGCAGTACATCAAGTGTATCATATGCCAAGTACGCCCCCTATTGACGTCAATGACGGTAAATGGCCCGCCTGG
CATTATGCCCAGTACATGACCTTATGGGACTTTCCTACTTGGCAGTACATCTACGTATTAGTCATCGCTATTACC
ATGGTCGAGGTGAGCCCCACGTTCTGCTTCACTCTCCCCATCTCCCCCCCCTCCCCACCCCCAATTTTGTATTTA
TTTATTTTTTAATTATTTTGTGCAGCGATGGGGGCGGGGGGGGGGGGGGGGCGCGCGCCAGGCGGGGCGGGGCGG
GGCGAGGGGCGGGGCGGGGCGAGGCGGAGAGGTGCGGCGGCAGCCAATCAGAGCGGCGCGCTCCGAAAGTTTCCT
TTTATGGCGAGGCGGCGGCGGCGGCGGCCCTATAAAAAGCGAAGCGCGCGGCGGGCGGGAGTCGCTGCGCGCTGC
CTTCGCCCCGTGCCCCGCTCCGCCGCCGCCTCGCGCCGCCCGCCCCGGCTCTGACTGACCGCGTTACTCCCACAG
GTGAGCGGGCGGGACGGCCCTTCTCCTCCGGGCTGTAATTAGCGCTTGGTTTAATGACGGCTTGTTTCTTTTCTG
TGGCTGCGTGAAAGCCTTGAGGGGCTCCGGGAGGGCCCTTTGTGCGGGGGGAGCGGCTCGGGGCTGTCCGCGGGG
GGACGGCTGCCTTCGGGGGGGACGGGGCAGGGCGGGGTTCGGCTTCTGGCGTGTGACCGGCGGCTCTAGAGCCTC
TGCTAACCATGTTCATGCCTTCTTCTTTTTCCTACAGCTCCTGGGCAACGTGCTGGTTATTGTGCTGTCTCATCA
TTTTGGCAAAGAATTGGATCCACTAGTGCCACCATGCGCCTGCTGGCCAAGATCATCTGCCTGATGCTGTGGGCC
ATCTGCGTGGCCAGCGACACCGGCCGCCCCTTCGTGGAGATGTACAGCGAGATCCCCGAGATCATCCACATGACC
GAGGGCCGCGAGCTGGTGATCCCCTGCCGCGTGACCAGCCCCAACATCACCGTGACCCTGAAGAAGTTCCCCCTG
GACACCCTGATCCCCGACGGCAAGCGCATCATCTGGGACAGCCGCAAGGGCTTCATCATCAGCAACGCCACCTAC
AAGGAGATCGGCCTGCTGACCTGCGAGGCCACCGTGAACGGCCACCTGTACAAGACCAACTACCTGACCCACCGC
CAGACCAACACCATCATCGACGTGGTGCTGAGCCCCAGCCACGGCATCGAGCTGAGCGTGGGCGAGAAGCTGGTG
CTGAACTGCACCGCCCGCACCGAGCTGAACGTGGGCATCGACTTCAACTGGGAGTACCCCAGCAGCAAGCACCAG
CACAAGAAGCTGGTGAACCGCGACCTGAAGACCCAGAGCGGCAGCGAGATGAAGAAGTTCCTGAGCACCCTGACC
ATCGACGGCGTGACCCGCAGCGACCAGGGCCTGTACACCTGCGCCGCCAGCAGCGGCCTGATGACCAAGAAGAAC
AGCACCTTCGTGCGCGTGCACGAGAAGGACAAGACCCACACCTGCCCCCCCTGCCCCGCCCCCGAGCTGCTGGGC
GGCCCCAGCGTGTTCCTGTTCCCCCCCAAGCCCAAGGACACCCTGATGATCAGCCGCACCCCCGAGGTGACCTGC
GTGGTGGTGGACGTGAGCCACGAGGACCCCGAGGTGAAGTTCAACTGGTACGTGGACGGCGTGGAGGTGCACAAC
GCCAAGACCAAGCCCCGCGAGGAGCAGTACAACAGCACCTACCGCGTGGTGAGCGTGCTGACCGTGCTGCACCAG
GACTGGCTGAACGGCAAGGAGTACAAGTGCAAGGTGAGCAACAAGGCCCTGCCCGCCCCCATCGAGAAGACCATC
AGCAAGGCCAAGGGCCAGCCCCGCGAGCCCCAGGTGTACACCCTGCCCCCCAGCCGCGACGAGCTGACCAAGAAC
CAGGTGAGCCTGACCTGCCTGGTGAAGGGCTTCTACCCCAGCGACATCGCCGTGGAGTGGGAGAGCAACGGCCAG
CCCGAGAACAACTACAAGACCACCCCCCCCGTGCTGGACAGCGACGGCAGCTTCTTCCTGTACAGCAAGCTGACC
GTGGACAAGAGCCGCTGGCAGCAGGGCAACGTGTTCAGCTGCAGCGTGATGCACGAGGCCCTGCACAACCACTAC
ACCCAGAAGAGCCTGAGCCTGAGCCCCGGCTAACGAAGGAAACGAGGAAGCGGAGAAGCCAGACACAAACAGAAA
ATTGTGGCACCGGTGAAACAGACTTTGAATTTTGACCTTCTCAAGTTGGCGGGAGACGTCGAGTCCAACCCTGGG
CCCATGCGCCTCCTGGCCAAGATCATCTGCCTCATGCTGTGGGCCATCTGCGTGGCTGAGGACTGCAATGAGCTG
CCGCCCAGGAGGAACACAGAGATCCTGACAGGGAGCTGGTCTGACCAGACCTACCCTGAGGGCACCCAGGCGATC
TACAAGTGCCGGCCGGGCTACAGGAGCCTGGGGAACATCATCATGGTGTGTAGAAAGGGCGAATGGGTGGCCCTC
AACCCCCTGAGGAAGTGCCAGAAGCGGCCCTGTGGCCACCCCGGGGACACACCCTTCGGGACCTTCACCCTGACC
GGCGGCAATGTGTTTGAGTACGGCGTGAAGGCTGTCTACACATGCAACGAGGGGTACCAGCTGCTGGGCGAGATT
AACTACCGGGAGTGTGACACCGATGGGTGGACCAACGACATTCCCATCTGTGAGGTGGTCAAGTGTCTCCCCGTG
ACAGCCCCAGAAAATGGCAAAATCGTGAGCAGCGCCATGGAGCCTGACCGCGAATATCACTTTGGGCAGGCCGTG
AGGTTTGTGTGCAACTCGGGCTACAAAATTGAAGGTGATGAGGAGATGCACTGCAGCGATGATGGCTTCTGGTCC
AAGGAGAAGCCCAAATGTGTGGAGATCTCCTGCAAGTCTCCCGACGTGATCAACGGCAGCCCAATCAGCCAGAAG
ATTATTTACAAAGAGAACGAGCGCTTCCAGTACAAGTGTAACATGGGCTATGAGTATTCAGAGAGGGGAGATGCC
GTCTGCACTGAGAGCGGCTGGAGACCACTGCCTAGCTGCGAGGAAAAGAGTTGTGACAACCCTTACATCCCAAAT
GGCGACTACTCCCCTCTGCGGATCAAACACCGGACCGGGGATGAAATCACCTATCAGTGCCGCAATGGATTCTAC
CCGGCCACCCGCGGCAACACCGCCAAATGCACCAGCACAGGCTGGATCCCCGCCCCCCGCTGTACGCTGAAGCCT
TGCGACTATCCAGACATCAAGCACGGAGGCCTGTACCACGAAAACATGCGGCGGCCTTATTTCCCTGTGGCAGTG
GGGAAGTACTACAGCTACTACTGCGACGAGCACTTCGAGACCCCCTCTGGCTCCTACTGGGACCACATCCACTGC
ACACAGGACGGCTGGTCTCCAGCTGTGCCCTGCCTGAGGAAATGCTACTTCCCCTACCTGGAGAACGGATACAAC
CAGAACTATGGCCGCAAGTTCGTGCAGGGCAAGAGCATCGATGTGGCCTGCCACCCTGGCTACGCCCTGCCCAAG
GCCCAGACAACTGTGACCTGCATGGAGAATGGTTGGAGCCCCACCCCGCGCTGCATCCGGGTGTCCTTCACGCTC
CTCGAGGTGCCTTCTAGTTGCCAGCCATCTGTTGTTTGCCCCTCCCCCGTGCCTTCCTTGACCCTGGAAGGTGCC
ACTCCCACTGTCCTTTCCTAATAAAATGAGGAAATTGCATCGCATTGTCTGAGTAGGTGTCATTCTATTCTGGGG
GGTGGGGTGGGGCAGGACAGCAAGGGGGAGGATTGGGAAGACAATAGCAGGCATGCTGGGGATGCGGTGGGCTCT
ATGGGCGGCCGCAGGAACCCCTAGTGATGGAGTTGGCCACTCCCTCTCTGCGCGCTCGCTCGCTCACTGAGGCCG
GGCGACCAAAGGTCGCCCGACGCCCGGGCTTTGCCCGGGCGGCCTCAGTGAGCGAGCG

Structure of the Eye

The medicaments disclosed herein may be delivered to a mammalian, preferably human eye in relation to the treatment or prevention of an eye disease, such as age-related macular degeneration (AMD).

The person skilled in the treatment of diseases of the eye will have a detailed and thorough understanding of the structure of the eye. However, the following structures of particular relevance to the invention are described.

Retina

The retina is the multi-layered tissue, which lines the inner posterior chamber of the eye and senses an image of the visual world which is communicated to the brain via the optic nerve. In order from the inside to the outside of the eye, the retina comprises the layers of the neurosensory retina and retinal pigment epithelium, with the choroid lying distal to the retinal pigment epithelium.

Neurosensory Retina and Photoreceptor Cells

The neurosensory retina harbours the photoreceptor cells that directly sense light. It comprises the following layers: internal limiting membrane (ILM); nerve fibre layer; ganglion cell layer; inner plexiform layer; inner nuclear layer; outer plexiform layer; outer nuclear layer (nuclei of the photoreceptors); external limiting membrane (ELM); and inner and outer segments of the rods and cones.

The skilled person will have a detailed understanding of photoreceptor cells. Briefly, photoreceptor cells are specialised neurons located in the retina that convert light into biological signals. Photoreceptor cells comprise rod and cone cells, which are distributed differently across the retina.

Rod cells are distributed mainly across the peripheral parts of the retina. They are highly sensitive and provide for vision at low light levels. There are on average about 125 million rod cells in a normal human retina.

Cone cells are found across the retina, but are particularly highly concentrated in the fovea, a pit in the neurosensory retina that is responsible for central high resolution vision. Cone cells are less sensitive than rod cells and are responsible for colour vision. There are on average about 6-7 million cone cells in a normal human retina.

Retinal Pigment Epithelium

The retinal pigment epithelium (RPE) is a pigmented layer of cells located immediately to the outside of the neurosensory retina, adjacent to the photoreceptor outer segments. The RPE separates the neurosensory retina from the choroidal vasculature and has functions such as recycling the visual pigment from the photoreceptor outer segments. The RPE performs a number of functions, including transport of nutrients and other substances to the photoreceptor cells, and absorption of scattered light to improve vision.

Choroid

The choroid is the vascular layer situated between the RPE and the outer sclera of the eye. The vasculature of the choroid enables provision of oxygen and nutrients to the retina.

Age-Related Macular Degeneration (AMD)

The clinical progression of age-related macular degeneration (AMD) is characterised in stages according to changes in the macula. The hallmark of early AMD is the appearance of drusen, which are accumulations of extracellular debris underneath the retina and appear as yellow spots in the retina during clinical examination and on fundus photographs. Drusen are categorised by size as small (<63 μm), medium (63-124 μm) and large (>124 μm). They are also considered as hard or soft depending on the appearance of their margins on ophthalmological examination. While hard drusen have clearly defined margins, soft drusen have less defined, fluid margins. The Age-related Eye Disease Study (AREDS) fundus photographic severity scale is one of the main classification systems used for this condition.

AMD is classified into “dry” and “wet” (exudative or neovascular) forms. Dry AMD is typically characterised by progressive cell loss in the RPE layer, overlying photoreceptor cells, and frequently also the underlying cells in the choroidal capillary layer. Confluent areas of RPE cell death accompanied by overlying photoreceptor atrophy are referred to as geographic atrophy (GA). Patients with this form of AMD experience a slow and progressive deterioration in central vision.

Wet AMD is characterised by bleeding and/or leakage of fluid from abnormal vessels that have grown from the choroidal vessels (choriocapillaris) beneath the RPE and the macula, which can be responsible for sudden and disabling loss of vision. It has been estimated that much of the vision loss that patients experience is due to such choroidal neovascularisation (CNV) and its secondary complications.

The treatment or prevention of AMD described herein may reduce or prevent the appearance of an AMD phenotype described above. Preferably, the treatment of AMD enables maintenance or improvement in visual function.

In some embodiments, the treatment or prevention of AMD results in a prevention of or reduction in the formation of geographic atrophy. In other embodiments, the treatment or prevention of AMD results in slowing the progression of geographic atrophy. For example, it results in an at least 10%, 20%, 30%, 40%, 50%, 60%, 70%, 80% or 90% reduction in the increase in GA area over the 12 months following administration to a treated eye of a subject, relative to an untreated eye over the same period. In other embodiments, the treatment or prevention of AMD results in the treatment of geographic atrophy, for example a reduction in the amount of geographic atrophy.

In some embodiments, the treatment or prevention of AMD results in a prevention of or reduction in the formation of drusen. In other embodiments, the treatment or prevention of AMD results in a reduction in existing drusen, for example a reduction in the size and/or number of existing drusen.

In some embodiments, the treatment or prevention of AMD results in a prevention of or reduction in complement deposition. In other embodiments, the treatment or prevention of AMD results in a reduction in existing complement deposition.

In some embodiments, the treatment or prevention of AMD results in an improvement in or restoration of vision or visual acuity. In other embodiments, the treatment or prevention of AMD mitigates the loss of vision or visual acuity.

In some embodiments, the treatment or prevention of AMD results in an improvement in or restoration of reading speed in a subject. In other embodiments, the treatment or prevention of AMD mitigates the reduction in reading speed in a subject.

In some embodiments, the treatment or prevention of AMD results in a reduction or prevention of loss of photoreceptors and/or the retinal pigment epithelium (RPE).

Diabetic Retinopathy

Diabetic retinopathy is a condition characterised by damage to the blood vessels of the retina, which is caused by the high blood sugar levels associated with diabetes. If left untreated, diabetic retinopathy can cause blindness.

Although subjects with mild diabetic retinopathy may have good vision, certain types of diabetic retinopathy, namely diabetic macular oedema (DMO) and proliferative diabetic retinopathy (PDR) may threaten the sight of the subject.

Diabetic macular oedema is characterised by the leakage of fluid from the damaged blood vessels in the back of the eye. The leaked fluid accumulates in the macula, which leads to swelling and blurred vision. This can eventually give rise to poor central vision and an inability to read or drive. Side vision usually remains normal.

Proliferative diabetic retinopathy is characterised by the closure of retinal blood vessels, leading to the growth of abnormal, fragile blood vessels on the surface of the retina. This may result in permanent loss of vision due to bleeding into the eye, scarring and retinal detachment. Non-proliferative retinopathy is the early stage of diabetic retinopathy which may lead to proliferative retinopathy if left untreated. Therefore treatments are contemplated to all stages and types of diabetic retinopathy.

Vectors

A vector is a tool that allows or facilitates the transfer of an entity from one environment to another.

Adeno-Associated Viral (AAV) Vectors

In one aspect, the invention provides an AAV vector comprising a polynucleotide of the invention.

Preferably, the AAV vector is in the form of an AAV vector particle.

Methods of preparing and modifying viral vectors and viral vector particles, such as those derived from AAV, are well known in the art.

The AAV vector may comprise an AAV genome or a fragment or derivative thereof.

The polynucleotide comprising nucleotide sequences encoding (i) an anti-VEGF entity and (ii) a negative complement regulator as provided by the present invention may be suitable for packaging in AAV. AAV is known to be capable of packaging genomes of up to 5.2 kb in size (Dong, J.-Y. et al. (1996) Human Gene Therapy 7: 2101-2112).

As such, the nucleotide sequences encoding the anti-VEGF entity and the negative complement regulator may be selected such that the combination is suitable for packaging in AAV. In certain embodiments, functional fragments of an anti-VEGF entity and/or a negative complement regulator (such as those described herein) may be used to ensure that the polynucleotide is suitable for packaging in AAV.

In some embodiments, the polynucleotide is less than or equal to 5.2, 5.1, 5.0, 4.9, 4.8 or 4.7 kb. In preferred embodiments, the present polynucleotide is less than or equal to 4.7 kb.

An AAV genome is a polynucleotide sequence, which may encode functions needed for production of an AAV particle. These functions include those operating in the replication and packaging cycle of AAV in a host cell, including encapsidation of the AAV genome into an AAV particle. Naturally occurring AAVs are replication-deficient and rely on the provision of helper functions in trans for completion of a replication and packaging cycle. Accordingly, the AAV genome of the AAV vector of the invention is typically replication-deficient.

The AAV genome may be in single-stranded form, either positive or negative-sense, or alternatively in double-stranded form.

The AAV genome may be from any naturally derived serotype, isolate or clade of AAV. Thus, the AAV genome may be the full genome of a naturally occurring AAV. As is known to the skilled person, AAVs occurring in nature may be classified according to various biological systems.

Commonly, AAVs are referred to in terms of their serotype. A serotype corresponds to a variant subspecies of AAV which, owing to its profile of expression of capsid surface antigens, has a distinctive reactivity which can be used to distinguish it from other variant subspecies. Typically, a virus having a particular AAV serotype does not efficiently cross-react with neutralising antibodies specific for any other AAV serotype.

AAV serotypes include AAV1, AAV2, AAV3, AAV4, AAV5, AAV6, AAV7, AAV8, AAV9, AAV10 and AAV11, and also recombinant serotypes, such as Rec2 and Rec3, recently identified from primate brain. Any of these AAV serotypes may be used in the invention.

In some embodiments, the AAV vector particle is an AAV1, AAV2, AAV3, AAV4, AAV5, AAV6, AAV7, AAV8, AAV9, AAV10, AAV11, Rec2, Rec3, Rh10, DJ or Anc65 AAV vector particle.

In some embodiments, the AAV vector particle may comprise an AAV1, AAV2, AAV3, AAV4, AAV5, AAV6, AAV7, AAV8, AAV9, AAV10 or AAV11 capsid.

In some embodiments, the AAV may be an AAV1, AAV2, AAV5, AAV7 or AAV8 serotype.

In some embodiments, the AAV may be an AAV2 or AAV8 serotype.

In some embodiments, the AAV may be an AAV2 serotype. In other preferred embodiments, the AAV may be an AAV8 serotype.

The capsid protein may be a mutant capsid protein such as disclosed in WO 2008/124724, which is hereby incorporated by reference.

In some embodiments, the AAV vector comprises an AAV8 capsid with an Y733F mutation.

Reviews of AAV serotypes may be found in Choi et al. (2005) Curr. Gene Ther. 5: 299-310 and Wu et al. (2006) Molecular Therapy 14: 316-27. The sequences of AAV genomes or of elements of AAV genomes including ITR sequences, rep or cap genes for use in the invention may be derived from the following accession numbers for AAV whole genome sequences: Adeno-associated virus 1 NC_002077, AF063497; Adeno-associated virus 2 NC_001401; Adeno-associated virus 3 NC_001729; Adeno-associated virus 3B NC_001863; Adeno-associated virus 4 NC_001829; Adeno-associated virus 5 Y18065, AF085716; Adeno-associated virus 6 NC_001862; Avian AAV ATCC VR-865 AY186198, AY629583, NC_004828; Avian AAV strain DA-1 NC_006263, AY629583; Bovine AAV NC_005889, AY388617.

AAV may also be referred to in terms of clades or clones. This refers to the phylogenetic relationship of naturally derived AAVs, and typically to a phylogenetic group of AAVs which can be traced back to a common ancestor, and includes all descendants thereof. Additionally, AAVs may be referred to in terms of a specific isolate, i.e. a genetic isolate of a specific AAV found in nature. The term genetic isolate describes a population of AAVs which has undergone limited genetic mixing with other naturally occurring AAVs, thereby defining a recognisably distinct population at a genetic level.

The skilled person can select an appropriate serotype, clade, clone or isolate of AAV for use in the invention on the basis of their common general knowledge. For instance, the AAV5 capsid has been shown to transduce primate cone photoreceptors efficiently as evidenced by the successful correction of an inherited colour vision defect (Mancuso et al. (2009) Nature 461: 784-7).

The AAV serotype determines the tissue specificity of infection (or tropism) of an AAV. Accordingly, preferred AAV serotypes for use in AAVs administered to patients in accordance with the invention are those which have natural tropism for or a high efficiency of infection of target cells within the eye. In some embodiments, AAV serotypes for use in the invention are those which transduce cells of the neurosensory retina, retinal pigment epithelium and/or choroid.

Typically, the AAV genome of a naturally derived serotype, isolate or clade of AAV comprises at least one inverted terminal repeat sequence (ITR). An ITR sequence acts in cis to provide a functional origin of replication and allows for integration and excision of the vector from the genome of a cell. In preferred embodiments, one or more ITR sequences flank the nucleotide sequences encoding the anti-VEGF entity and negative complement regulator. The AAV genome typically also comprises packaging genes, such as rep and/or cap genes which encode packaging functions for an AAV particle. The rep gene encodes one or more of the proteins Rep78, Rep68, Rep52 and Rep40 or variants thereof. The cap gene encodes one or more capsid proteins such as VP1, VP2 and VP3 or variants thereof. These proteins make up the capsid of an AAV particle. Capsid variants are discussed below.

A promoter will be operably linked to each of the packaging genes. Specific examples of such promoters include the p5, p19 and p40 promoters (Laughlin et al. (1979) Proc. Natl. Acad. Sci. USA 76: 5567-5571). For example, the p5 and p19 promoters are generally used to express the rep gene, while the p40 promoter is generally used to express the cap gene.

As discussed above, the AAV genome used in the AAV vector of the invention may therefore be the full genome of a naturally occurring AAV. For example, a vectors which between them comprise a full AAV genome may be used to prepare an AAV vector or vector particle in vitro. However, while such a vector may in principle be administered to patients, this will rarely be done in practice. Preferably, the AAV genome will be derivatised for the purpose of administration to patients. Such derivatisation is standard in the art and the invention encompasses the use of any known derivative of an AAV genome, and derivatives which could be generated by applying techniques known in the art. Derivatisation of the AAV genome and of the AAV capsid are reviewed in Coura and Nardi (2007) Virology Journal 4: 99, and in Choi et al. and Wu et al., referenced above.

Derivatives of an AAV genome include any truncated or modified forms of an AAV genome which allow for expression of a transgene from an AAV vector of the invention in vivo. Typically, it is possible to truncate the AAV genome significantly to include minimal viral sequence yet retain the above function. This is preferred for safety reasons to reduce the risk of recombination of the vector with wild-type virus, and also to avoid triggering a cellular immune response by the presence of viral gene proteins in the target cell.

Typically, a derivative will include at least one inverted terminal repeat sequence (ITR), preferably more than one ITR, such as two ITRs or more. One or more of the ITRs may be derived from AAV genomes having different serotypes, or may be a chimeric or mutant ITR. A preferred mutant ITR is one having a deletion of a trs (terminal resolution site). This deletion allows for continued replication of the genome to generate a single-stranded genome which contains both coding and complementary sequences, i.e. a self-complementary AAV genome. This allows for bypass of DNA replication in the target cell, and so enables accelerated transgene expression.

The one or more ITRs will preferably flank the nucleotide sequence encoding the anti-VEGF entity and negative complement regulator at either end. The inclusion of one or more ITRs is preferred to aid concatamer formation of the vector of the invention in the nucleus of a host cell, for example following the conversion of single-stranded vector DNA into double-stranded DNA by the action of host cell DNA polymerases. The formation of such episomal concatamers protects the vector construct during the life of the host cell, thereby allowing for prolonged expression of the transgene in vivo.

In preferred embodiments, ITR elements will be the only sequences retained from the native AAV genome in the derivative. Thus, a derivative will preferably not include the rep and/or cap genes of the native genome and any other sequences of the native genome. This is preferred for the reasons described above, and also to reduce the possibility of integration of the vector into the host cell genome. Additionally, reducing the size of the AAV genome allows for increased flexibility in incorporating other sequence elements (such as regulatory elements) within the vector in addition to the transgene.

The following portions could therefore be removed in a derivative of the invention: the replication (rep) and capsid (cap) genes. However, in some embodiments, derivatives may additionally include one or more rep and/or cap genes or other viral sequences of an AAV genome. Naturally occurring AAV integrates with a high frequency at a specific site on human chromosome 19, and shows a negligible frequency of random integration, such that retention of an integrative capacity in the vector may be tolerated in a therapeutic setting.

Where a derivative comprises capsid proteins i.e. VP1, VP2 and/or VP3, the derivative may be a chimeric, shuffled or capsid-modified derivative of one or more naturally occurring AAVs. In particular, the invention encompasses the provision of capsid protein sequences from different serotypes, clades, clones, or isolates of AAV within the same vector (i.e. a pseudotyped vector).

Chimeric, shuffled or capsid-modified derivatives will be typically selected to provide one or more desired functionalities for the AAV vector. Thus, these derivatives may display increased efficiency of gene delivery, decreased immunogenicity (humoral or cellular), an altered tropism range and/or improved targeting of a particular cell type compared to an AAV vector comprising a naturally occurring AAV genome, such as that of AAV2 or AAV8. Increased efficiency of gene delivery may be effected by improved receptor or co-receptor binding at the cell surface, improved internalisation, improved trafficking within the cell and into the nucleus, improved uncoating of the viral particle and improved conversion of a single-stranded genome to double-stranded form. Increased efficiency may also relate to an altered tropism range or targeting of a specific cell population, such that the vector dose is not diluted by administration to tissues where it is not needed.

Chimeric capsid proteins include those generated by recombination between two or more capsid coding sequences of naturally occurring AAV serotypes. This may be performed for example by a marker rescue approach in which non-infectious capsid sequences of one serotype are co-transfected with capsid sequences of a different serotype, and directed selection is used to select for capsid sequences having desired properties. The capsid sequences of the different serotypes can be altered by homologous recombination within the cell to produce novel chimeric capsid proteins.

Chimeric capsid proteins also include those generated by engineering of capsid protein sequences to transfer specific capsid protein domains, surface loops or specific amino acid residues between two or more capsid proteins, for example between two or more capsid proteins of different serotypes.

Shuffled or chimeric capsid proteins may also be generated by DNA shuffling or by error-prone PCR. Hybrid AAV capsid genes can be created by randomly fragmenting the sequences of related AAV genes e.g. those encoding capsid proteins of multiple different serotypes and then subsequently reassembling the fragments in a self-priming polymerase reaction, which may also cause crossovers in regions of sequence homology. A library of hybrid AAV genes created in this way by shuffling the capsid genes of several serotypes can be screened to identify viral clones having a desired functionality. Similarly, error prone PCR may be used to randomly mutate AAV capsid genes to create a diverse library of variants which may then be selected for a desired property.

The sequences of the capsid genes may also be genetically modified to introduce specific deletions, substitutions or insertions with respect to the native wild-type sequence. In particular, capsid genes may be modified by the insertion of a sequence of an unrelated protein or peptide within an open reading frame of a capsid coding sequence, or at the N- and/or C-terminus of a capsid coding sequence. This may be done, for example, to confer or change tropism towards specific cell receptors.

The unrelated protein or peptide may advantageously be one which acts as a ligand for a particular cell type, thereby conferring improved binding to a target cell or improving the specificity of targeting of the vector to a particular cell population. An example might include the use of RGD peptide to block uptake in the retinal pigment epithelium and thereby enhance transduction of surrounding retinal tissues (Cronin et al. (2008) ARVO Abstract: D1048). The unrelated protein may also be one which assists purification of the viral particle as part of the production process, i.e. an epitope or affinity tag. The site of insertion will typically be selected so as not to interfere with other functions of the viral particle e.g. internalisation, trafficking of the viral particle. The skilled person can identify suitable sites for insertion based on their common general knowledge. Particular sites are disclosed in Choi et al., referenced above and Dalkara et al. (Science, Translational Medicine, 2013; 5(189)).

The invention additionally encompasses the provision of sequences of an AAV genome in a different order and configuration to that of a native AAV genome. The invention also encompasses the replacement of one or more AAV sequences or genes with sequences from another virus or with chimeric genes composed of sequences from more than one virus. Such chimeric genes may be composed of sequences from two or more related viral proteins of different viral species.

The AAV vector of the invention may take the form of a nucleotide sequence comprising an AAV genome or derivative thereof and a sequence encoding the anti-VEGF entity and negative complement regulator transgenes or derivatives thereof.

The AAV particles of the invention include transcapsidated forms wherein an AAV genome or derivative having an ITR of one serotype is packaged in the capsid of a different serotype.

The AAV particles of the invention also include mosaic forms wherein a mixture of unmodified capsid proteins from two or more different serotypes makes up the viral capsid.

The AAV particle also includes chemically modified forms bearing ligands adsorbed to the capsid surface. For example, such ligands may include antibodies for targeting a particular cell surface receptor.

Thus, for example, the AAV particles of the invention include those with an AAV2 genome and AAV2 capsid proteins (AAV2/2), those with an AAV2 genome and AAV5 capsid proteins (AAV2/5) and those with an AAV2 genome and AAV8 capsid proteins (AAV2/8), as well as those with an AAV2 genome and capsid proteins of more than one serotype.

The AAV vector may comprise multiple copies (e.g., 2, 3 etc.) of the nucleotide sequence referred to herein.

In some embodiments, the polynucleotide further comprises one or more AAV ITRs. In preferred embodiments, the polynucleotide further comprises two AAV ITRs. In some embodiments, the polynucleotide comprises an AAV ITR at its 5′ end and an AAV ITR at its 3′ end. In some embodiments, the AAV ITRs are AAV2 or AAV8 ITRs. In preferred embodiments, the AAV ITRs are AAV2 ITRs.

In some embodiments, the polynucleotide comprises a 5′ AAV ITR with the nucleotide sequence of SEQ ID NO: 55, or a nucleotide sequence that has at least 75%, 80%, 85%, 86%, 87%, 88%, 89%, 90%, 91%, 92%, 93%, 94%, 95%, 96%, 97%, 98% or 99% sequence identity thereto.

SEQ ID NO: 55
CGCTCGCTCACTGAGGCCGCCCGGGCAAAGCCCGGGCGTCGGGCGACCT
TTGGTCGCCCGGCCTCAGTGAGCGAGCGAGCGCGCAGAGAGGGAGTGGC
CAACTCCATCACTAGGGGTTCCT

In some embodiments, the polynucleotide further comprises the nucleotide sequence of SEQ ID NO: 56, or a nucleotide sequence that has at least 75%, 80%, 85%, 86%, 87%, 88%, 89%, 90%, 91%, 92%, 93%, 94%, 95%, 96%, 97%, 98% or 99% sequence identity thereto, immediately adjacent to the 3′ end of the 5′ ITR.

SEQ ID NO: 56
TGTAGTTAATGATTAACCCGCCATGCTACTTATCTACGTAGCCATGCTC
TAGGTACC

In some embodiments, the polynucleotide comprises a 5′ AAV ITR with the nucleotide sequence of SEQ ID NO: 65, or a nucleotide sequence that has at least 75%, 80%, 85%, 86%, 87%, 88%, 89%, 90%, 91%, 92%, 93%, 94%, 95%, 96%, 97%, 98% or 99% sequence identity thereto.

SEQ ID NO: 65
aggaacccctagtgatggagttggccactccctctctgcgcgctcgctc
gctcactgaggccgcccgggcaaagcccgggcgtcgggcgacctttggt
cgcccggcctcagtgagcgagcgagcgcgcagagagggagtggccaa

In some embodiments, the polynucleotide comprises a 3′ AAV ITR with the nucleotide sequence of SEQ ID NO: 57, or a nucleotide sequence that has at least 75%, 80%, 85%, 86%, 87%, 88%, 89%, 90%, 91%, 92%, 93%, 94%, 95%, 96%, 97%, 98% or 99% sequence identity thereto.

SEQ ID NO: 57
AGGAACCCCTAGTGATGGAGTTGGCCACTCCCTCTCTGCGCGCTCGCTC
GCTCACTGAGGCCGGGCGACCAAAGGTCGCCCGACGCCCGGGCTTTGCC
CGGGCGGCCTCAGTGAGCGAGCG

In some embodiments, the polynucleotide further comprises the nucleotide sequence of SEQ ID NO: 58, or a nucleotide sequence that has at least 75%, 80%, 85%, 86%, 87%, 88%, 89%, 90%, 91%, 92%, 93%, 94%, 95%, 96%, 97%, 98% or 99% sequence identity thereto, immediately adjacent to the 5′ end of the 3′ ITR.

SEQ ID NO: 58
CTTCTGAGGCGGAAAGAACCAGCTGGGGCTCGACTAGAGCATGGCTACG
TAGATAAGTAGCATGGCGGGTTAATCATTAACTACA

In some embodiments, the polynucleotide comprises a 3′ AAV ITR with the nucleotide sequence of SEQ ID NO: 66, or a nucleotide sequence that has at least 75%, 80%, 85%, 86%, 87%, 88%, 89%, 90%, 91%, 92%, 93%, 94%, 95%, 96%, 97%, 98% or 99% sequence identity thereto.

SEQ ID NO: 66
aggaacccctagtgatggagttggccactccctctctgcgcgctcgctc
gctcactgaggccgggcgaccaaaggtcgcccgacgcccgggctttgcc
cgggcggcctcagtgagcgagcgagcgcgcagagagggagtggccaa

Promoters and Regulatory Sequences

The polynucleotide or vector of the invention may also include elements allowing for the expression of the anti-VEGF entity and/or negative complement regulator transgenes in vitro or in vivo. These may be referred to as expression control sequences. Thus, the polynucleotide or vector typically comprises expression control sequences (e.g. comprising a promoter sequence) operably linked to the nucleotide sequence encoding the transgene.

In some embodiments, the polynucleotide or vector comprises nucleotide sequences encoding: (a) a CMV promoter or CAG promoter, optionally wherein the CMV or CAG promoter is upstream of the nucleotide sequences encoding the anti-VEGF entity and/or the negative complement regulator; (b) a WPRE regulatory element, optionally wherein the WPRE regulatory element is downstream of the nucleotide sequences encoding the anti-VEGF entity and/or the negative complement regulator; and/or (c) a poly-A signal, such as a Bovine Growth Hormone poly-A signal, optionally wherein the poly-A signal is downstream of the nucleotide sequences encoding the anti-VEGF entity and/or the negative complement regulator.

In some embodiments, the polynucleotide or vector comprises:
(a) a 5′ AAV ITR;
(b) a promoter, optionally a CMV promoter;
(c) a nucleotide sequence encoding a negative complement regulator;
(d) a linker, optionally wherein the linker comprises or is defined by a Furin cleavage site, GSG, 11a1D and an F2A sequence;
(e) a nucleotide sequence encoding an anti-VEGF entity, preferably aflibercept;
(f) optionally, a WPRE regulatory element, optionally a WPRE3 regulatory element;
(g) a poly-A signal, optionally a Bovine Growth Hormone poly-A signal; and
(h) a 3′ AAV ITR.

The negative complement regulator may be selected from CFI and FHL-1.

Elements (c) and (e) may be in the reciprocal positions.

As such, in some embodiments, the polynucleotide or vector comprises:
(a) a 5′ AAV ITR;
(b) a promoter, optionally a CMV promoter;
(c) a nucleotide sequence encoding an anti-VEGF entity, preferably aflibercept;
(d) a linker, optionally wherein the linker comprises or is defined by a Furin cleavage site, GSG, 11a1D and an F2A sequence;
(e) a nucleotide sequence encoding a negative complement regulator;
(f) optionally, a WPRE regulatory element, optionally a WPRE3 regulatory element;
(g) a poly-A signal, optionally a Bovine Growth Hormone poly-A signal; and
(h) a 3′ AAV ITR.

In some embodiments, the polynucleotide or vector comprises:
(a) a 5′ AAV ITR;
(b) a promoter, optionally a CAG promoter;
(c) a nucleotide sequence encoding a negative complement regulator, preferably FHL-1;
(d) a linker, optionally wherein the linker comprises or is defined by a Furin cleavage site, GSG, 11a1D and an F2A sequence;
(e) a nucleotide sequence encoding an anti-VEGF entity, preferably aflibercept;
(f) optionally, a WPRE regulatory element, further optionally a WPRE3 regulatory element;
(g) a poly-A signal, optionally a Bovine Growth Hormone poly-A signal; and
(h) a 3′ AAV ITR.

Elements (c) and (e) may be in the reciprocal positions.

As such, in some embodiments, the polynucleotide or vector comprises:
(a) a 5′ AAV ITR;
(b) a promoter, optionally a CAG promoter;
(c) a nucleotide sequence encoding an anti-VEGF entity, preferably aflibercept;
(d) a linker, optionally wherein the linker comprises or is defined by a Furin cleavage site, GSG, 11a1D and an F2A sequence;
(e) a nucleotide sequence encoding a negative complement regulator, preferably FHL-1;
(f) optionally, a WPRE regulatory element, further optionally a WPRE3 regulatory element;
(g) a poly-A signal, optionally a Bovine Growth Hormone poly-A signal; and
(h) a 3′ AAV ITR.

The polynucleotide may comprise (a)-(h) in order, from 5′ to 3′. Suitably, (a) is at the 5′ end of the polynucleotide and (h) is at the 3′ end of the polynucleotide. Suitably, (b) is upstream of (c)-(g). Suitably, (f) is downstream of (b)-(e). Suitably, (g) is downstream of (b)-(f). Suitably, (c) is upstream of (d) and (e) is downstream of (d).

Any suitable promoter may be used, the selection of which may be readily made by the skilled person. The promoter sequence may be constitutively active (i.e. operational in any host cell background), or alternatively may be active only in a specific host cell environment, thus allowing for targeted expression of the transgene in a particular cell type (e.g. a tissue-specific promoter). The promoter may show inducible expression in response to presence of another factor, for example a factor present in a host cell. In any event, where the vector is administered for therapy, it is preferred that the promoter should be functional in the target cell background.

In some embodiments, it is preferred that the promoter shows retinal-cell specific expression in order to allow for the transgene to only be expressed in retinal cell populations. Thus, expression from the promoter may be retinal-cell specific, for example confined only to cells of the neurosensory retina and retinal pigment epithelium. The promoter may also enable expression, in particular tissue- or cell-specific expression, in ganglion cells, Mueller cells, photoreceptor cells, retinal pigment epithelium cells, and/or endothelial cells.

Preferred promoters, which are not retinal-cell specific, include the chicken beta-actin (CBA) promoter, optionally in combination with a cytomegalovirus (CMV) enhancer element. An example promoter for use in the invention is a CAG promoter, for example the promoter used in the rAVE expression cassette (GeneDetect.com).

In preferred embodiments, the polynucleotide or vector comprises a CMV promoter.

An example CMV promoter sequence is:

(SEQ ID NO: 59)
GGAGTTCCGCGTTACATAACTTACGGTAAATGGCCCGCCTGGCTGACCGCCCAACGACCCCCGCCCATTGAC
GTCAATAATGACGTATGTTCCCATAGTAACGCCAATAGGGACTTTCCATTGACGTCAATGGGTGGAGTATTT
ACGGTAAACTGCCCACTTGGCAGTACATCAAGTGTATCATATGCCAAGTACGCCCCCTATTGACGTCAATGA
CGGTAAATGGCCCGCCTGGCATTATGCCCAGTACATGACCTTATGGGACTTTCCTACTTGGCAGTACATCTA
CGTATTAGTCATCGCTATTACCATGGTGATGCGGTTTTGGCAGTACATCAATGGGCGTGGATAGCGGTTTGA
CTCACGGGGATTTCCAAGTCTCCACCCCATTGACGTCAATGGGAGTTTGTTTTGGCACCAAAATCAACGGGA
CTTTCCAAAATGTCGTAACAACTCCGCCCCATTGACGCAAATGGGCGGTAGGCGTGTACGGTGGGAGGTCTA
TATAAGCAGAGCTCGTTTAGTGAACCGTCAGATCGCCTGGAGACGCCATCCACGCTGTTTTGACCTCCATAG
AAGACACCG

In some embodiments, the polynucleotide or vector comprises a promoter with a nucleotide sequence that has at least 75%, 80%, 85% 90%, 95%, 96%, 97%, 98% or 99% identity to SEQ ID NO: 59. Preferably, wherein the nucleotide sequence substantially retains the functional activity of the promoter represented by SEQ ID NO: 59.

In other embodiments, the polynucleotide or vector comprises a promoter with the nucleotide sequence of SEQ ID NO: 60.

An example CAG promoter sequence is:

(SEQ ID NO: 60)
CCATTGACGTCAATAATGACGTATGTTCCCATAGTAACGCCAATAGGGACTTTCCATTGACGTCAATGGGTG
GAGTATTTACGGTAAACTGCCCACTTGGCAGTACATCAAGTGTATCATATGCCAAGTACGCCCCCTATTGAC
GTCAATGACGGTAAATGGCCCGCCTGGCATTATGCCCAGTACATGACCTTATGGGACTTTCCTACTTGGCAG
TACATCTACGTATTAGTCATCGCTATTACCATGGTCGAGGTGAGCCCCACGTTCTGCTTCACTCTCCCCATC
TCCCCCCCCTCCCCACCCCCAATTTTGTATTTATTTATTTTTTAATTATTTTGTGCAGCGATGGGGGCGGGG
GGGGGGGGGGGGCGCGCGCCAGGCGGGGCGGGGCGGGGCGAGGGGCGGGGCGGGGCGAGGCGGAGAGGTGCG
GCGGCAGCCAATCAGAGCGGCGCGCTCCGAAAGTTTCCTTTTATGGCGAGGCGGCGGCGGCGGCGGCCCTAT
AAAAAGCGAAGCGCGCGGCGGGCGGGAGTCGCTGCGCGCTGCCTTCGCCCCGTGCCCCGCTCCGCCGCCGCC
TCGCGCCGCCCGCCCCGGCTCTGACTGACCGCGTTACTCCCACAGGTGAGCGGGCGGGACGGCCCTTCTCCT
CCGGGCTGTAATTAGCGCTTGGTTTAATGACGGCTTGTTTCTTTTCTGTGGCTGCGTGAAAGCCTTGAGGGG
CTCCGGGAGGGCCCTTTGTGCGGGGGGAGCGGCTCGGGGCTGTCCGCGGGGGGACGGCTGCCTTCGGGGGGG
ACGGGGCAGGGCGGGGTTCGGCTTCTGGCGTGTGACCGGCGGCTCTAGAGCCTCTGCTAACCATGTTCATGC
CTTCTTCTTTTTCCTACAGCTCCTGGGCAACGTGCTGGTTATTGTGCTGTCTCATCATTTTGGCAAAGAATT
GGATCC

In some embodiments, the polynucleotide or vector comprises a promoter with a nucleotide sequence that has at least 75%, 80%, 85% 90%, 95%, 96%, 97%, 98% or 99% identity to SEQ ID NO: 60. Preferably, wherein the nucleotide sequence substantially retains the functional activity of the promoter represented by SEQ ID NO: 60.

In other embodiments, the polynucleotide or vector comprises a promoter with the nucleotide sequence of SEQ ID NO: 60.

Examples of promoters based on human sequences that would induce retina-specific gene expression include rhodopsin kinase for rods and cones (Allocca et al. (2007) J. Virol. 81: 11372-80), PR2.1 for cones only (Mancuso et al. (2009) Nature 461: 784-7) and/or RPE65 (Bainbridge et al. (2008) N. Engl. J. Med. 358: 2231-9) or VMD2 (Esumi et al. (2004) J. Biol. Chem. 279: 19064-73) for the retinal pigment epithelium.

The polynucleotide or vector of the invention may also comprise one or more additional regulatory sequences which may act pre- or post-transcriptionally. The regulatory sequence may be part of the native transgene locus or may be a heterologous regulatory sequence. The polynucleotide or vector of the invention may comprise portions of the 5′-UTR or 3′-UTR from the native transgene transcript.

Regulatory sequences are any sequences which facilitate expression of the transgene, i.e. act to increase expression of a transcript, improve nuclear export of mRNA or enhance its stability. Such regulatory sequences include for example enhancer elements, post-transcriptional regulatory elements and polyadenylation sites.

A preferred polyadenylation site is the Bovine Growth Hormone poly-A (bGH poly-A) signal.

An example Bovine Growth Hormone poly-A (bGH poly-A) signal is:

(SEQ ID NO: 61)
GTGCCTTCTAGTTGCCAGCCATCTGTTGTTTGCCCCTCCCCCGTGCCTTCCTTGACCCTGGAAGGTGCCACT
CCCACTGTCCTTTCCTAATAAAATGAGGAAATTGCATCGCATTGTCTGAGTAGGTGTCATTCTATTCTGGGG
GGTGGGGTGGGGCAGGACAGCAAGGGGGAGGATTGGGAAGACAATAGCAGGCATGCTGGGGATGCGGTGGGC
TCTATGG

A further example Bovine Growth Hormone poly-A (bGH poly-A) signal is:

(SEQ ID NO: 62)
TCGCTGATCAGCCTCGACTGTGCCTTCTAGTTGCCAGCCATCTGTTGTTTGCCCCTCCCCCGTGCCTTCCTT
GACCCTGGAAGGTGCCACTCCCACTGTCCTTTCCTAATAAAATGAGGAAATTGCATCGCATTGTCTGAGTAG
GTGTCATTCTATTCTGGGGGGTGGGGTGGGGCAGGACAGCAAGGGGGAGGATTGGGAAGACAATAGCAGGCA
TGCTGGGGATGCGGTGGGCTCTATGGCTTCTGAGGCGGAAAGAACCAGCTGGGG

In some embodiments, the polynucleotide or vector comprises a polyadenylation signal with a nucleotide sequence that has at least 75%, 80%, 85%, 90%, 95%, 96%, 97%, 98% or 99% identity to SEQ ID NO: 61 or 62. Preferably, wherein the nucleotide sequence substantially retains the functional activity of the polyadenylation signal represented by SEQ ID NO: 61 or 62.

In other embodiments, the polynucleotide or vector comprises a polyadenylation signal with the nucleotide sequence of SEQ ID NO: 61 or 62.

In the context of the polynucleotide or vector of the invention, such regulatory sequences will be cis-acting. However, the invention also encompasses the use of trans-acting regulatory sequences located on additional genetic constructs.

A preferred post-transcriptional regulatory element for use in an AAV vector of the invention is the woodchuck hepatitis post-transcriptional regulatory element (WPRE) or a variant thereof.

An example WPRE is:

(SEQ ID NO: 63)
ATCAACCTCTGGATTACAAAATTTGTGAAAGATTGACTGGTATTCTTAACTATGTTGCTCCTTTTACGCTAT
GTGGATACGCTGCTTTAATGCCTTTGTATCATGCTATTGCTTCCCGTATGGCTTTCATTTTCTCCTCCTTGT
ATAAATCCTGGTTGCTGTCTCTTTATGAGGAGTTGTGGCCCGTTGTCAGGCAACGTGGCGTGGTGTGCACTG
TGTTTGCTGACGCAACCCCCACTGGTTGGGGCATTGCCACCACCTGTCAGCTCCTTTCCGGGACTTTCGCTT
TCCCCCTCCCTATTGCCACGGCGGAACTCATCGCCGCCTGCCTTGCCCGCTGCTGGACAGGGGCTCGGCTGT
TGGGCACTGACAATTCCGTGGTGTTGTCGGGGAAATCATCGTCCTTTCCTTGGCTGCTCGCCTGTGTTGCCA
CCTGGATTCTGCGCGGGACGTCCTTCTGCTACGTCCCTTCGGCCCTCAATCCAGCGGACCTTCCTTCCCGCG
GCCTGCTGCCGGCTCTGCGGCCTCTTCCGCGTCTTCGCCTTCGCCCTCAGACGAGTCGGATCTCCCTTTGGG
CCGCCTCCCCGC

WPRE is a tripartite element containing gamma, alpha and beta elements, in the given order. A shortened version of WPRE, which contains only minimal gamma and alpha elements (referred to as WPRE3; Choi, J.-H. et al. (2014) Molecular Brain 7: 17), may also be used in the invention.

An example WPRE3 sequence is:

(SEQ ID NO: 64)
AATCAACCTCTGGATTACAAAATTTGTGAAAGATTGACTGGTATTCTTAACTATGTTGCTCCTTTTACGCTA
TGTGGATACGCTGCTTTAATGCCTTTGTATCATGCTATTGCTTCCCGTATGGCTTTCATTTTCTCCTCCTTG
TATAAATCCTGGTTAGTTCTTGCCACGGCGGAACTCATCGCCGCCTGCCTTGCCCGCTGCTGGACAGGGGCT
CGGCTGTTGGGCACTGACAATTCCGTGGT

In some embodiments, the polynucleotide or vector comprises a post-transcriptional regulatory element with a nucleotide sequence that has at least 75%, 80%, 85%, 90%, 95%, 96%, 97%, 98% or 99% identity to SEQ ID NO: 63 or 64. Preferably, wherein the nucleotide sequence substantially retains the functional activity of the post-transcriptional regulatory element represented by SEQ ID NO: 63 or 64.

The WPRE may be derived from wild type or modified woodchuck hepatitis virus posttranscriptional regulatory element (WPRE) sequences

In other embodiments, the polynucleotide or vector comprises a post-transcriptional regulatory element with the nucleotide sequence of SEQ ID NO: 63 or 64.

Another regulatory sequence which may be used in a polynucleotide or vector of the invention is a scaffold-attachment region (SAR). Additional regulatory sequences may be readily selected by the skilled person.

Method of Administration

The products, polynucleotide or vector of the invention may be administered systemically (for example by peripheral vein infusion) and may be administered locally or regionally (for example to the CNS system by intrathecal injection). In preferred embodiments, the product, polynucleotide or vector is administered intraocularly.

The term “intraocular” refers to the interior of the eye, thus intraocular administration relates to the administration to the interior of the eye of a subject

In some embodiments, the product, polynucleotide or vector is administered to the eye of a subject by subretinal, direct retinal, suprachoroidal or intravitreal injection. In some embodiments, said administration is performed by a robot.

The volume of the medicament composition injected may, for example, be about 10-500 μL, for example about 50-500, 100-500, 200-500, 300-500, 400-500, 50-250, 100-250, 200-250 or 50-150 μL. The volume may, for example, be about 10, 50, 100, 150, 200, 250, 300, 350, 400, 450 or 500 μL. Preferably, the volume of the medicament composition injected is 100 μL.

The skilled person will be familiar with and well able to carry out individual subretinal, direct retinal, suprachoroidal or intravitreal injections.

Preferably, the product, polynucleotide or vector is administered by subretinal injection.

In some embodiments, the product, polynucleotide, vector or pharmaceutical composition comprising the same is administered not more than once, or not more than twice, during the lifetime of a subject.

Subretinal Injection

Subretinal injections are injections into the subretinal space, i.e. underneath the neurosensory retina. During a subretinal injection, the injected material is directed into, and creates a space between, the photoreceptor cell and retinal pigment epithelial (RPE) layers.

When the injection is carried out through a small retinotomy, a retinal detachment may be created. The detached, raised layer of the retina that is generated by the injected material is referred to as a “bleb”.

The hole created by the subretinal injection must be sufficiently small that the injected solution does not significantly reflux back into the vitreous cavity after administration. Such refluxwould be particularly problematic when a medicament is injected, because the effects of the medicament would be directed away from the target zone. Preferably, the injection creates a self-sealing entry point in the neurosensory retina, i.e. once the injection needle is removed, the hole created by the needle reseals such that very little or substantially no injected material is released through the hole.

To facilitate this process, specialist subretinal injection needles are commercially available (e.g. DORC 41G Teflon subretinal injection needle, Dutch Ophthalmic Research Center International BV, Zuidland, The Netherlands). These are needles designed to carry out subretinal injections.

Unless damage to the retina occurs during the injection, and as long as a sufficiently small needle is used, substantially all injected material remains localised between the detached neurosensory retina and the RPE at the site of the localised retinal detachment (i.e. does not reflux into the vitreous cavity). Indeed, the typical persistence of the bleb over a short time frame indicates that there is usually little escape of the injected material into the vitreous. The bleb may dissipate over a longer time frame as the injected material is absorbed.

Visualisations of the eye, in particular the retina, for example using optical coherence tomography, may be made pre-operatively.

The volume of the medicament composition injected may, for example, be about 10-500 μL, for example about 50-500, 100-500, 200-500, 300-500, 400-500, 50-250, 100-250, 200-250 or 50-150 μL. The volume may, for example, be about 10, 50, 100, 150, 200, 250, 300, 350, 400, 450 or 500 μL. Preferably, the volume of the medicament composition injected is 100 μL. Larger volumes may increase the risk of stretching the retina, while smaller volumes may be difficult to see.

Two-Step Subretinal Injection

The product, polynucleotide or vector of the invention may be delivered with increased accuracy and safety by using a two-step method in which a localised retinal detachment is created by the subretinal injection of a first solution. The first solution does not comprise the product, polynucleotide or vector. A second subretinal injection is then used to deliver the medicament comprising the product, polynucleotide or vector into the subretinal fluid of the bleb created by the first subretinal injection. Because the injection delivering the medicament is not being used to detach the retina, a specific volume of solution may be injected in this second step.

In some embodiments, the subretinal injection of the vector comprises the steps:
(a) administering a solution to the subject by subretinal injection in an amount effective to at least partially detach the retina to form a subretinal bleb, wherein the solution does not comprise the product, polynucleotide or vector; and
(b) administering a medicament composition by subretinal injection into the bleb formed by step (a), wherein the medicament comprises the product, polynucleotide or vector.

The volume of solution injected in step (a) to at least partially detach the retina may be, for example, about 10-1000 μL, for example about 50-1000, 100-1000, 250-1000, 500-1000, 10-500, 50-500, 100-500, 250-500 μL. The volume may be, for example, about 10, 50, 100, 200, 300, 400, 500, 600, 700, 800, 900 or 1000 μL.

The volume of the medicament composition injected in step (b) may be, for example, about 10-500 μL, for example about 50-500, 100-500, 200-500, 300-500, 400-500, 50-250, 100-250, 200-250 or 50-150 μL. The volume may be, for example, about 10, 50, 100, 150, 200, 250, 300, 350, 400, 450 or 500 μL. Preferably, the volume of the medicament composition injected in step (b) is 100 μL. Larger volumes may increase the risk of stretching the retina, while smaller volumes may be difficult to see.

The solution that does not comprise the medicament (i.e. the “solution” of step (a)) may be similarly formulated to the solution that does comprise the medicament, as described below.

A preferred solution that does not comprise the medicament is balanced saline solution (BSS) or a similar buffer solution matched to the pH and osmolality of the subretinal space.

Visualising the Retina During Surgery

Under certain circumstances, for example during end-stage retinal degenerations, identifying the retina is difficult because it is thin, transparent and difficult to see against the disrupted and heavily pigmented epithelium on which it sits. The use of a blue vital dye (e.g. Brilliant Peel®, Geuder; MembraneBlue-Dual®, Dorc) may facilitate the identification of the retinal hole made for the retinal detachment procedure (i.e. step (a) in the two-step subretinal injection method of the invention) so that the medicament can be administered through the same hole without the risk of reflux back into the vitreous cavity.

The use of the blue vital dye also identifies any regions of the retina where there is a thickened internal limiting membrane or epiretinal membrane, as injection through either of these structures would hinder clean access into the subretinal space. Furthermore, contraction of either of these structures in the immediate post-operative period could lead to stretching of the retinal entry hole, which could lead to reflux of the medicament into the vitreous cavity.

Suprachoroidal Injection

The product, polynucleotide or vector of the invention may be delivered to the suprachoroidal space using an ab externo approach that utilises an microcatheter (see, for example, Peden et al. (2011) PLoS One 6(2): e17140). In this method a limbal conjunctival peritomy is performed to expose bare sclera, followed by sclerotomy to expose bare choroid. A microcatheter (such as the iTrack 250A from iScience Interventional, optionally connected to an illumination system such as the iLumin laser-diode based micro-illumination system (iScience Interventional)) is introduced into the suprachoroidal space and advanced posteriorly towards the optic disc. Following manipulation of the microcatheter tip into the desired position, injection of the product, polynucleotide or vector forms a bleb within the retina and choroid.

Thus, in some embodiments, the product, polynucleotide or vector is delivered suprachoroidally by a method comprising (i) introduction of a microcatheter into the suprachoroidal space; (ii) advancing the microcatheter within said space until the tip is in the proximity of the afflicted region of the retina; and (iii) injecting the product, polynucleotide or vector from the microcatheter tip to create a bleb.

In some embodiments, the above administration procedures are directly carried out by a robot.

Pharmaceutical Compositions and Injected Solutions

The medicaments, for example products, polynucleotides or vectors, of the invention may be formulated into pharmaceutical compositions. These compositions may comprise, in addition to the medicament, a pharmaceutically acceptable carrier, diluent, excipient, buffer, stabiliser or other materials well known in the art. Such materials should be non-toxic and should not interfere with the efficacy of the active ingredient. The precise nature of the carrier or other material may be determined by the skilled person according to the route of administration, e.g. subretinal, direct retinal, suprachoroidal or intravitreal injection.

The pharmaceutical composition is typically in liquid form. Liquid pharmaceutical compositions generally include a liquid carrier such as water, petroleum, animal or vegetable oils, mineral oil or synthetic oil. Physiological saline solution, magnesium chloride, dextrose or other saccharide solution, or glycols such as ethylene glycol, propylene glycol or polyethylene glycol may be included. In some cases, a surfactant, such as pluronic acid (PF68) 0.001% may be used.

For injection at the site of affliction, the active ingredient may be in the form of an aqueous solution which is pyrogen-free, and has suitable pH, isotonicity and stability. The skilled person is well able to prepare suitable solutions using, for example, isotonic vehicles such as Sodium Chloride Injection, Ringer's Injection or Lactated Ringer's Injection. Preservatives, stabilisers, buffers, antioxidants and/or other additives may be included as required.

For delayed release, the medicament may be included in a pharmaceutical composition which is formulated for slow release, such as in microcapsules formed from biocompatible polymers or in liposomal carrier systems according to methods known in the art.

Method of Treatment

It is to be appreciated that all references herein to treatment include curative, palliative and prophylactic treatment; although in the context of the invention references to preventing are more commonly associated with prophylactic treatment. Treatment may also include arresting progression in the severity of a disease.

The treatment of mammals, particularly humans, is preferred. However, both human and veterinary treatments are within the scope of the invention.

The term “combination”, or terms “in combination”, “used in combination with” or “combined preparation” as used herein may refer to the combined administration of two or more agents simultaneously, sequentially or separately.

The term “simultaneous” as used herein means that the agents are administered concurrently, i.e. at the same time.

The term “sequential” as used herein means that the agents are administered one after the other.

The term “separate” as used herein means that the agents are administered independently of each other but within a time interval that allows the agents to show a combined, preferably synergistic, effect. Thus, administration “separately” may permit one agent to be administered, for example, within 1 minute, 5 minutes or 10 minutes after the other.

Variants, Derivatives, Analogues, Homologues and Fragments

In addition to the specific proteins and nucleotides mentioned herein, the invention also encompasses the use of variants, derivatives, analogues, homologues and fragments thereof.

In the context of the invention, a variant of any given sequence is a sequence in which the specific sequence of residues (whether amino acid or nucleic acid residues) has been modified in such a manner that the polypeptide or polynucleotide in question substantially retains its function. A variant sequence can be obtained by addition, deletion, substitution, modification, replacement and/or variation of at least one residue present in the naturally-occurring protein.

The term “derivative” as used herein, in relation to proteins or polypeptides of the invention includes any substitution of, variation of, modification of, replacement of, deletion of and/or addition of one (or more) amino acid residues from or to the sequence providing that the resultant protein or polypeptide substantially retains at least one of its endogenous functions.

The term “analogue” as used herein, in relation to polypeptides or polynucleotides includes any mimetic, that is, a chemical compound that possesses at least one of the endogenous functions of the polypeptides or polynucleotides which it mimics.

Typically, amino acid substitutions may be made, for example from 1, 2 or 3 to 10 or 20 substitutions provided that the modified sequence substantially retains the required activity or ability. Amino acid substitutions may include the use of non-naturally occurring analogues.

Proteins used in the invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and preferably in the same line in the third column may be substituted for each other:

| | | |
|---|---|---|
| ALIPHATIC | Non-polar | GAP |
| | | ILV |
| | Polar-uncharged | CSTM |
| | | NQ |
| | Polar-charged | DE |
| | | KRH |
| AROMATIC | | FWY |

The term “homologue” as used herein means an entity having a certain homology with the wild type amino acid sequence and the wild type nucleotide sequence. The term “homology” can be equated with “identity”.

A homologous sequence may include an amino acid sequence which may be at least 50%, 55%, 60%, 65%, 70%, 75%, 80%, 85% or 90% identical, preferably at least 95% or 97% or 99% identical to the subject sequence. Typically, the homologues will comprise the same active sites etc. as the subject amino acid sequence. Although homology can also be considered in terms of similarity (i.e. amino acid residues having similar chemical properties/functions), in the context of the invention it is preferred to express homology in terms of sequence identity.

A homologous sequence may include a nucleotide sequence which may be at least 50%, 55%, 60%, 65%, 70%, 75%, 80%, 85% or 90% identical, preferably at least 95% or 97% or 99% identical to the subject sequence. Although homology can also be considered in terms of similarity, in the context of the invention it is preferred to express homology in terms of sequence identity.

Preferably, reference to a sequence which has a percent identity to any one of the SEQ ID NOs detailed herein refers to a sequence which has the stated percent identity over the entire length of the SEQ ID NO referred to.

Homology comparisons can be conducted by eye or, more usually, with the aid of readily available sequence comparison programs. These commercially available computer programs can calculate percentage homology or identity between two or more sequences.

Percentage homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the nucleotide sequence may cause the following codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percentage homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Res. 12: 387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, called BLAST 2 Sequences is also available for comparing protein and nucleotide sequences (see FEMS Microbiol. Lett. (1999) 174: 247-50; FEMS Microbiol. Lett. (1999) 177: 187-8).

Although the final percent homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62.

Once the software has produced an optimal alignment, it is possible to calculate percent homology, preferably percent sequence identity. The software typically does this as part of the sequence comparison and generates a numerical result.

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleotide.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein.

These methods are only illustrative of the numerous standard techniques known in the art for manipulation of DNA sequences and other known techniques may also be used.

Aspects of the Invention

Aspects of the invention are defined in the following numbered paragraphs (paras):
1. A product comprising (i) an anti-VEGF entity; and (ii) a negative complement regulator, or nucleotide sequences encoding therefor, as a combined preparation for simultaneous, separate or sequential use in therapy.
2. A product for use according to para 1 wherein the disease to be treated or prevented is an ocular disorder.
3. A product for use according to para 2 wherein the ocular disorder is a complement-mediated disorder of the eye.
4. The product for use according to para 3, wherein the complement-mediated disorder is age-related macular degeneration (AMD), diabetic retinopathy, glaucoma, Stargardt's disease, central serous chorioretinopathy, retinitis pigmentosa, polypoidal choroidal vasculopathy, diabetic macular edema, branch retinal vein occlusion or uveitis, preferably AMD.
5. The product for use according to para 4, wherein the AMD is wet AMD and/or dry AMD.
6. The product for use according to para 4, wherein the AMD is wet AMD and the use further prevents and/or treats onset of dry AMD in said subject.
7. The product for use according to any of paras 1 to 6 wherein the product comprises a polynucleotide comprising nucleotide sequences encoding (i) an anti-VEGF entity and (ii) a negative complement regulator.
8. An isolated polynucleotide comprising nucleotide sequences encoding (i) an anti-VEGF entity and (ii) a negative complement regulator.
9. A product for use according to para 7 or an isolated polynucleotide according to para 8 wherein the anti-VEGF entity is selected from an Ig fusion protein, an antibody, a polypeptide, a peptide, a non-antibody scaffold, an antisense oligonucleotide, siRNA, shRNA, CRISPR-guide strand and an aptamer.
10. A product for use or an isolated polynucleotide according to para 9 wherein the anti-VEGF entity is selected from aflibercept, ranibizumab, bevacizumab and pegaptanib.
11. An isolated polynucleotide according to para 10 wherein the anti-VEGF entity aflibercept, for example wherein the aflibercept is encoded by a polynucleotide sequence comprising any one of SEQ ID NO: 3 to 11.
12. A product for use or an isolated polynucleotide according to any preceding para wherein the negative complement regulator is selected from Complement Factor I (CFI), Complement Factor H Like Protein 1 (FHL1), Complement Factor H (CFH), Complement receptor type 1 (CR1), Membrane Cofactor Protein (MCP), Complement decay-accelerating factor (DAF), MAC-inhibitory protein (MAC-IP), C1-inhibitor, anaphylatoxins inhibitor, C4b binding protein (C4BP), clusterin, vitronectin or a variant or fragment thereof.
13. A product for use or an isolated polynucleotide according to para 12 wherein the negative complement regulator is selected from CFI and FHL1 or a variant or fragment thereof.
14. The product for use or isolated polynucleotide according to any of paras 7 to 13, wherein the polynucleotide further comprises nucleotide sequences encoding:
   (a) a CMV or CAG promoter, optionally wherein the CMV or CAG promoter is upstream of the nucleotide sequences encoding the (i) and (ii);
   (b) a WPRE regulatory element, optionally wherein the WPRE regulatory element is downstream of the nucleotide sequences encoding the (i) and (ii); and/or
   (c) a poly-A signal, optionally a Bovine Growth Hormone poly-A signal, wherein the polyA signal is optionally downstream of the nucleotide sequences encoding the (i) and (ii).
15. The product for use or isolated polynucleotide according to any of paras 7 to 14, wherein the polynucleotide further comprises one or more adeno-associated virus (AAV) inverted terminal repeats (ITRs).
16. The product for use or isolated polynucleotide according to any of paras 7 to 15, wherein the polynucleotide comprises an AAV ITR at its 5′ end and an AAV ITR at its 3′ end.
17. The product for use or isolated polynucleotide according to any of paras 7 to 16, wherein the polynucleotide comprises:
   (a) a 5′ AAV ITR;
   (b) a CMV or CAG promoter;
   (c) a nucleotide sequence encoding an anti-VEGF entity;
   (d) a linker, optionally wherein the linker is comprises or is defined by a Furin cleavage site, GSG, 11aa1 D sequence or an F2A sequence;
   (e) a nucleotide sequence encoding a negative complement regulator;
   (f) optionally, a WPRE regulatory element, further optionally wherein the WPRE regulatory element is a WPRE3 regulatory element;
   (g) a Bovine Growth Hormone poly-A signal; and
   (h) a 3′ AAV ITR.
18. The product for use or isolated polynucleotide according to any of paras 7 to 17, wherein the AAV ITRs are AAV2 ITRs.
19. The product for use or isolated polynucleotide according to any of paras 7 to 18, wherein the nucleotide sequences encoding the anti-VEGF entity and the negative complement regulator are codon optimised.
20. The product for use or isolated polynucleotide according to any of paras 7 to 19, wherein the nucleotide sequence encoding aflibercept has at least 75% sequence identity to SEQ ID NO: 11.
21. The product for use or isolated polynucleotide according to any of paras 7 to 20, wherein the nucleotide sequence encoding CFI has at least 75% sequence identity to SEQ ID NO: 35 or 36 or the nucleotide sequence encoding FHL-1 has at least 75% sequence identity to SEQ ID NO: 41.
22. The product for use or isolated polynucleotide according to any of paras 7 to 20, wherein the polynucleotide comprises the nucleotide sequence of SEQ ID NO: 45 to 54, or a nucleotide sequence that has at least 75% sequence identity thereto.
23. The product for use or isolated polynucleotide according to any of paras 7 to 22, wherein the polynucleotide is less than or equal to 4.7 kb.
24. A vector comprising the polynucleotide of any one of paras 8-23.
25. The vector of para 24, wherein the vector is an adeno-associated viral (AAV) vector.
26. The vector of para 24 or 25, wherein the vector is in the form of a viral vector particle.
27. The vector of para 26, wherein the AAV vector particle comprises an AAV2 or AAV8 genome, and AAV2 or AAV8 capsid proteins.
28. A cell comprising the polynucleotide of any one of paras 7 to 22.
29. A cell transduced with the vector of any one of paras 24-27.
30. A pharmaceutical composition comprising the isolated polynucleotide, vector or cell of any one of paras 8-29 in combination with a pharmaceutically acceptable carrier, diluent or excipient.
31. The isolated polynucleotide, vector or cell of any one of paras 8-29 for use in therapy.
32. The isolated polynucleotide, vector or cell of any one of paras 8-29 for use in treating or preventing a complement-mediated disorder of the eye.
33. The isolated polynucleotide, vector or cell for use according to para 32, wherein the complement-mediated disorder is age-related macular degeneration (AMD), diabetic retinopathy, glaucoma, Stargardt's disease, central serous chorioretinopathy, retinitis pigmentosa, polypoidal choroidal vasculopathy, diabetic macular edema, branch retinal vein occlusion or uveitis, preferably AMD.
34. The isolated polynucleotide, vector or cell for use according to para 32, wherein the AMD is wet AMD and/or dry AMD.
35. The isolated polynucleotide, vector or cell for use according to para 33, wherein the AMD is wet AMD and the use further prevents and/or treats onset of dry AMD in said subject,
36. The polynucleotide, vector or cell for use according to any one of paras 32-35, wherein the formation of geographic atrophy is prevented or reduced, and/or the amount of geographic atrophy is reduced.
37. The polynucleotide, vector or cell for use according to any one of paras 32-36, wherein the progression of geographic atrophy is slowed.
38. The polynucleotide, vector or cell for use according to any one of paras 32-37, wherein there is at least a 10% reduction in the increase in geographic atrophy area over the 12 months following administration to a treated eye of a subject, relative to an untreated eye over the same period.
39. The polynucleotide, vector or cell for use according to any one of paras 32-38, wherein administration of the polynucleotide, vector or cell increases the level of C3b-inactivating and iC3b-degradation activity in a subject, or in an eye, such as in the retinal pigment epithelium (RPE), of a subject, optionally to a level that exceeds a normal level in a subject, or eye or RPE thereof.
40. The polynucleotide, vector or cell for use according to any one of paras 32-39, wherein the polynucleotide, vector or cell is administered intraocularly.
41. The polynucleotide, vector or cell for use according to any one of paras 32-40, wherein the polynucleotide, vector or cell is administered to the eye of a subject by subretinal, direct retinal, suprachoroidal or intravitreal injection.
42. The polynucleotide, vector or cell for use according to any one of paras 32-41, wherein the polynucleotide, vector or cell is administered to the eye of a subject by subretinal injection.
43. A method of treating or preventing a complement-mediated disorder of the eye comprising administering the isolated polynucleotide, vector or cell of any one of paras 8-29 to a subject in need thereof.
44. A method of providing (i) an anti-VEGF entity; and (ii) a negative complement regulator to a subject, comprising delivering the isolated polynucleotide, vector or cell of any one of paras 8-29 to the eye of the subject.
45. An anti-VEGF entity for use in treating or preventing a disease, wherein the anti-VEGF entity is used in combination with a negative complement regulator.
46. A negative complement regulator for use in treating or preventing a disease, wherein the negative complement regulator is used in combination with an anti-VEGF entity.
47. An anti-VEGF entity for use according to para 45 or a negative complement regulator for use according to para 46, wherein the disease is a disease as recited in any of paras 2 to 6.
48. An anti-VEGF entity for use according to para 45 or 47 wherein the anti-VEGF entity is an entity as defined in any of paras 9 to 11.
49. A negative complement regulator for use according to para 46 or 47 wherein the negative complement regulator is an entity as defined in para 12 or 13.

The skilled person will understand that they can combine all features of the invention disclosed herein without departing from the scope of the invention as disclosed.

Preferred features and embodiments of the invention will now be described by way of non-limiting examples.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E. F. and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J. M. and McGee, J. O'D. (1990) In Situ Hybridization: Principles and Practice, Oxford University Press; Gait, M. J. (1984) Oligonucleotide Synthesis: A Practical Approach, IRL Press; and Lilley, D. M. and Dahlberg, J. E. (1992) Methods in Enzymology: DNA Structures Part A: Synthesis and Physical Analysis of DNA, Academic Press. Each of these general texts is herein incorporated by reference.

### EXAMPLES

### Example 1—Codon Optimisation of Aflibercept Sequence

The aflibercept protein sequence was first obtained from www.drugbank.ca (see SEQ ID NO: 1).

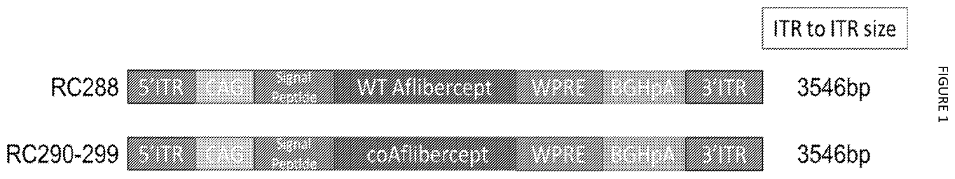

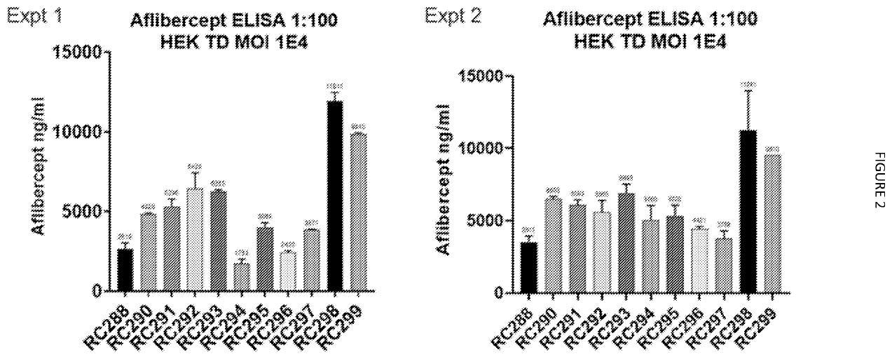

The protein sequence from ‘Drugbank’ was back translated into DNA sequence, which was then aligned with human VEGF Receptor and human IgG protein sequence and denoted as the ‘wildtype’ Aflibercept sequence. The wildtype aflibercept DNA sequence was derived and subconed into GT005 backbone denoted as RC288. A native FHL-1 signal peptide was added upstream of the wildtype aflibercept sequence to allow secretion from cells. Five online tools were used to generate “basic” sequences (GeneArt, Genscript, Genewiz, IeT & JCat). Each of the basic sequences were then manually optimised to remove cryptic splice sites, miRNA binding sites and tandem duplicate codons. These optimised sequences were designated as “manual”. Each codon optimised sequence was synthesised and cloned into GT005 backbone by Genewiz (Table 1 and FIG. 1).

TABLE 1
Table enlisting variations of codon optimised Aflibercept
constructs and reference links for online codon optimisation tools

| Variant | | Codon | |
|---|---|---|---|
| Number | Plasmid | Optimisation | |
| # | Code | Tools | Reference Links |
| Wild | RC288 | Original | Aflibercept sequence derived from |
| type | | “wild type” | drugbank.ca |
| 1 | RC290 | GeneArt | https://www.thermofisher.com/uk/en/home/life- |
| | | Basic | science/cloning/gene-synthesis/geneart-gene- |
| | | | synthesis/geneoptimizer.html |
| 2 | RC291 | GeneArt | |
| | | Manual | |
| 3 | RC292 | Genscript | https://www.genscript.com/ |
| | | Basic | |
| 4 | RC293 | Genscript | |
| | | Manual | |
| 5 | RC294 | GeneWiz | https://www.genewiz.com/en-GB/ |
| | | Basic | |
| 6 | RC295 | Genewiz | |
| | | Manual | |
| 7 | RC296 | IDT Basic | https://eu.idtdna.com/pages/tools/codon- |
| | | | optimization-tool?returnurl=%2FCodonOpt |
| 8 | RC297 | IDT Manual | |
| 9 | RC298 | JCat Basic | http://www.jcat.de/ |
| 10 | RC299 | JCat Manual | |

In general, the monocistronic Aflibercept vector is composed of:
Inverted terminal repeats (ITR)
CMV early enhancer/chicken R-actin (CAG) promoter
Native or codon optimised FHL1 secretion signal peptide
Aflibercept is composed of sequences from the second binding domain of vascular endothelial growth factor receptor VEGFR-1 and the third binding domain of VEGFR-2 fused to the sequence of a FC fragment of a human IgG (WT=wild type; coAflibercept=codon optimised Aflibercept sequence)
Woodchuck hepatitis virus post-transcriptional regulatory element (WPRE)
Bovine growth hormone poly-adenylation signal (bGHpA)

### Example 2—Comparison of Vectorised Aflibercept Expression In Vitro

All codon optimised Aflibercept vectors were packaged into AAV2 using triple transfection method. All vectors were then titred by qPCR and transduced into HEK293 at MOI 1 E4. The level of Aflibercept expression was measured in culture supernatants 72 hr post-transduction by ELISA. The experiment was repeated twice. As shown in FIG. 2, all codon optimised Aflibercept vectors showed varying levels of expression.

In particular, RC298 (Jcat Basic) and RC299 (Jcat Manual) showed the highest level of Aflibercept expression as compared to control RC288 vector containing the non-codon optimised wild type sequence.

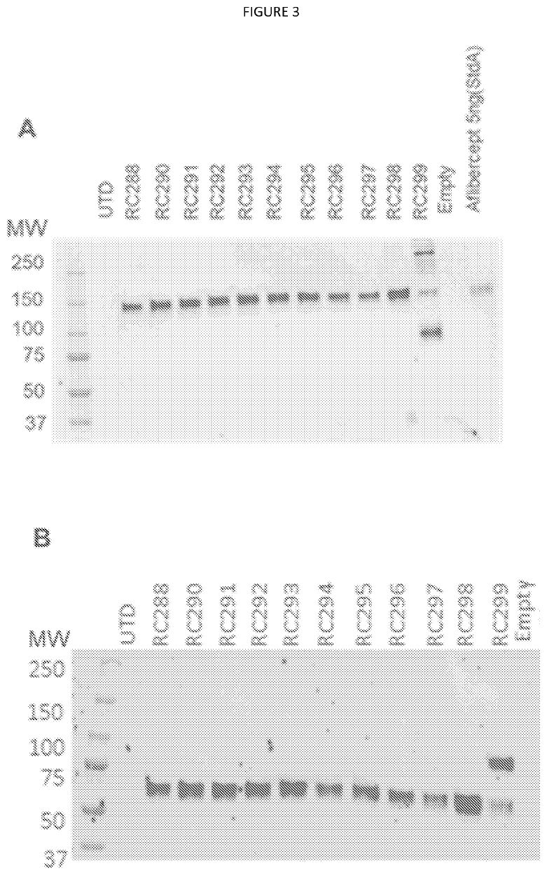

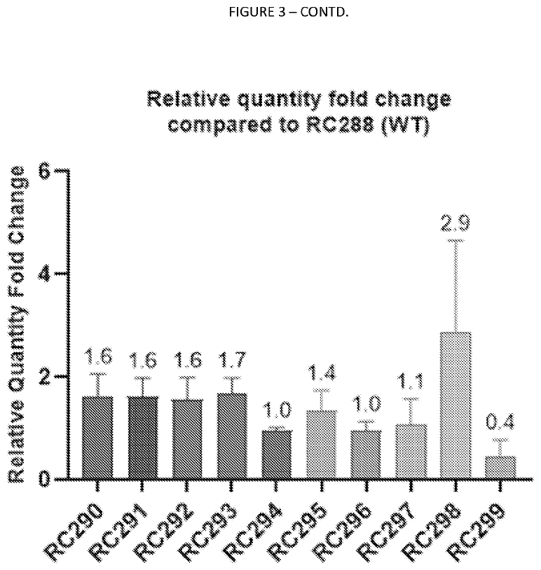

Western blot analysis was also carried out to confirm the integrity of the expressed Aflibercept protein from the AAV2 vectors. All vectors demonstrated Aflibercept protein expression at the expected molecular weight of 115 kDa (under non-reducing conditions), which was comparable to recombinant Aflibercept protein (FIGS. 3A and B). Densitometric analysis showed that RC298 expressed the highest level of Aflibercept (FIG. 3C), consistent with the ELISA data as per FIG. 2. In particular, RC299 showed an aberrant protein band pattern, and was therefore excluded from selection.

In conclusion, RC298 was selected as the most optimal codon optimised Aflibercept sequence to take forward into the bicistronic vector.

### Example 3—Construction of Bicistronic Vectors Co-Expressing Negative Complement Regulator and Aflibercept

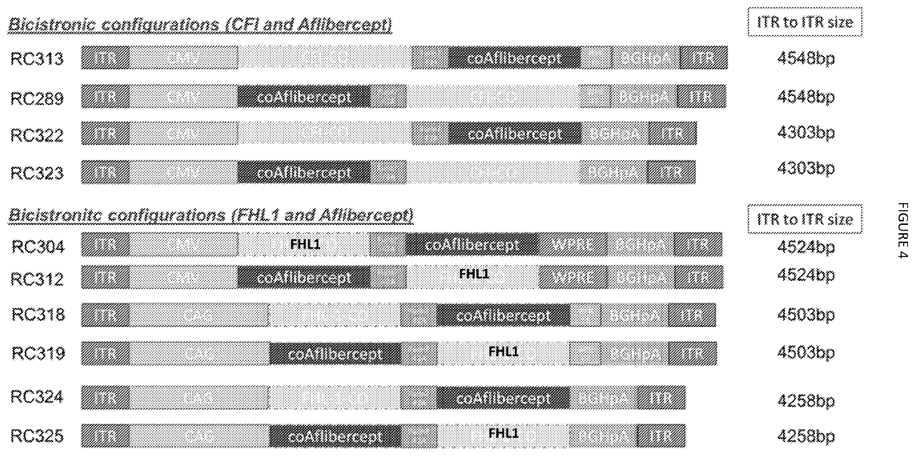

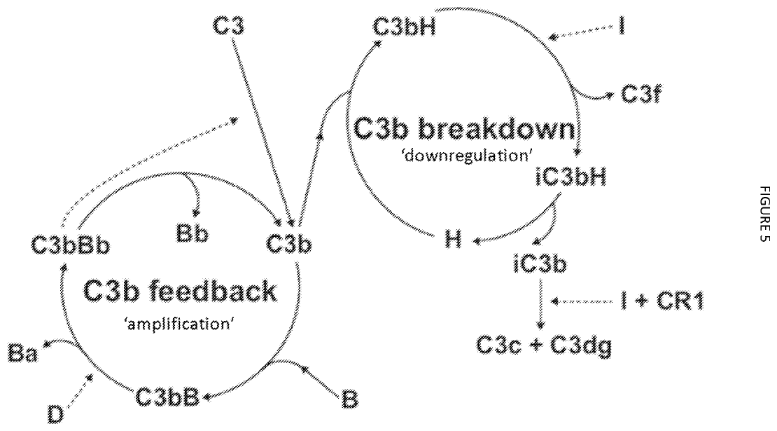

Bicistronic vectors were generated composed of a (CAG or CMV) promoter driving the expression of either codon optimised CFI or codon optimised FHL1 linked to codon optimised Aflibercept (derived from RC298) linked by a furin F2A linker. The position of the negative complement regulator and Alifbercept was interchanged between different configurations as shown in FIG. 4 and Table 2.

In general, the bicistronic Aflibercept vectors are composed of:
Inverted terminal repeats (ITR)
Cytomegalovirus (CMV) promoter or CMV early enhancer/chicken p-actin (CAG) promoter
Codon optimised complement factor I (CFI) or codon optimised FHL1
Furin F2A cleavage peptide
Aflibercept—composed of sequences from the second binding domain of vascular endothelial growth factor receptor VEGFR-1 and the third binding domain of VEGFR-2 fused to the sequence of a FC fragment of a human IgG (WT=wild type; coAflibercept=codon optimised Aflibercept sequence)
Woodchuck hepatitis virus post-transcriptional regulatory element (WPRE) or shortened version of WPRE containing only minimal gamma and alpha elements (WPRE3)
Bovine growth hormone poly-adenylation signal (bGHpA)

TABLE 2
Table enlisting bicistronic vectors and corresponding configurations

| | Negative | | | |
|---|---|---|---|---|
| Plasmid | Complement | | Description of | SEQ |
| Code | Regulator | Anti-VEGF | Packaged genome | ID NO |
| RC313 | CFI (codon | Codon optimised | ITR-CMV-coCFI- | 45 |
| | optimised) | Aflibercept | F2A-coAflibercept- | |
| | | sequence from | WPRE3- | |
| | | RC298 | bGHpA-ITR | |
| RC289 | CFI (codon | Codon optimised | ITR-CMV- | 46 |
| | optimised) | Aflibercept | coAflibercept-F2A- | |
| | | sequence from | coCFI-WPRE3- | |
| | | RC298 | bGHpA-ITR | |
| RC322 | CFI (codon | Codon optimised | ITR-CMV-coCFI- | 47 |
| | optimised) | Aflibercept | F2A-coAflibercept- | |
| | | sequence from | bGHpA-ITR | |
| | | RC298 | | |
| RC323 | CFI (codon | Codon optimised | ITR-CMV- | 48 |
| | optimised) | Aflibercept | coAflibercept-F2A- | |
| | | sequence from | coCFI-bGHpA-ITR | |
| | | RC298 | | |
| RC304 | FHL1 (codon | Codon optimised | ITR-CMV-coFHL1- | 49 |
| | optimised) | Aflibercept | F2A-coAflibercept- | |
| | | sequence from | WPRE- | |
| | | RC298 | bGHpA-ITR | |
| RC312 | FHL1 (codon | Codon optimised | ITR-CMV- | 50 |
| | optimised) | Aflibercept | coAflibercept-F2A- | |
| | | sequence from | coFHL1-WPRE- | |
| | | RC298 | bGHpA-ITR | |
| RC318 | FHL1 (codon | Codon optimised | ITR-CAG-coFHL1- | 51 |
| | optimised) | Aflibercept | F2A-coAflibercept- | |
| | | sequence from | WPRE3- | |
| | | RC298 | bGHpA-ITR | |
| RC319 | FHL1 (codon | Codon optimised | ITR-CAG- | 52 |
| | optimised) | Aflibercept | coAflibercept-F2A- | |
| | | sequence from | coFHL1-WPRE3- | |
| | | RC298 | bGHpA-ITR | |
| RC324 | FHL1 (codon | Codon optimised | ITR-CAG-coFHL1- | 53 |
| | optimised) | Aflibercept | F2A-coAflibercept- | |
| | | sequence from | bGHpA-ITR | |
| | | RC298 | | |
| RC325 | FHL1 (codon | Codon optimised | ITR-CAG- | 54 |
| | optimised) | Aflibercept | coAflibercept- | |
| | | sequence from | F2A-coFHL1- | |
| | | RC298 | bGHpA-ITR | |

The codon optimised CFI sequence is SEQ ID NO: 35.

The codon optimised FHL1 sequence is SEQ ID NO: 41.

The codon optimised aflibercept sequence is SEQ ID NO: 11.

The 5′ ITR is SEQ ID NO: 55.

The CMV promoter is SEQ ID NO: 59

The CAG promoter is SEQ ID NO: 60.

The WPRE sequence is SEQ ID NO: 63.

The WPRE3 sequence is SEQ ID NO: 64.

The bGHpA sequence is SEQ ID NO: 61.

The 3′ ITR is SEQ ID NO: 57.

### Example 4—Functional Characterisation of Bicistronic Vectors Co-Expressing Negative Complement Regulator and Aflibercept

Comparison of bicistronic vector expression was performed by in vitro transduction to compare expression of Aflibercept, CFI and FHL1 from bicistronic vectors listed in FIG. 4 against monocistronic counterparts.

Functional analysis of Aflibercept from the bicistronic vectors was performed to determine anti-VEGF binding and anti-proliferation capabilities.

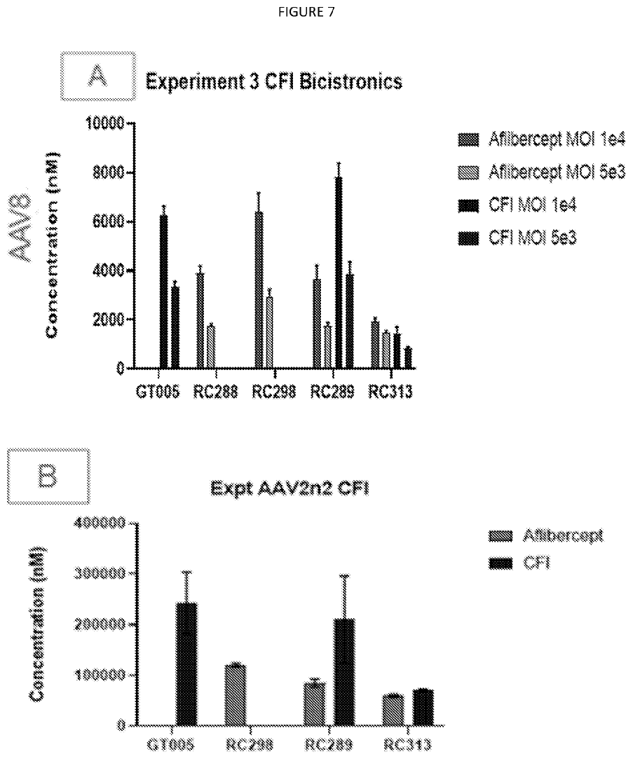

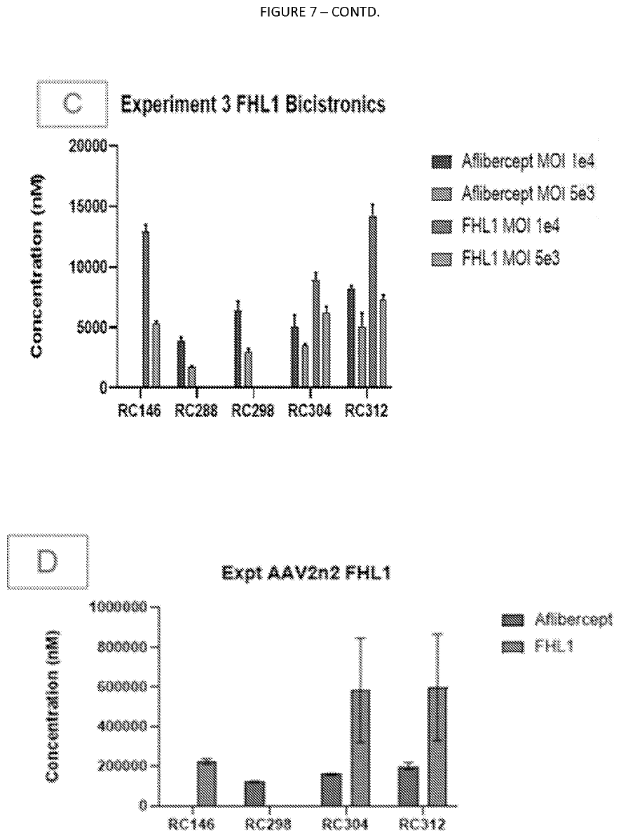

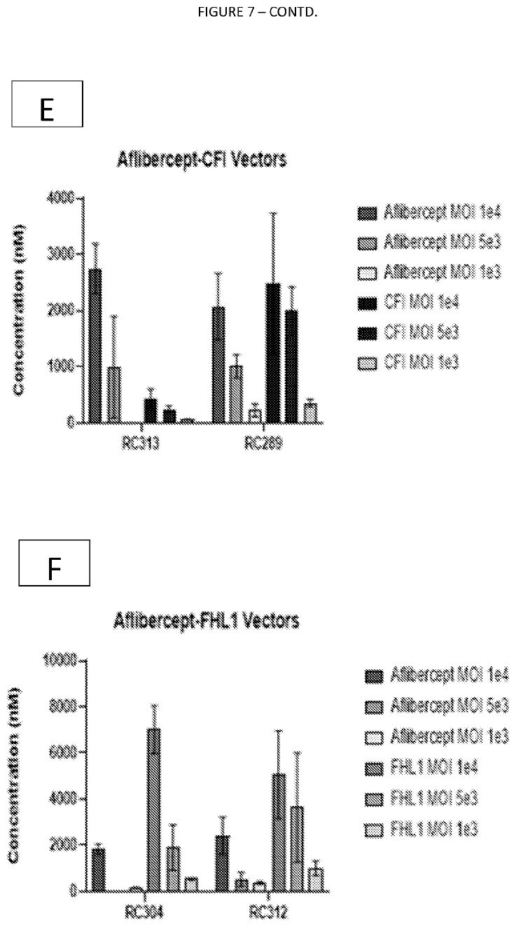

All bicistronic vectors were either packaged into AAV8 or AAV2 using triple transfection method. In vitro transduction was carried out in HEK293 cells at different MOIs to compare the expression of respective transgenes from bicistronic vectors as listed in Table 2. ELISA was used to quantify the expression levels of Aflibercept, CFI and FHL1 from the respective bicistronic vectors. In parallel, expression was compared to monocistronic counterparts. AAV8 and AAV2 bicistronic vectors (RC289 and RC313) showed measurable levels of CFI and Aflibercept expression following transduction, and expression levels from RC289 packaged as AAV8 and AAV2 vectors were comparable to monocistronic counterparts (FIGS. 7A and B). In contrast, RC313 showed lower levels of expression compared to RC289 and corresponding monocistronic vectors. Similarly, AAV8 and AAV2 bicistronic vectors (RC304 and RC312) showed quantifiable levels of FHL-1 and Aflibercept expression and the levels of expression were comparable to that of corresponding monocistronic vectors (RC146—monocistronic FHL1, RC298—monocistronic Aflibercept) (FIGS. 7C and D).

In a further experiment, bicistronic vectors RC313, RC289, RC304 and RC312 were packaged into AAV8 using triple transfection method. Purified vectors were transduced in HEK293 at 3 MOIs, in triplicate. Supernatants were taken and quantified by Aflibercept, CFI & FHL-1 ELISAs. For the CFI-Aflibercept bicistronic vectors (RC313 and RC289), Aflibercept expression was similar between the two vectors, and RC289 showed higher CFI expression than RC313 (FIG. 7E). For the FHL-1-Aflibercept bicistronic vectors, Aflibercept expression was again similar between the vectors, and RC304 showed higher FHL-1 expression at MOI 1 E4 as compared to RC312 (FIG. 7F). Using the same cell culture supernatant samples from expression studies, western blot analysis was carried out to assess protein expression from all bicistronic vectors. Under non-reducing conditions all cell culture supernatants showed CFI, FHL-1 and Aflibercept protein expression and all bicistronic vectors expressed the expected protein size similar to that of the recombinant protein controls (see FIG. 8).

These results indicate that all bicistronic vectors tested enabled dual transgene expression, and some candidates showed expression levels similar to that of monocistronic vectors. All bicistronic vectors expressed proteins of the correct size.

VEGF Binding Assay

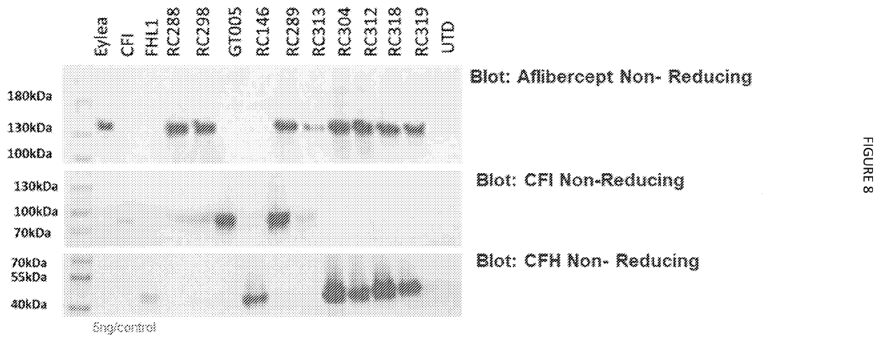

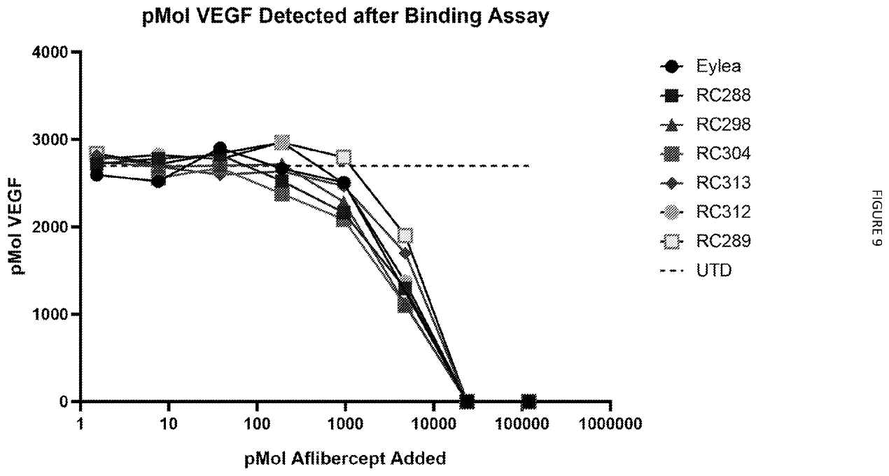

To determine binding affinity of Aflibercept expressed from the bicistronic vectors for VEGFA, an equilibrium binding assay was performed. HEK293 cells were transduced with monocistronic vectors expressing Aflibercept (RC298), and bicistronic vectors co-expressing CFI or FHL1 and Aflibercept. 72 hours post-transduction, secreted Aflibercept in the culture supernatant was extracted and quantified by ELISA. Binding affinities of secreted Aflibercept derived from vectors and recombinant Aflibercept (Eylea®) was measured by using a specific and sensitive ELISA for detecting free (unbound) human VEGFA in mixtures of the Aflibercept (across a range of concentration) with human VEGFA incubated over a fixed period of time. As shown in FIG. 9, the binding equilibriums (kD) of Aflibercepts [IC50] for VEGFA derived from both monocistronic and bicistronic vectors were found to be similar to that of Eylea®.

These data demonstrate that the designed bicistronic vectors were able to express Aflibercept that functions at a similar capacity to that of Eylea®.

VEGF-Induced Proliferation Assay

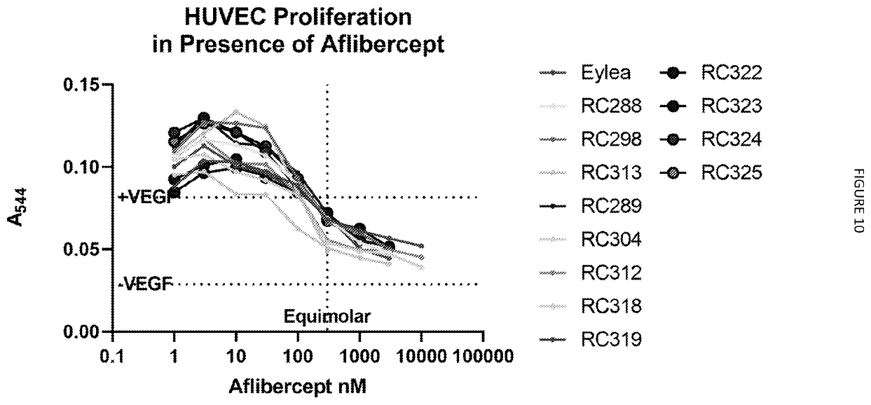

To determine whether AAV vector derived Aflibercept effectively binds VEGFA and effectively blocks the ability of VEGF to induce cell proliferation, human umbilical vascular endothelial (HUVEC) cells were challenged with a set concentration of human VEGFA and varying concentrations of Aflibercept (derived from cultured supernatant following AAV transduction of HEK293 cells as above) or Eylea® (positive control). 72 hr post-treatment, inhibition of VEGF-induced proliferation of HUVEC cells was measured by the addition of [3-(4,5 dimethylthiazol-2-yl)-5-(3-carboxymethoxypheyl)-2-(4-sulfophenyl)-2H-tetrazolium, and spectrophotometric analysis at 450/570 nm. As shown in FIG. 10, Aflibercept derived from monocistronic and bicistronic vectors blocked VEGF-induced proliferation in a 5-day growth assays in these cells, and the anti-proliferative properties observed were similar to that of Eylea®.

These data demonstrate that the designed bicistronic vectors are able to express Aflibercept that functions at a similar capacity to that of Eylea®.

C3b Cleavage Assays

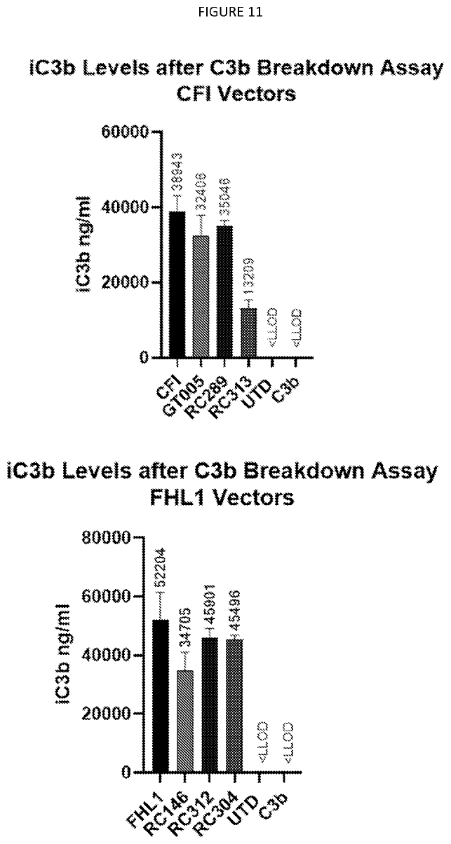

To analyse the functional activity of CFI or FHL1 secreted from transduced cells, conditioned supernatant from HEK293 cells transduced with: AAV2 expressing CFI (GT005); AAV2 expressing FHL1 (RC146); AAV2 co-expressing CFI and Aflibercept (RC289 or RC313), or AAV2 co-expressing FHL1 and Aflibercept (RC312 or RC304) was tested in a C3b cleavage assay (FIG. 11). The principle of this assay is based on the ability of CFI, in the presence of FHL1, to cleave C3b to iC3b. iC3b ELISA quantifies the amount of C3b breakdown product, iC3b.

C3b cleavage assays were conducted by incubating 1 ug C3b with recombinant FHL1 (GTP Tech) and supernatant from cells transduced with CFI expressing vectors (GT005, RC289 and RC313) at a 4:1 molar ratio of FHL1: CFI. The reaction volume was made up to 20 ul with Opti-MEM™ reduced serum media and reactions were incubated at 37 degrees for 20 minutes. Supernatant from cells transduced with FHL1 vectors (RC146, RC312 or RC304) was incubated with recombinant CFI (Complement Technology, Inc, A138), 1 ug C3b (Complement Technology, Inc, A113) and Opti-MEM™ serum free media at a 4:1 molar ratio of FHL1: CFI at 37° C. for 20 minutes in a total volume of 20 ul.

Resultant samples were diluted 1:100 and 1:400 and loaded onto an iC3b ELISA using coating antibody: mouse anti-human activated C3 (Hycult, HM2168), detection antibody: rat anti-C3dg (Hycult, HM2199), and secondary antibody: mouse anti-rat (Southern Biotech, 3061-05). iC3b concentrations (ng/mL) were calculated according to a standard curve generated using iC3b purified human protein (Complement Technology, Inc).

C3b incubated with recombinant CFI and FHL1 served as a positive control (FIG. 11, left hand bars). The C3b only negative control demonstrated that C3b breakdown requires the presence of CFI and FHL1 as C3b is stable in the absence of additional components. C3b incubated with conditioned supernatant from untransduced cells (UTD) also served as a negative control. iC3b levels from bicistronic vectors were compared to monocistronic counterparts. Results of iC3b ELISA demonstrated that CFI and FHL1 expressed from all bicistronic vectors is functional and can facilitate breakdown of C3b to iC3b (see FIG. 11).

In Vivo Studies

In vivo bicistronic expression and efficacy studies are performed to determine expression of negative complement regulator and Aflibercept from bicistronic vectors in rodents. Studies are also performed to demonstrate efficacy of bicistronic vectors in a laser CNV mouse model.

### Example 5—Administration of Monocistronic Vectors Expressing CFI or FHL1 in a Mouse Model of CNV

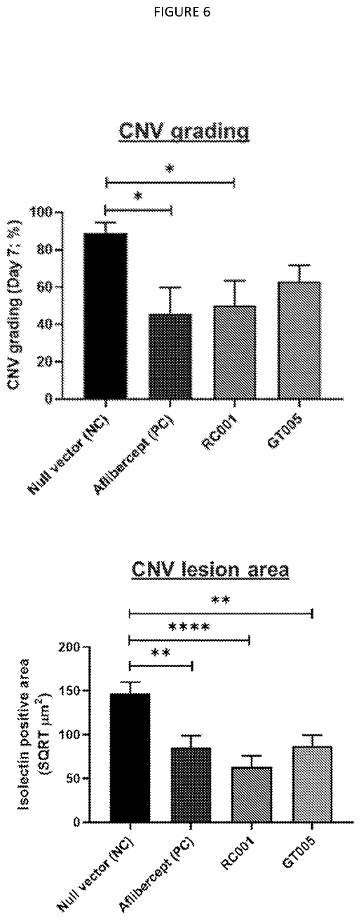

AAV vectors are first delivered subretinally into the mouse eye and left for 4 weeks. At Day 0, all pre-injected eyes are subjected to laser induced CNV lesions by laser burning of the Bruch's membrane. Aflibercept (Eylea®) is also delivered via intravitreal injection into CNV lesion eyes for the positive control group. Optical coherence tomography and fluorescein angiography are used to image the retinal structure and CNV lesion areas respectively at Day 0 and Day 4. At Day 7 post-laser burn, all mice are culled and CNV areas (determined by isolectin staining), CNV leakage area and CNV score (graded) are measured as primary and secondary endpoints (see FIG. 6).

### Example 6—In Vivo Expression Studies

Monocistronic Expression Data

AAV8 (monocistronic) vectors expressing anti-VEGF (Aflibercept) were administered subretinally into the right eye of nine-week-old C57BL/6JRj male mice as per groups below. Successful subretinal administrations were verified using spectral-domain optical coherence tomography (SD-OCT)
Group 1: AAV8 RC298, dose 5 e7 vg/eye (n=9);
Group 2: AAV8 RC298, dose 2 e8 vg/eye (n=10);
Group 3: AAV8 RC298, dose 5 e8 vg/eye (n=10);
Group 4: AAV8 RC298, dose 2 e9 vg/eye (n=10);
Group 5: AAV8 RC298, dose 5 e9 vg/eye (n=10);
Group 6: AAV8 RC298, dose 1 e10 vg/eye (n=9).

The contralateral eye of each animal served as uninjected control. Five weeks post-injection, all animals were sacrificed. Both eyes, injected and contralateral uninjected, were dissected and ocular fluids were collected in 40 μl of PBS (containing protease inhibitors) in screw capped tubes and snap frozen until protein analysis. Posterior eyecups were placed into a fresh tube containing 50 μl of RNAlater solution and were broken down further with dissecting scissors. The dissected eyecups were then snap frozen in liquid nitrogen for 30 sec, and 50 uL RLT buffer containing p-mercaptoethanol was added before subjecting to further homogenisation using a homogeniser for 2 min. 200 uL RLT buffer with p-mercaptoethanol was added to the eyecup homogenates and mixed by pipetting up and down. All eyecup homogenates were stored for at least 24 hr at −80° C. before RNA extraction.

RNA Extraction and Quantitative RT-PCR

RNA was isolated from rat posterior eyecup tissues using RNeasy Mini kit as per manufacturer's protocol (Qiagen). Frozen eyecup homogenates were thawed on ice subjected to mechanical shredding by using a QIAshredder. Following shredding, 70% EtOH was added to the homogenate and the mixture was then transferred to a RNeasy column. The column was centrifuged and washed with RW1 buffer. The column membrane was then treated with DNaseI by incubation at room temperature for 15 mins before further washing with RW1 and RPE buffers. RNA was then eluted with RNase-free water in a final volume of 50 μL. RNA concentration of each sample was then measured using NeoDot nanospectrometer (Generon).

The reverse transcription was performed using the SuperScript III Reverse Transcriptase Kit (Invitrogen). Transgene-derived mRNA was quantified by using the following primers targeting the bGHpA sequence for murine samples: bGHpA FR 5′-CATCGCATTGTCTGAGTAGGT-3′ and bGHpA Rv 5′-AGCATGCCTGCTATTGTCTT-3′. All qRT-PCR was conducted using an CFX96 Touch Real-Time PCR Detection System (BioRad) with SYBR Green chemistry. As a standard, linearised transgene plasmid diluted to known concentrations was used. The SYBR Green applications were performed as follows: initial denaturation at 95° C. for 3 min followed by 40 cycles of 10 sec at 95° C. and 30 sec at 56° C.

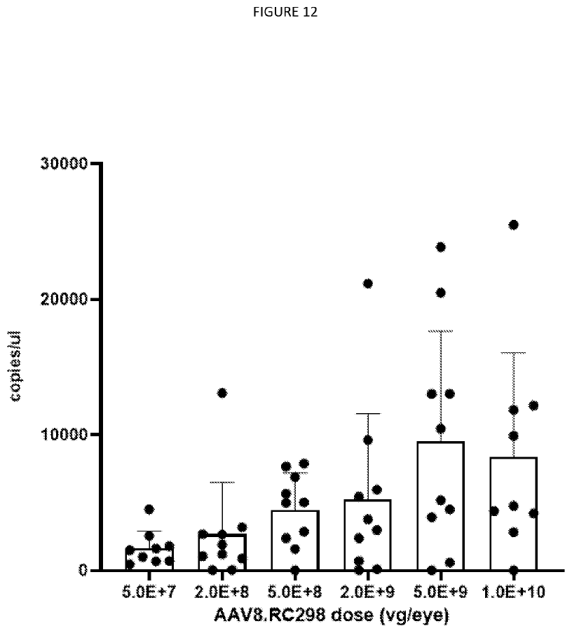

FIG. 12 shows the Aflibercept mRNA expression for Groups 1 to 6.

Quantitation of Aflibercept Expression

Unbound aflibercept in ocular fluids was measured using a quantitative sandwich type ELISA as per manufacturer's protocol (ImmunoGuide). In brief, diluted Aflibercept standards and ocular fluid samples were incubated in the microtiter plate coated with recombinant human vascular endothelial growth factor-A (rhVEGF-A). After incubation, the wells were washed and horseradish peroxidase (HRP) conjugated anti-human IgG monoclonal antibody was added to bind to the Fc portion of Aflibercept pre-captured by the rhVEGF-A on the surface of the wells. Following incubation, the wells were washed, and the bound enzymatic activity was detected by the addition of chromogen-substrate. The colour developed was proportional to the amount of Aflibercept in the sample or standard, and the optical density (OD) was measured with a photometer at 450 nm (reference at OD620 nm was optional).

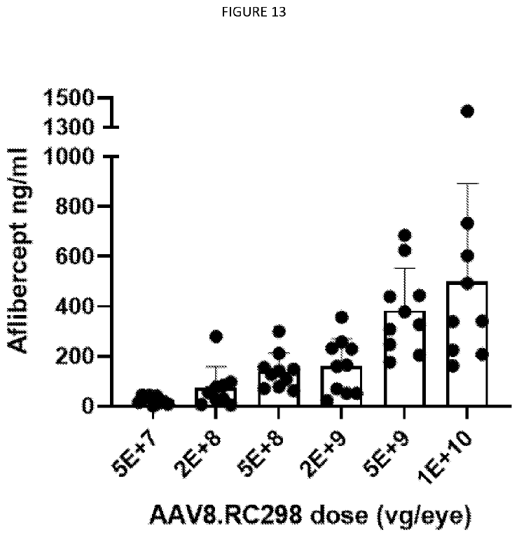

FIG. 13 shows the Aflibercept protein expression for Groups 1 to 6.

Bicistronic Expression Data

Bicistronic AAV vectors co-expressing antiVEGF (Aflibercept) and complement regulators (hCFI or FHL-1) were administered subretinally into the right eye (OD) of 10-week-old C57BL/6 male mice. Successful subretinal administrations were verified using spectral-domain optical coherence tomography (SD-OCT). The contralateral eye of each animal served as an uninjected control. After four weeks the mice were sacrificed, and ocular fluids and posterior eye cups were collected.

The study consisted of eight treatment groups:
Group 1: AAV8-RC313, 5×10⁷ vg/eye (n=10 mice);
Group 2: AAV8-RC313, 5×10⁸ vg/eye (n=10);
Group 3: AAV8-RC289, 5×10⁷ vg/eye (n=10);
Group 4: AAV8-RC289, 5×10⁸ vg/eye (n=10);
Group 5: AAV8-RC304, 5×10⁷ vg/eye (n=9);
Group 6: AAV8-RC304, 5×10⁸ vg/eye (n=10);
Group 7: AAV8-RC312, 5×10⁷ vg/eye (n=9);
Group 8: AAV8-RC312, 5×10⁸ vg/eye (n=10).

RNA Extraction and Quantitative RT-PCR

Posterior eyecups were placed into a fresh tube containing 50 μl of RNAlater solution and were broken down further with dissecting scissors. The dissected eyecups were then snap frozen in liquid nitrogen for 30 sec, and 50 uL RLT buffer containing p-mercaptoethanol was added before subjecting to further homogenisation using a homogeniser for 2 min. 200 uL RLT buffer with p-mercaptoethanol was added to the eyecup homogenates and mixed by pipetting up and down. All eyecup homogenates were stored for at least 24 hr at −80° C. before RNA extraction.

RNA was isolated from rat posterior eyecup tissues using RNeasy Mini kit as per manufacturer's protocol (Qiagen). Frozen eyecup homogenates were thawed on ice subjected to mechanical shredding by using a QIAshredder. Following shredding, 70% EtOH was added to the homogenate and the mixture was then transferred to a RNeasy column. The column was centrifuged and washed with RW1 buffer. The column membrane was then treated with DNaseI by incubation at room temperature for 15 mins before further washing with RW1 and RPE buffers. RNA was then eluted with RNase-free water in a final volume of 50 μL. RNA concentration of each sample was then measured using NeoDot nanospectrometer (Generon).

The reverse transcription was performed using the SuperScript III Reverse Transcriptase Kit (Invitrogen). Transgene-derived mRNA was quantified by using the following primers targeting the bGHpA sequence for murine samples: bGHpA FR 5′-CATCGCATTGTCTGAGTAGGT-3′ and bGHpA Rv 5′-AGCATGCCTGCTATTGTCTT-3′. All qRT-PCR was conducted using an CFX96 Touch Real-Time PCR Detection System (BioRad) with SYBR Green chemistry. As a standard, linearised transgene plasmid diluted to known concentrations was used. The SYBR Green applications were performed as follow: initial denaturation at 95° C. for 3 min followed by 40 cycles of 10 sec at 95° C. and 30 sec at 56° C.

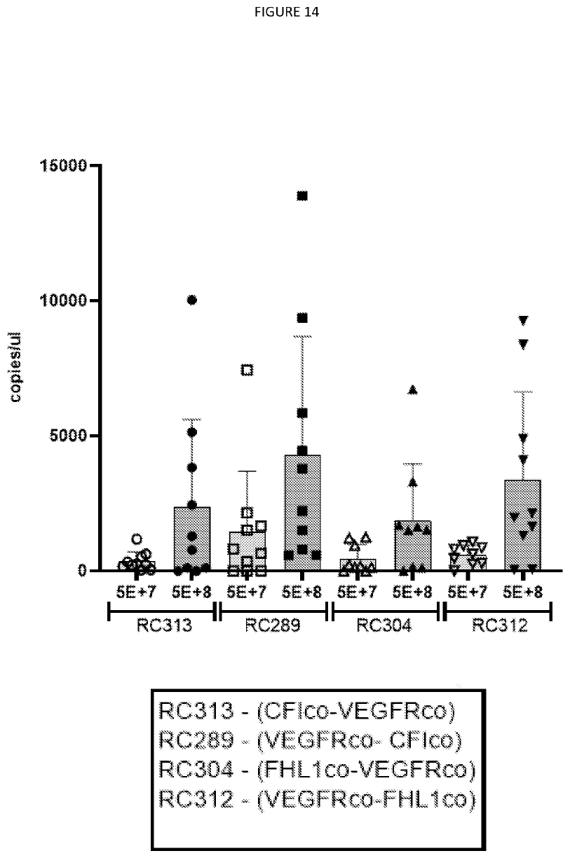

FIG. 14 shows the Aflibercept mRNA expression from the bicistronic vectors

Quantitation of Aflibercept and Complement Expression

Aflibercept

Unbound aflibercept in ocular fluids was measured using a quantitative sandwich type ELISA as per manufacturer's protocol (ImmunoGuide). In brief, diluted Aflibercept standards and ocular fluid samples were incubated in the microtiter plate coated with recombinant human vascular endothelial growth factor-A (rhVEGF-A). After incubation, the wells were washed and horseradish peroxidase (HRP) conjugated anti-human IgG monoclonal antibody was added to bind to the Fc portion of Aflibercept pre-captured by the rhVEGF-A on the surface of the wells. Following incubation, the wells were washed, and the bound enzymatic activity was detected by the addition of chromogen-substrate. The colour developed was proportional to the amount of Aflibercept in the sample or standard, and the optical density (OD) was measured with a photometer at 450 nm (reference at OD620 nm was optional).

CFI Protein Expression in Ocular Fluids

Human CFI in ocular fluids was quantified using MSD's electrochemiluminescence detection technology. An MSD Gold 96-well small spot streptavidin Sector plate was coated with biotinylated anti-CFI antibody shaking (850 RPM) for 1 hr at room temperature. CFI Standards (Complement Technology) and samples were diluted accordingly. The plate was washed with PBS-Tween 0.05% and 25 uL of standards, controls and samples were added to the plate. The plate was sealed and incubated shaking (850 RPM) at room temperature for 1 hour. The plate was then washed as previous and 25 uL of anti FI (anti-FI, A231) detection antibody was added to the wells and incubated shaking (850 RPM) at room temperature for 1 hour. Following incubation, the plate was washed as previous and 150 ul of 2×Read Buffer T was added to each well before reading on the MSD plate reader.

FHL-1 Protein Expression in Ocular Fluids

FHL-1 in ocular fluids was quantified using MSD's electrochemiluminescence detection technology. An MSD Gold 96-well streptavidin Sector plate was coated with biotinylated anti-FHL-1 antibody shaking (850 RPM) for 1 hr at room temperature. FHL-1 standards and samples were diluted accordingly. The plate was washed with PBS-Tween 0.05% and 25 uL of standards, controls and samples were added to the plate. The plate was sealed and incubated shaking (850 RPM) at room temperature for 1 hour. The plate was then washed as previous and 25 uL of anti FH detection antibody (anti-FH, MA170057, Invitrogen) was added to the wells and incubated shaking (850 RPM) at room temperature for 1 hour. Following incubation, the plate was washed as previous and 150 ul of 2×Read Buffer T was added to each well before reading on the MSD plate reader.

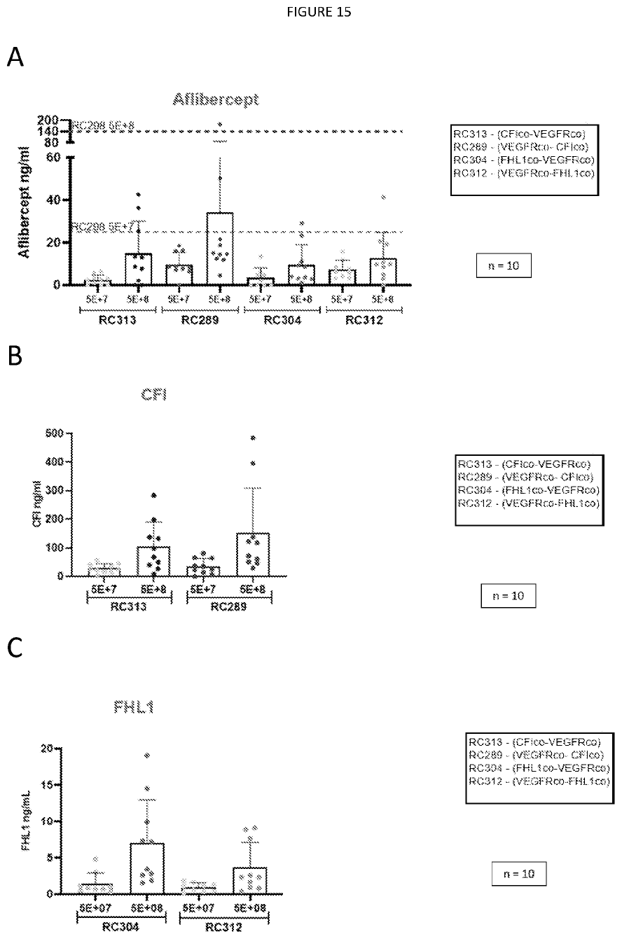

FIG. 15 shows the Aflibercept, CFI and FH L1 protein expression from the bicistronic vectors.

### Example 7—In Vivo Efficacy in Murine Laser-Induced Choroidal Neovascularisation Model

Five groups of mice received unilateral (right eye) subretinal injections of different AAV vectors. Successful subretinal treatment administration was confirmed immediately after AAV administration using spectral-domain optical coherence tomography (SD-OCT). Four weeks after administration of the vectors, CNV was induced by laser photocoagulation. In brief, anaesthetized animals received a drop of 0.5% tropicamide (Oftan Tropicamid, Santen Oy) to dilate the pupils. A drop of Viscotears (Dr. Gerhard Mann Chem. -Pharm., Germany) was applied on the eye and a coverslip was used to applanate the cornea. Three laser lesions were executed unilaterally on the right eye around the optic nerve head using a 532 nm diode laser (spot size: 100 μm; power: 130 mW; time: 120 ms. Oculight TX. Iridex Corp., USA). The successful production of CNV lesions was confirmed by using SD-OCT and fluorescein angiography (FA). Immediately after CNV induction, an additional group of mice received intravitreal administration of aflibercept. Mice were imaged again at days four and seven post lasering with SD-OCT and FA, and then sacrificed.

Lasered eyes were enucleated and dissected. Choroidal flat mounts were prepared and stained with fluorescein-labelled Griffonia Simplicifolia Lectin I (GSLI) isolectin B4 (FL-1201, Vector Laboratories, USA) to evaluate the neovascularization. Briefly, the flat mounts were washed with Tris-buffered saline (TBS) and blocked with 10% normal goat serum (NGS), 0.5% Triton X-100 in TBS pH 7.4 (TBST) for 1 hour in room temperature (RT). Samples were washed with TBS and incubated with fluorescein-labelled Isolectin GS-IB4 (1:200, Vector Laboratories) overnight at +4° C. in 1% NGS diluted in 0.1% TBST. Thereafter the samples were washed 3×10 min with 1% NGS diluted in 0.1% TBST, counterstained with DAPI and mounted with Fluoroshield™ mounting medium (Sigma-Aldrich) on microscopic slides. Choroidal samples were imaged using a DMi8 THUNDER 3D microscope (Leica Microsystems, Germany). The stained areas were outlined and the stained area was measured using the image processing software FIJI (Schindelin et al. 2012).
Group 1: RC268, null, 5 E8 on Day −28 and CNV induction;
Group 2: RC289, 5 E7 on Day −28 and CNV induction;
Group 3: RC289, 5 E8 on Day −28 and CNV induction;
Group 4: RC304, 5 E7 on Day −28 and CNV induction;
Group 5: RC304, 5 E8 on Day −28 and CNV induction;
Group 6: CNV induction and aflibercept (80 μg/eye) treatment on day 0.

Quantitation of CNV Leakage Area and CNV Lesion Size

Fluorescein Angiography (FA): Vascular leakage at the choroid level was examined using a Heidelberg Spectralis HRA system (Heidelberg Engineering, Germany). Briefly, a drop of 0.5% tropicamide (Oftan Tropicamid. Santen Oy) was administered on the cornea of the anaesthetized mouse, in order to dilate the pupils, and the mouse was placed onto the mouse holder. After aligning the optic nerve head at the retina level, with the use of the infrared reflectance camera, a solution of 2.5% sodium fluorescein (Sigma-Aldrich, Finland) was administered as a s.c. injection (30 μl/10 g). Consecutive fluorescent images (Sensitivity: 45; ART Mean: 5 frames) were taken every 60 sec from the retinal and choroidal focus levels for a period of 5 min after the fluorescein administration.

Spectral Domain Optical Coherence Tomography (SD-OCT): SD-OCT was performed to verify the subretinal administration, prior and after the CNV induction, and at days 4 and 7 after CNV induction. Immediately after the FA imaging the mouse was examined using the SD-OCT system Envisu R2200 (Bioptigen Inc./Leica Microsystems, USA). The scanned area covers a 1.4×1.4 mm² of the retina centered around the optic nerve. Each scan is composed of 100 B Scans each one composed of 1000 A Scans.

CNV leakage area as assessed by FA fundus imaging and subsequent AI image analysis. FA scans were analyzed by a proprietary algorithm, which uses a combination of convolutional neural network (CNN) designed for semantic segmentation and traditional computer vision algorithms. The neural network was trained to recognize and quantify CNV lesions using transfer learning approach. The results from the model were reviewed and adjusted if necessary by a scientist blinded to the treatments

The lasered areas were qualitatively graded as being leaky or non-leaky from FA scans and verified from SD-OCT scans.

Data Analysis

Quantitative data was graphed, analyzed and presented as mean±standard deviation (SD). Statistical analyses were performed using the GraphPad Prism software (v8.0.1 GraphPad Software. USA). Differences were considered statistically significant at the p<0.05 level.

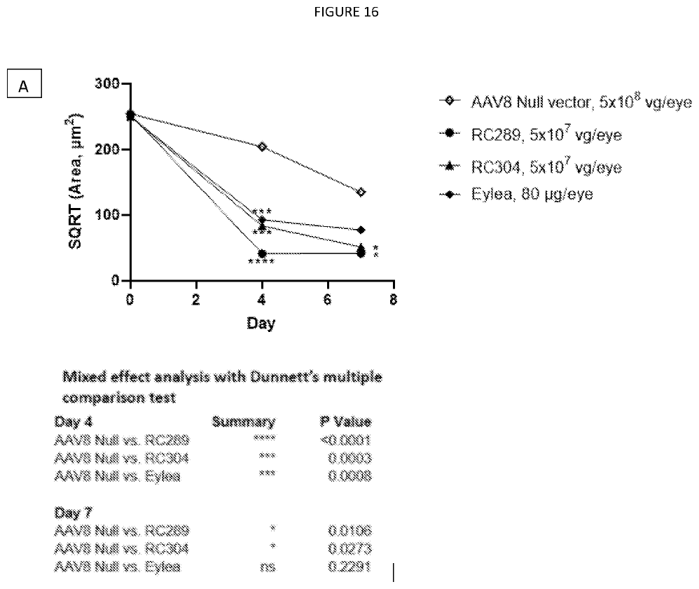

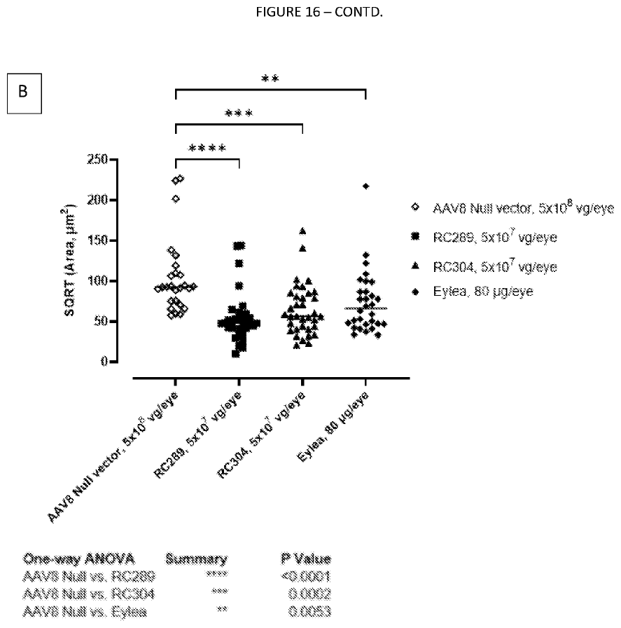

The results are shown in FIG. 16.

All publications mentioned in the above specification are herein incorporated by reference. Various modifications and variations of the disclosed agents, compositions, uses and methods of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

1. A polynucleotide comprising nucleotide sequences encoding (i) an anti-VEGF entity and (ii) a negative complement regulator.

2. The polynucleotide of claim 1, wherein the anti-VEGF entity is selected from an Ig fusion protein, an antibody, a polypeptide, a peptide, a non-antibody scaffold, an antisense oligonucleotide, siRNA, shRNA, CRISPR-guide strand and an aptamer.

3. The polynucleotide of claim 2, wherein the anti-VEGF entity is selected from aflibercept, ranibizumab, bevacizumab, brolucizumab and pegaptanib.

4. The polynucleotide of claim 3, wherein the anti-VEGF entity is aflibercept.

5. The polynucleotide of claim 1, wherein the negative complement regulator is selected from Complement Factor I (CFI), Complement Factor H Like Protein 1 (FHL1), Complement Factor H (CFH), Complement receptor type 1 (CR1), Membrane Cofactor Protein (MCP), Complement decay-accelerating factor (DAF), MAC-inhibitory protein (MAC-IP), C1-inhibitor, anaphylatoxins inhibitor, C4b binding protein (C4BP), clusterin, vitronectin, and variants and fragments thereof.

6. The polynucleotide of claim 5, wherein the negative complement regulator is selected from CFI and FHL1 and variants and fragments thereof.

7. The polynucleotide of claim 4, wherein the nucleotide sequence encoding aflibercept has at least 75% sequence identity to SEQ ID NO: 11.

8. The polynucleotide am of claim 6, wherein the nucleotide sequence encoding CFI has at least 75% sequence identity to SEQ ID NO: 35 or 36 or the nucleotide sequence encoding FHL-1 has at least 75% sequence identity to SEQ ID NO: 41.

9. The polynucleotide of claim 3, wherein the polynucleotide comprises the nucleotide sequence of any one of SEQ ID NO: 45 to 54, or a nucleotide sequence that has at least 75% sequence identity thereto.

10. The polynucleotide of claim 1, wherein the polynucleotide is less than or equal to 4.7 kb.

11. A vector comprising the polynucleotide of claim 1.

12. The vector of claim 11, wherein the vector is an adeno-associated viral (AAV) vector.

13. A pharmaceutical composition comprising the polynucleotide of claim 1 in combination with a pharmaceutically acceptable carrier, diluent or excipient.

14. (canceled)

15. A method of treating or preventing a complement-mediated disorder of the eye, comprising administering the polynucleotide of claim 1 to a subject in need thereof.

16. The method of claim 15, wherein the complement-mediated disorder of the eye is wet AMD or dry AMD.

17. The method of claim 15, wherein i) the complement-mediated disorder of the eye is wet AMD and the administration prevents and/or treats onset of dry AMD in the subject, or ii) the complement-mediated disorder of the eye is dry AMD and the administration prevents and/or treats onset of wet AMD in the subject, or iii) the complement-mediated disorder of the eye is AMD and the administration prevents and/or treats dry AMD and wet AMD simultaneously.

18. The method of claim 17, wherein the subject has wet AMD and the administration prevents and/or treats onset of dry AMD in the subject.

19. The method of claim 15, wherein:
(i) the formation of geographic atrophy is prevented or reduced, and/or the amount of geographic atrophy is reduced or wherein the progression of geographic atrophy is slowed;
(ii) there is at least a 10% reduction in the increase in geographic atrophy area over the 12 months following administration to a treated eye of the subject, relative to an untreated eye over the same period; or
(iii) administration of the polynucleotide
increases the level of C3b-inactivating and iC3b-degradation activity in the subject, or in an eye of the subject.

20. The method of claim 15, wherein i) neovascularization is prevented or reduced, ii) vascular leakage is prevented or reduced and/or iii) retina edema is prevented or reduced.

21. The method of claim 15, wherein the polynucleotide is administered intraocularly.

22. A method of treating and/or preventing wet AMD and/or dry AMD in a subject in need thereof, comprising administering to the subject (i) an anti-VEGF entity; and (ii) a negative complement regulator, or nucleotide sequences encoding therefor simultaneously, separately, or sequentially.

23. The method of claim 22, wherein the subject has wet AMD and the administration prevents and/or treats onset of dry AMD in the subject.

24. The method of claim 22, wherein the anti-VEGF entity is selected from an Ig fusion protein, an antibody, a polypeptide, a peptide, a non-antibody scaffold, an antisense oligonucleotide, siRNA, shRNA, CRISPR-guide strand and an aptamer, preferably wherein the anti-VEGF entity is selected from aflibercept, ranibizumab, bevacizumab, brolucizumab and pegaptanib.

25. The method of claim 22, wherein the negative complement regulator is selected from Complement Factor I (CFI), Complement Factor H Like Protein 1 (FHL1), Complement Factor H (CFH), Complement receptor type 1 (CR1), Membrane Cofactor Protein (MCP), Complement decay-accelerating factor (DAF), MAC-inhibitory protein (MAC-IP), C1-inhibitor, anaphylatoxins inhibitor, C4b binding protein (C4BP), clusterin, vitronectin and variants and fragments thereof.

26. A product comprising (i) an anti-VEGF entity; and (ii) Factor I, or nucleotide sequences encoding therefor, as a combined preparation.

27. A method of treating or preventing a complement-mediated ocular disorder comprising administering the product of claim 26 to a subject in need thereof, wherein the anti-VEGF entity and Factor I, or nucleotide sequences encoding therefor are administered simultaneously, separately, or sequentially.

28. The method of claim 22, wherein:
(i) the formation of geographic atrophy is prevented or reduced, and/or the amount of geographic atrophy is reduced or wherein the progression of geographic atrophy is slowed;
(ii) there is at least a 10% reduction in the increase in geographic atrophy area over the 12 months following administration to a treated eye of the subject, relative to an untreated eye over the same period; or
(iii) administration of the isolated polynucleotide, vector or pharmaceutical composition increases the level of C3b-inactivating and iC3b-degradation activity in the subject, or in an eye, of the subject.

29. The method of claim 22, wherein i) neovascularization is prevented or reduced, ii) vascular leakage is prevented or reduced and/or iii) retina edema is prevented or reduced.
